(19) United States
(12) Patent Application Publication
(10) Pub. No.: US 2023/0159556 A1
(43) Pub. Date: May 25, 2023

(54) NOVEL PROTEIN KINASE INHIBITORS

(71) Applicant: TENOVA PHARMACEUTICALS INC., San Diego, CA (US)

(72) Inventors: Jianhua Guo, San Diego, CA (US); Bo Cheng, San Diego, CA (US); Hongyi Chen, San Diego, CA (US); Junhua Fan, San Diego, CA (US)

(21) Appl. No.: 17/917,119

(22) PCT Filed: Apr. 19, 2021
(86) PCT No.: PCT/US2021/027944

§ 371 Date: Oct. 5, 2022

Related U.S. Application Data
(60) Provisional application No. 63/012,274, filed on Apr. 20, 2020.

(51) Int. Cl. C07D 498/08; A61P 35/00; C07F 9/6561; C07D 487/08

(52) U.S. Cl. CPC C07D 498/08; A61P 35/00; C07F 9/6561; C07D 487/08

(57) ABSTRACT

The present disclosure describes novel protein kinase inhibitors and methods for preparing them. The pharmaceutical compositions comprising such protein kinase inhibitors and methods of using them for treating cancer and other diseases, conditions, or disorders, which respond to the inhibition of epidermal growth factor receptor (EGFR), anaplastic lymphoma kinase (ALK) activity, or a combination thereof, are also described.

### CROSS-REFERENCE TO RELATED APPLICATIONS

This application claims the benefit of U.S. Provisional Application No. 63/012,274, filed Apr. 20, 2020; which is incorporated by reference by its entirety.

### BACKGROUND

Protein kinases regulate various cellular activities, including proliferation, survival, apoptosis, metabolism, transcription, differentiation, and a wide array of other cellular processes. Kinase Inhibitors have been found to be useful for the treatment of numerous diseases such as cancers, inflammatory diseases, central nervous system (CNS) disorders, cardiovascular diseases, and complications of diabetes.

The epidermal growth factor receptor (EGFR) family of receptor tyrosine kinases regulate cell proliferation, survival, adhesion, migration and differentiation. Deregulated kinase activity of epidermal growth factor receptor (EGFR) is responsible for the pathogenesis of non-small cell lung cancer (NSCLC). Deletion of Glu746-Ala750 (d746-750) in exon 19 and the L858R point mutation in exon 21 are the most prevalent EGFR mutations. The first-generation EGFR inhibitors (gefitinib and erlotinib) targeting such oncogenic mutants have proved to be successful. However, a secondary somatic mutation at the gatekeeper position (T790M) was discovered to cause drug resistance in NSCLC patients after treating with the first-generation EGFR inhibitors. Second-generation EGFR inhibitors such as afatinib, dacomitinib, and meratinib are quite effective against the acquired drug resistance. Third-generation EGFR inhibitors such as osimertinib exhibit characteristic specificity toward the drug-resistant L8585R/T790M and d746-750/T790M mutants and thus avoid a variety of severe side effects owing to the simultaneous inhibition of wild-type EGFR for the second-generation EGFR inhibitors. However, the emergence of a tertiary mutation (C797S) in EGFR has provoked new drug resistance. Fourth-generation EGFR inhibitors of the triple mutants involving C797S have been actively pursued to overcome the resistance.

Anaplastic lymphoma kinase (ALK) is a member of the insulin receptor tyrosine kinase family. Chromosomal rearrangements of anaplastic lymphoma kinase (ALK) are detected in 3% to 7% of non-small cell lung cancers (NSCLC). A few ALK inhibitors have been successfully approved or in clinical study for treatment of EML4-ALK rearrangement. First-generation ALK inhibitor crizotinib demonstrated clear clinical benefits to treat ALK-positive NSCLC patients. However, a majority of patients developed resistance to crizotinib treatment successively. Several more potent second- and third-generation inhibitors have been identified to combat disease resistance, such as ceritinib, alectinib, brigatinib and lorlatinib.

Despite advancements in the art, there remains a need for better cancer treatments and better anticancer compounds.

### SUMMARY

The present disclosure relates to certain optionally substituted macrocyclic compounds comprising at least three aromatic rings within the macrocyclic ring system, such as N-(3⁵-bromo-1⁶-methoxy-1⁴-(4-(4-methylpiperazin-1-yl)piperidin-1-yl)-6-oxa-2,4-diaza-3(2,4)-pyrimidina-1(1,3),5(1,4)-dibenzenacycloundecaphane-5²-yl)-N-methylmethanesulfonamide, or a pharmaceutical composition thereof. Such a macrocyclic compound comprises novel pyrimidine, pyridine, or triazine derivatives, or a combination thereof, such as any one of the compounds represented by Formula 1, or any one of other novel compounds described herein, or a pharmaceutically acceptable salt thereof (referred to collectively herein as a “subject compound”). For example, some embodiments include a subject compound that is: 3⁵-chloro-1⁶-methoxy-1⁴-(4-methylpiperazin-1-yl)-5²-(methylsulfonyl)-6-oxa-2,4-diaza-3(2,4)-pyrimidina-1(1,3),5(1,4)-dibenzenacyclononaphane, optionally substituted N-(3⁵-chloro-1⁶-methoxy-1⁴-(4-methylpiperazin-1-yl)-6-oxa-2,4-diaza-3(2,4)-pyrimidina-1(1,3),5(1,4)-dibenzenacyclononaphane-5²-yl)-N-methylmethanesulfonamide, optionally substituted N-(3⁵-chloro-1⁶-methoxy-1⁴-(4-methylpiperazin-1-yl)-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-chloro-1⁶-methoxy-1⁴-(4-methylpiperazin-1-yl)-6-oxa-2,4-diaza-3(2,4)-pyrimidina-1(1,3),5(1,4)-dibenzenacyclodecaphane-5²-yl)-N-methylmethanesulfonamide, optionally substituted N-(3⁵-chloro-1⁴-((2-(dimethylamino)ethyl)(methyl)amino)-1⁶-methoxy-6-oxa-2,4-diaza-3(2,4)-pyrimidina-1(1,3),5(1,4)-dibenzenacycloundecaphane-5²-yl)-N-methylmethanesulfonamide, optionally substituted 3⁵-chloro-1⁶-methoxy-1⁴-(4-methylpiperazin-1-yl)-5²-(methylsulfonyl)-6-oxa-2,4-diaza-3(2,4)-pyrimidina-1(1,3),5(1,4)-dibenzenacyclodecaphane, optionally substituted N-(3⁵-chloro-1⁴-((2-(dimethylamino)ethyl)(methyl)amino)-1⁶-methoxy-6-oxa-2,4-diaza-3(2,4)-pyrimidina-1(1,3),5(1,4)-dibenzenacyclononaphane-5²-yl)-N-methylmethanesulfonamide, optionally substituted N-(3⁵-chloro-1⁶-methoxy-1⁴-(4-methyl piperazin-1-yl)-6-oxa-2,4-diaza-3(2,4)-pyrimidina-1(1,3),5(1,4)-dibenzenacycloundecaphane-5²-yl)-N-methylethanesulfonamide, optionally substituted N-(3⁵-chloro-1⁶-methoxy-1⁴-(4-(4-methylpiperazin-1-yl)piperidin-1-yl)-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-chloro-1⁶-methoxy-1⁴-(4-methylpiperazin-1-yl)-6-oxa-2,4-diaza-3(2,4)-pyrimidina-1(1,3),5(1,4)-dibenzenacycloundecaphane-5²-yl)-N-ethylmethanesulfonamide, optionally substituted N-(3⁵-fluoro-1⁶-methoxy-1⁴-(4-methylpiperazin-1-yl)-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-bromo-1⁶-methoxy-1⁴-(4-methylpiperazin-1-yl)-6-oxa-2,4-diaza-3(2,4)-pyrimidina-1(1,3),5(1,4)-dibenzenacycloundecaphane-5²-yl)-N-methylmethanesulfonamide, optionally substituted N-(1⁶-methoxy-1⁴-(4-methyl piperazin-1-yl)-3⁵-(trifluoromethyl)-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-chloro-1⁶-methoxy-1⁴-(4-methylpiperazin-1-yl)-6-oxa-2,4-diaza-3(2,4)-pyrimidina-1(1,3),5(1,4)-dibenzenacyclododecaphane-5²-yl)-N-methylmethanesulfonamide, optionally substituted N-(3⁵-chloro-1⁶-methoxy-14-(4-(4-methylpiperazin-1-yl)piperidin-1-yl)-6-oxa-2,4-diaza-3(2,4)-pyrimidina-1(1,3),5(1,4)-dibenzenacyclododecaphane-5²-yl)-N-methylmethanesulfonamide, optionally substituted N-(3⁵-chloro-1⁶-methoxy-1⁴-(4-methylpiperazin-1-yl)-6-oxa-2,4-diaza-3(2,4)-pyrimidina-1(1,3),5(1,4)-dibenzenacycloundecaphane-5²-yl)methanesulfonamide, optionally substituted N-(3⁵-chloro-1⁶-methoxy-1⁴-(4-methylpiperazin-1-yl)-6-oxa-2,4-diaza-3(2,4)-pyrimidina-1(1,3),5(1,4)-dibenzenacycloundecaphane-5²-yl)-N-methylpropane-2-sulfonamide, optionally substituted (3⁵-chloro-1⁶-methoxy-1⁴-(4-methylpiperazin-1-yl)-6-oxa-2,4-diaza-3(2,4)-pyrimidina-1(1,3),5(1,4)-dibenzenacycloundecaphane-5²-yl)dimethylphosphine oxide, optionally substituted 3⁵-chloro-1⁶-methoxy-1⁴-(4-(4-methylpiperazin-1-yl)piperidin-1-yl)-5²-(methylsulfonyl)-6-oxa-2,4-diaza-3(2,4)-pyrimidina-1(1,3),5(1,4)-dibenzenacyclododecaphane, optionally substituted N-(3⁵-chloro-1⁶-methoxy-1⁴-(4-(1-methylpiperidin-4-yl)piperazin-1-yl)-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-chloro-1⁶-ethoxy-1⁴-(4-methylpiperazin-1-yl)-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-chloro-1⁶-isopropoxy-1⁴-(4-methyl piperazin-1-yl)-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-chloro-1⁶-methoxy-1⁴-(4-methylpiperazin-1-yl)-6-oxa-2,4-diaza-3(2,4)-pyrimidina-1(1,3),5(1,4)-dibenzenacyclododecaphane-5²-yl)-N-ethylmethanesulfonamide, optionally substituted N-(3⁵-chloro-1⁴-(4-(dimethylamino)piperidin-1-yl)-1⁶-methoxy-6-oxa-2,4-diaza-3(2,4)-pyrimidina-1(1,3),5(1,4)-dibenzenacycloundecaphane-5²-yl)-N-methylmethanesulfonamide, optionally substituted 3⁵-chloro-1⁶-methoxy-N,N-dimethyl-1⁴-(4-methylpiperazin-1-yl)-6-oxa-2,4-diaza-3(2,4)-pyrimidina-1(1,3),5(1,4)-dibenzenacyclododecaphane-5²-carboxamide, optionally substituted 3⁵-chloro-5²-(isopropylsulfonyl)-16-methoxy-1⁴-(4-(4-methylpiperazin-1-yl)piperidin-1-yl)-6-oxa-2,4-diaza-3(2,4)-pyrimidina-1(1,3),5(1,4)-dibenzenacycloundecaphane, optionally substituted N-(3⁵-chloro-1⁴-(3-(dimethylamino)pyrrolidin-1-yl)-1⁶-methoxy-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-chloro-1⁶-methoxy-1⁴-((cis-3,6)-5-methylhexahydropyrrolo[3,4-c]pyrrol-2(1H)-yl)-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-bromo-1⁶-methoxy-1⁴-(4-(4-methylpiperazin-1-yl)piperidin-1-yl)-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-bromo-1⁶-methoxy-1⁴-(4-(4-methylpiperazin-1-yl)piperidin-1-yl)-6-oxa-2,4-diaza-3(2,4)-pyrimidina-1(1,3),5(1,4)-dibenzenacycloundecaphane-5²-yl)-N-ethylmethanesulfonamide, optionally substituted N-(1⁴-(4-acetylpiperazin-1-yl)-3⁵-chloro-1⁶-methoxy-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-chloro-1⁶-methoxy-1⁴-(4-morpholinopiperidin-1-yl)-6-oxa-2,4-diaza-3(2,4)-pyrimidina-1(1,3),5(1,4)-dibenzenacycloundecaphane-5²-yl)-N-methylmethanesulfonamide, optionally substituted (3⁵-bromo-1⁶-methoxy-1⁴-(4-(4-methylpiperazin-1-yl)piperidin-1-yl)-6-oxa-2,4-diaza-3(2,4)-pyrimidina-1(1,3),5(1,4)-dibenzenacycloundecaphane-5²-yl)dimethylphosphine oxide, optionally substituted (3⁵-bromo-1⁶-methoxy-1⁴-(4-(4-methylpiperazin-1-yl)piperidin-1-yl)-6-oxa-2,4-diaza-3(2,4)-pyrimidina-1(1,3),5(1,4)-dibenzenacyclododecap ha ne-5²-yl)dimethylphosphine oxide, optionally substituted (3⁵-bromo-1⁶-methoxy-1⁴-(4-methylpiperazin-1-yl)-6-oxa-2,4-diaza-3(2,4)-pyrimidina-1(1,3),5(1,4)-dibenzenacyclodecaphane-5²-yl)dimethylphosphine oxide, optionally substituted N-(3⁵-bromo-1⁴-(4-((2-(dimethylamino)ethyl)(methyl)amino)piperidin-1-yl)-1⁶-methoxy-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-bromo-1⁴-(4-(dimethylamino)-[1,4′-bipiperidin]-1′-yl)-1⁶-methoxy-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-bromo-1⁶-methoxy-1⁴-(2-methyl-2,7-diazaspiro[3.5]nonan-7-yl)-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-chloro-1⁶-methoxy-1⁴-(4-(4-methylpiperazin-1-yl)piperidin-1-yl)-10-oxo-6-oxa-2,4,11-triaza-3(2,4)-pyrimidina-1(1,3),5(1,4)-dibenzenacycloundecaphane-5²-yl)-N-methylmethanesulfonamide, optionally substituted N-(3⁵-chloro-1⁶-methoxy-1⁴-(4-(4-methylpiperazin-1-yl)piperidin-1-yl)-11-oxo-6-oxa-2,4,12-triaza-3(2,4)-pyrimidina-1(1,3),5(1,4)-dibenzenacyclododecaphane-5²-yl)-N-methylmethanesulfonamide, or optionally substituted N-(3⁵-chloro-1⁶-methoxy-1⁴-(4-(4-methylpiperazin-1-yl)piperidin-1-yl)-8-oxo-2,4,9-triaza-3(2,4)-pyrimidina-1(1,3),5(1,4)-dibenzenacyclododecaphane-5²-yl)-N-methylmethanesulfonamide, or optionally substituted N-(3⁵-chloro-1⁶-methoxy-1⁴-(4-(4-methylpiperazin-1-yl)piperidin-1-yl)-10-oxo-2,4,9-triaza-3(2,4)-pyrimidina-1(1,3),5(1,4)-dibenzenacyclododecaphane-5²-yl)-N-methylmethanesulfonamide.

This disclosure also relates to methods for using these subject compounds described herein. The methods disclosed herein include the use of the subject compounds to treat, ameliorate or prevent a condition which responds to the inhibition of epidermal growth factor receptor (EGFR), anaplastic lymphoma kinase (ALK) activity, or a combination thereof.

Some embodiments include a compound represented by Formula 1:

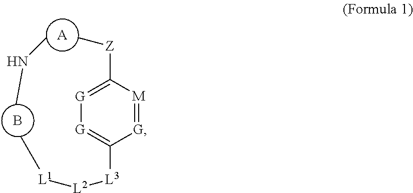

or a pharmaceutically acceptable salt thereof; wherein

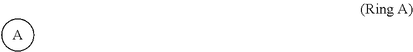

is an optionally substituted 6-membered aromatic heterocyclic ring or an optionally substituted 9-membered fused aromatic bicyclic heterocyclic ring;

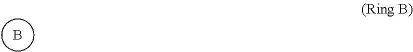

is an optionally substituted 5- or 6-membered aromatic ring, or an optionally substituted 10- or 13-membered fused bicyclic ring containing one 5- or 6-membered aromatic ring and one 5, 6, or 7-membered saturated ring; Z is O, S(O)₀₋₂, CR^{A1} R^{B1}, or NR^{A}; M is independently CR¹ or N; each G is independently CR or N; L¹ and L³ are independently a covalent bond, O, NR^{A}, S(O)₀₋₂, CR^{A1}R^{B1}, CR^{A1}═CR^{B1}, —C(O)NR^{A}—, —NR^{A}(CO)—, S(O)₁₋₂NR^{A}, or NR^{A}C(O)NR^{B}; L² is an optionally substituted C₁₋₁₂ alkylene, Cₘ alkylene-C(O)NR^{A}—Cₙ alkylene, Cₘ alkylene-NR^{A}(CO)—C, alkylene, or Cₘ alkylene-O—C, alkylene, wherein m is 1 to 12, n is 1 to 12, provided that the sum of m and n is no more than 12, wherein L² has, as chemically appropriate, 0, 1, 2, 3, 4, 5, 6, 7, 8, 9, 10, 11, or 12 substituents, and the substituents of L² are independently F, Cl, Br, I, OH, ═O, C₁₋₆ alkyl, or C₁₋₆ cycloalkyl; each R^{A1} and each R^{B1} are independently H, F, Cl, Br, I, or C₁₋₆ hydrocarbyl; each R is independently H, F, Cl, Br, I, —NR^{A}R^{B}, C₁₋₆ hydrocarbyl, —OH, —CN, —NO₂, —O—C₁₋₆ alkyl, or —C(O)O—C₁₋₆ alkyl; R¹ is H, F, Cl, Br, I, —NR^{A}R^{B}, C₁₋₆ hydrocarbyl, —OH, —CN, —NO₂, —O—C₁₋₆ alkyl, —C(O)O—C₁₋₆ alkyl, —S(O)₁₋₂R^{A}; —P(O)R^{A}R^{B}, —NR^{A}S(O)₂R^{B}, S(O)₂NR^{A}R^{B}, C(O)NR^{A}R^{B}, —NR^{A}C(O)R^{A}R^{B}; each R^{A} and each R^{B} are independently H or C₁₋₆ hydrocarbyl; and wherein each R^{A}, each R^{B}, each R^{A1}, each R^{B1}, each R, and each R¹ are independently optionally halogenated.

Some embodiments include a compound of Formula 1, wherein Ring A is:

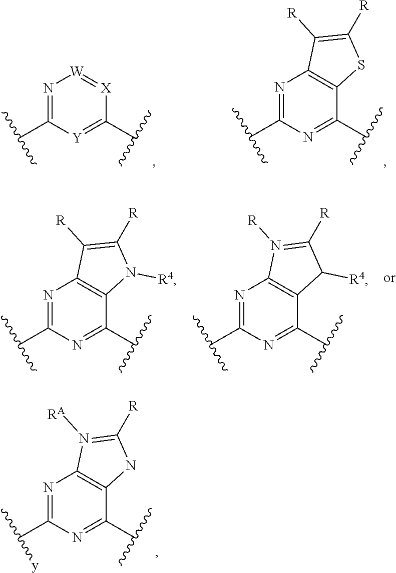

wherein each right side of the above structures is directly attached to Z in the Formula of claim 1; W is N or CR²; X is N or CR³; Y is N

### or CR⁴; and Ring B is:

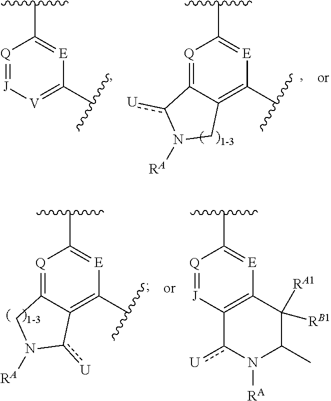

wherein L¹-L² is an optionally substituted C₁₋₈ alkylene and L³ is O; wherein each top side of the above structures is linked to Ring A via NH in the Formula of claim 1; each of the above structures of Ring A and Ring B is optionally substituted; each E is independently CR, NR^{A}, O, or S; each Q is independently CR⁵, NR^{A}, O, or S; each J is independently a bond, CR⁶, or N; V is CR², NR^{A}, O, or S; R² and R³ are independently H, F, Cl, Br, I, —NR^{A}R^{B}, C₁₋₆ hydrocarbyl, —OH, —CN, —NO₂, —O—C₁₋₆ alkyl, —C(O)O—C₁₋₆ alkyl, or —NR^{A}C(O)O—C₁₋₆ alkyl; R⁴, R⁵, and R⁶ are independently H, F, Cl, Br, I, —NR^{A}R^{B}, C₁₋₆ hydrocarbyl, —OH, —CN, —NO₂, O—C₁₋₆ alkyl, or —C(O)O—C₁₋₆ alkyl; R² and R³ may connect and together with the ring containing A to form a fused ring; R⁵ and R⁶ may connect and together with the ring containing A to form a fused ring; R⁷ is H, F, Cl, Br, I, NR^{A}R^{B}, —NR^{A} (CR^{A1}R^{B1})₁₋₃—NR^{A}R^{B}, C₁₋₆ hydrocarbyl, —OH, —CN, —NO₂, O—C₁₋₆ alkyl, or —C(O)O—C₁₋₆ alkyl, —NR^{A}S(O)₂R^{B}, —S(O)₂NR^{A}R^{B}, —C(O)NR^{A}R^{B}, —NR^{A}C(O)R^{A}R^{B}, —NR^{A}C(O)NR^{A}R^{B}, OC(O)NR^{A}R^{B}, CR^{A1}R^{B1}C(O)NR^{A}R^{B}, an optionally substituted 5- or 6-membered saturated mono-cyclic ring containing 1 or 2 ring N atoms and 0 to 1 ring O atom, or an optionally substituted 8 to 12 membered saturated bicyclic ring system containing 2 to 3 ring N atoms and 0 to 1 ring O atom; and wherein each R², each R³, each R⁴, each R⁵, each R⁶, and each R⁷ are independently optionally halogenated.

Some embodiments include a compound of Formula 1, wherein Ring A is:

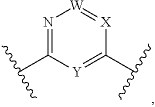

and wherein the Ring A-Z is:

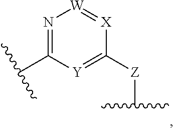

Some embodiments include a compound of Formula 1, wherein Ring A is:

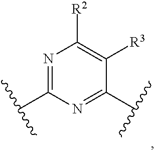

Some embodiments include a compound of Formula 1, which is further represented by Formula 1a, 1b, 1c, 1d, 1e, 1f, 1g, or 1h:

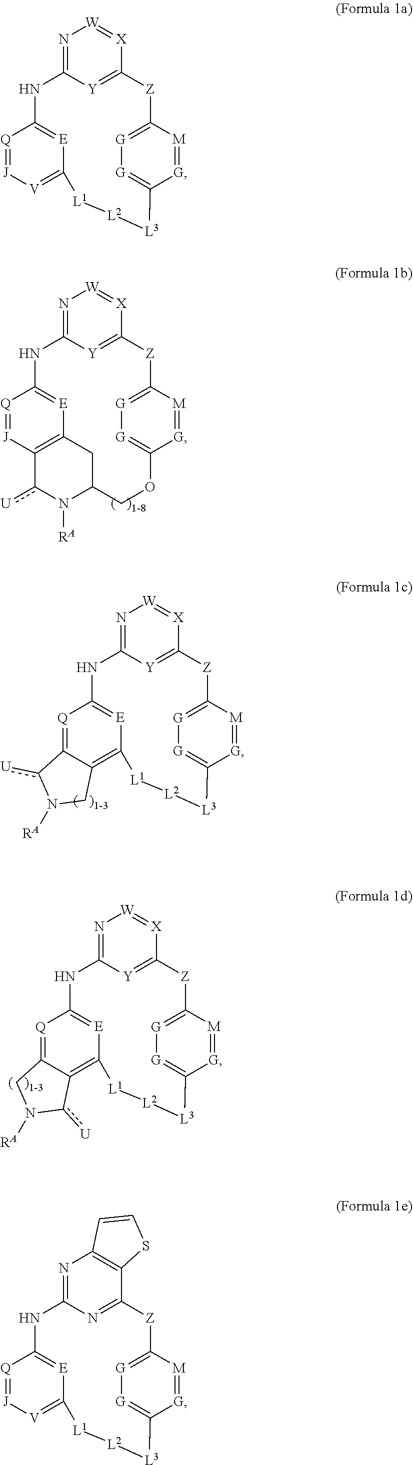

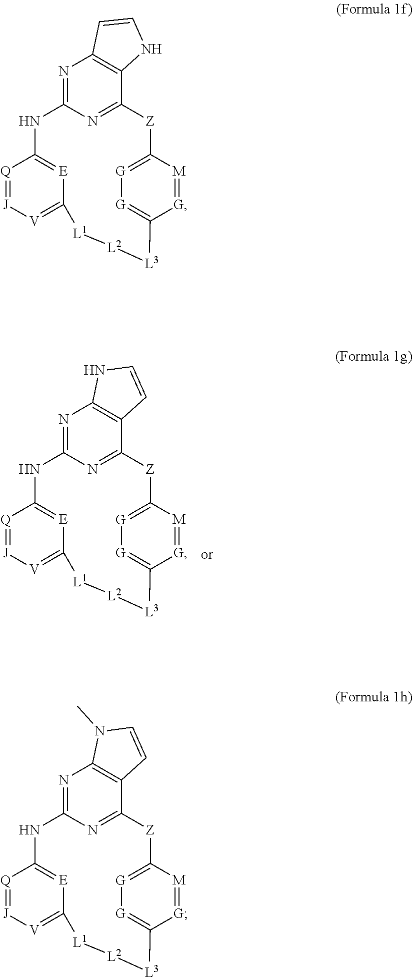

or a pharmaceutically acceptable salt thereof.

Some embodiments include a compound of Formula 1, which is further represented by Formula 2:

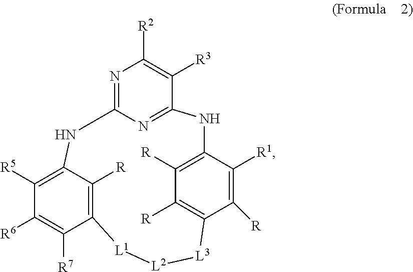

or a pharmaceutically acceptable salt thereof.

Some embodiments include a pharmaceutical composition a dosage form, and/or a medicament comprising a therapeutically effective amount of a subject compound, such as a compound of Formula 1, 1a, 1b, 1c, 1d, 1e, 1f, 1g, 1h, or 2, with at least one pharmaceutically acceptable carrier, referred to herein as a subject pharmaceutical composition. A subject pharmaceutical composition can optionally contain additional excipients.

Some embodiments include a method of selectively inhibiting the kinase activities in a mammal in need thereof, comprising administering to the mammal a therapeutically effective amount of a subject compound described herein, such as a compound of Formula 1, 1a, 1b, 1c, 1d, 1e, 1f, 1g, 1h, or 2, or a pharmaceutically acceptable salt thereof.

Some embodiments include a method of treating cancer and other diseases, conditions, or disorders, which respond to the inhibition of epidermal growth factor receptor (EGFR), anaplastic lymphoma kinase (ALK) activity, or a combination thereof, comprising administering a subject compound described herein, or a pharmaceutically acceptable salt thereof, to a mammal in need thereof.

Some embodiments include use of a subject compound described herein, or a pharmaceutically acceptable salt thereof, in the manufacture of a medicament for the treatment of cancer and other diseases, conditions, or disorders, which respond to the inhibition of epidermal growth factor receptor (EGFR), anaplastic lymphoma kinase (ALK) activity, or a combination thereof.

Some embodiments include a product kit comprising a subject pharmaceutical composition, optionally in the form of a dosage form, and a label describing how to administer the subject pharmaceutical composition to a mammal for the treatment of cancer and other diseases, conditions, or disorders, which respond to the inhibition of epidermal growth factor receptor (EGFR), anaplastic lymphoma kinase (ALK) activity, or a combination thereof.

Some embodiments include a process for making a pharmaceutical composition comprising combining a subject compound and at least one pharmaceutically acceptable carrier.

### BRIEF DESCRIPTION OF THE DRAWINGS

FIG. 1 depicts the tumor volumes over the time after the start of treatment with compound A29 with various dose amounts and vehicle control in mice in a BaF3-EGFR-Del19/T790M/C797S Cell Transplant Xenograft (CTX) Model.

### DETAILED DESCRIPTION

Unless otherwise indicated, any reference to a compound herein by structure, name, or any other means, includes pharmaceutically acceptable salts, such as sodium, potassium, and ammonium salts; prodrugs, such as ester prodrugs; alternate solid forms, such as polymorphs, solvates, hydrates, etc.; tautomers; or any other chemical species that may rapidly convert to a compound described herein under conditions in which the compounds are used as described herein.

If stereochemistry is not indicated, a name or structural depiction described herein includes any stereoisomer or any mixture of stereoisomers.

In some embodiments, a compound of Formula 1 is an R-enantiomer. In some embodiments, a compound of Formula 1 is an S-enantiomer.

A hydrogen atom in any position of a compound of Formula 1 may be replaced by a deuterium. In some embodiments, a compound of Formula 1 contains a deuterium atom. In some embodiment, a compound of Formula 1 contains multiple deuterium atoms. In some embodiments, a composition comprises a compound of Formula 1 containing deuterium at greater than natural abundance, e.g. at least 10% or at least 50% greater than natural abundance.

Unless otherwise indicated, when a compound or chemical structural feature such as aryl is referred to as being “optionally substituted,” it includes a feature that has no substituents (i.e. unsubstituted), or a feature that is “substituted,” meaning that the feature has one or more substituents. The term “substituent” is broad, and includes a moiety that occupies a position normally occupied by one or more hydrogen atoms attached to a parent compound or structural feature. In some embodiments, a substituent may be an ordinary organic moiety known in the art, which may have a molecular weight (e.g. the sum of the atomic masses of the atoms of the substituent) of 15 g/mol to 50 g/mol, 15 g/mol to 100 g/mol, 15 g/mol to 150 g/mol, 15 g/mol to 200 g/mol, 15 g/mol to 300 g/mol, or 15 g/mol to 500 g/mol. In some embodiments, a substituent comprises, or consists of: 0-30, 0-20, 0-10, or 0-5 carbon atoms; and 0-30, 0-20, 0-10, or 0-5 heteroatoms, wherein each heteroatom may independently be: N, O, S, P, Si, F, Cl, Br, or I; provided that the substituent includes one C, N, O, S, P, Si, F, Cl, Br, or I atom, wherein N or S can be oxidized. Examples of substituents include, but are not limited to, alkyl, alkenyl, alkynyl, heteroalkyl, heteroalkenyl, heteroalkynyl, aryl, heteroaryl, hydroxy, alkoxy, aryloxy, acyl, acyloxy, alkylcarboxylate, thiol, alkylthio, cyano, halo, thiocarbonyl, O-carbamyl, N-carbamyl, O-thiocarbamyl, N-thiocarbamyl, C-amido, N-amido, S-sulfonamido, N-sulfonamido, isocyanato, thiocyanato, isothiocyanato, nitro, silyl, sulfenyl, sulfinyl, sulfonyl, haloalkyl, haloalkoxyl, trihalomethanesulfonyl, trihalomethanesulfonamido, amino, phosphonic acid, etc. In some embodiments, the substituents include alkyl, alkenyl, alkynyl, —NR^{A}R^{B}, —OR^{A}, S—R^{A}, aryl, heteroaryl, heterocyclyl, hydroxy, alkoxy, aryloxy, —C(O)—R^{A}, R^{A}—C(O)O-alkylcarboxylate, —SH, cyano, halogen, —C(═S)—R^{A}, —OC(O)—NR^{A}R^{B}, R^{A}—OC(O)—N(R^{A})—, —OC(═S)—NR^{A}R^{B}, R^{A}—OC(═S)—N(R^{A})—, —C(O)NR^{A}R^{B}, R^{A}—C(O)N(R^{A})—, (R^{A}R^{B})N—S(O)₂—, —N(R^{A})—S(O)₂—R^{A}, nitro, R^{A}—S(═O)—, S(O)₂—R^{A}, haloalkyl, haloalkoxyl, —S(O)₂C(X′)₃ wherein X′ is halogen, —N(R^{A})S(O)₂C(X′)₃ wherein X′ is halogen, amino, —N(R^{A})C(O)-heteroaryl, —N(R^{A})C(O)-heterocyclyl, —C(O)N(R^{A})-heteroaryl, —C(O)N(R^{A})-heterocyclyl, or a combination thereof.

For convenience, the term “molecular weight” is used with respect to a moiety or part of a molecule to indicate the sum of the atomic masses of the atoms in the moiety or part of a molecule, even though it may not be a complete molecule.

The structures associated with some of the chemical names referred to herein are depicted below. These structures may be unsubstituted, as shown below, or substituted with a substituent that may independently be in any position normally occupied by a hydrogen atom when the structure is unsubstituted. Unless a point of attachment is indicated by [symbol] , attachment may occur at any position normally occupied by a hydrogen atom.

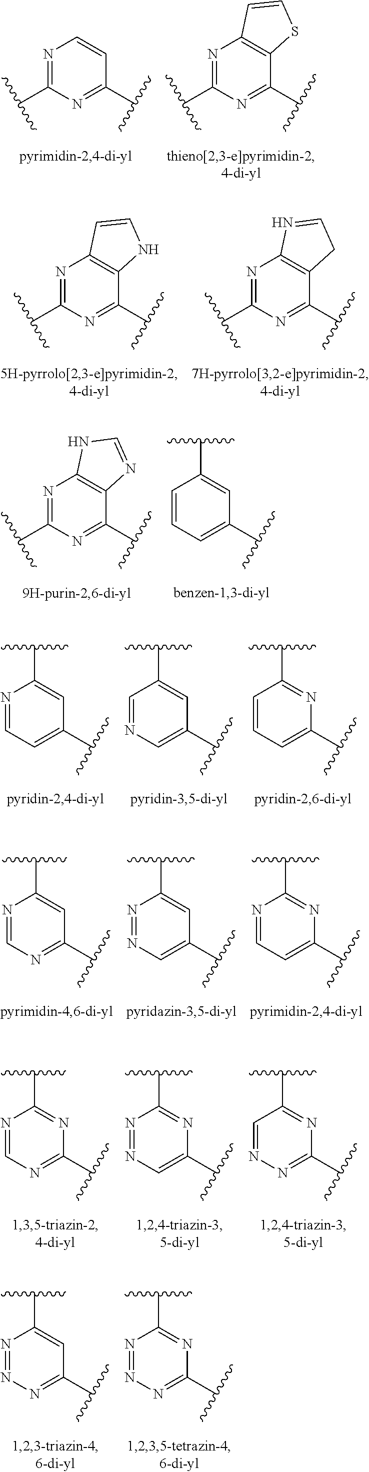

With respect to any relevant structural representation, such as Formula 1, Ring A is an optionally substituted 6-membered aromatic heterocyclic ring or an optionally substituted 9-membered fused aromatic bicyclic heterocyclic ring. In some embodiments, any or each of the substituents of Ring A may have a molecular weight of 15 g/mol to 50 g/mol, 100 g/mol, or 300 g/mol. Potential substituents of Ring A may include halo, such as F, Cl, Br, I; hydrocarbyl, such as methyl, C₂ alkyl, C₂ alkenyl, C₂ alkynyl, C₃ alkyl, C₃ cycloalkyl, C₃ alkenyl, C₃ alkynyl, C₄ alkyl, C₄ cycloalkyl, C₄ alkenyl, C₄ alkynyl, C₅ alkyl, C₅ cycloalkyl, C₅ alkenyl, C₅ alkynyl, C₆ alkyl, C₆ cycloalkyl, C₆ alkenyl, C₆ alkynyl, phenyl, etc.; C₂N₀₋₁O₀₋₂F₀₋₃H₀₋₄; C₂N₀₋₁O₀₋₃F₀₋₅H₀₋₆; C₃N₀₋₁O₀₋₃F₀₋₇H₀₋₈; C₄N₀₋₁O₀₋₃F₀₋₉H₀₋₁₀; C₅N₀₋₁O₀₋₃F₀₋₁₁H₀₋₁₂; C₆N₀₋₁O₀₋₃F₀₋₁₃H₀₋₁₄; etc. In some embodiments, Ring A is optionally substituted 6-membered aromatic heterocyclic ring having 0, 1, 2, or 3 substituents, such as pyrimidin-2,4-di-yl having 1 or 2 substituents substituted with F, Cl, Br, C₁₋₆ alkyl, —CO₂H, —CN, CO C₁₋₆ alkyl, —C(O)O—C₁₋₆-alkyl, —C₁₋₆ alkyl-OH, OH, NH₂, etc. In some embodiments, Ring A is optionally substituted pyrimidin-di-yl. In some embodiments, Ring A is optionally substituted pyrimidin-2,4-di-yl. In some embodiments, Ring A is unsubstituted pyrimidin-2,4-di-yl. In some embodiments, Ring A is pyrimidin-2,4-di-yl having 2 substituents. In some embodiments, Ring A is pyrimidin-2,4-di-yl having 1 substituent. In some embodiments, Ring A is 5-fluoro-pyrimidine-2,4-di-yl. In some embodiments, Ring A is optionally substituted 5-chloro-pyrimidine-2,4-di-yl. In some embodiments, Ring A is optionally substituted 5-chloro-pyrimidine-2,4-di-yl. In some embodiments, Ring A is optionally substituted 5-bromo-pyrimidine-2,4-di-yl. In some embodiments, Ring A is optionally substituted 5-trifluoromethyl-pyrimidine-2,4-di-yl. In some embodiments, Ring A is optionally substituted 5-chloro-pyrimidine-2,4-di-yl.

With respect to Formula 1, in some embodiments, Ring A is represented by Formula A1, A2, A3, A4, or A5:

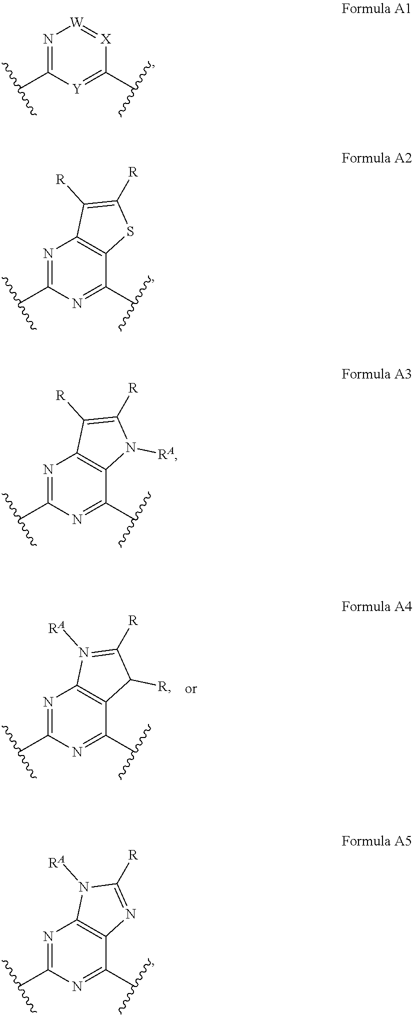

wherein each right side of the above structures is directly attached to Z in Formula 1, and W is N or CR²; X is N or CR³; and Y is N or CR⁴. In some embodiments, W is CR². In some embodiments, X is CR³. In some embodiments, Y is N.

With respect to Formula 1, in some embodiments, Ring A is represented by Formula A6:

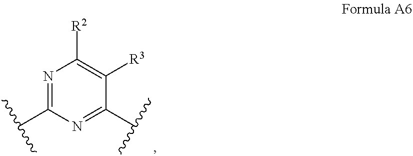

wherein each right side of the above structures is directly attached to Z in Formula 1.

For example, Ring A1-Z is:

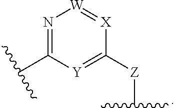

With respect to any relevant structural representation, such as Formula A1 wherein W is CR², Formula A6, or Formula 2, R² is H or any substituent, such as R^{A}, F, Cl, Br, I, CN, —OR^{A}, CF₃, —NO₂, —NR^{A}R^{B}, —COR^{A}, —CO₂R^{A}, —OCOR^{A}, —NR^{A}COR^{B}, —CONR^{A}R^{B}, or —NR^{A}C(O)O—R^{A}, etc. Some of the structures with attachment points are shown below. In some embodiments, R² may be H; F; Cl; CN; CF₃; OH; NH₂; C₁₋₆ alkyl, such as methyl, ethyl, any one of the propyl isomers (e.g. n-propyl and isopropyl), cyclopropyl, any one of the butyl isomers, any one of the cyclobutyl isomers (e.g. cyclobutyl and methylcyclopropyl), any one of the pentyl isomers, any one of the cyclopentyl isomers, any one of the hexyl isomers, and any one of the cyclohexyl isomers, etc.; or C₁₋₆ alkoxy, such as —O-methyl, —O-ethyl, any one of the isomers of —O-propyl, —O-cyclopropyl, any one of the isomers of —O-butyl, any one of the isomers of —O-cyclobutyl, any one of the isomers of —O-pentyl, any one of the isomers of —O-cyclopentyl, any one of the isomers of —O-hexyl, any one of the isomers of —O-cyclohexyl, etc. In some embodiments, R² may be H, F, or Cl. In some embodiments, R² may be H. In some embodiments, R² is F.

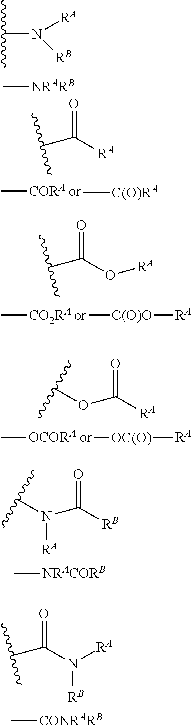

With respect to any relevant structural representation, such as formula A3, A4, or A5, each R^{A} may independently be H, or C₁₋₁₂ hydrocarbyl, such as C₁₋₁₂ alkyl, C₁₋₁₂ alkenyl, C₁₋₁₂ alkynyl, phenyl, etc., including: linear or branched alkyl having a formula CₐH₂ₐ₊₁, or cycloalkyl having a formula CₐH₂ₐ₋₁, wherein a is 1, 2, 3, 4, 5, 6, 7, 8, 9, 10, 11, or 12, such as linear or branched alkyl with a formula: CH₃, C₂H₅, C₃H₇, C₄H₉, C₅H₁₁, C₆H₁₃, C₇H₁₅, C₈H₁₇, C₉H₁₉, C₁₀H₂₁, etc., or cycloalkyl with a formula: C₃H₅, C₄H₇, C₅H₉, C₆H₁₁, C₇H₁₃, C₈H₁₅, C₉H₁₇, C₁₀H₁₉, etc. In some embodiments, R^{A} may be H or C₁₋₆ alkyl. In some embodiments, R^{A} may be H or C₁₋₃ alkyl. In some embodiments, R^{A} may be H or CH₃. In some embodiments, R^{A} may be H.

With respect to any relevant structural representation, each R^{B} may independently be H, or C₁₋₁₂ hydrocarbyl, such as C₁₋₁₂ alkyl, C₁₋₁₂ alkenyl, C₁₋₁₂ alkynyl, phenyl, etc., including: linear or branched alkyl having a formula CₐH₂ₐ₊₁, or cycloalkyl having a formula CₐH₂ₐ₋₁, wherein a is 1, 2, 3, 4, 5, 6, 7, 8, 9, 10, 11, or 12, such as linear or branched alkyl with a formula: CH₃, C₂H₅, C₃H₇, C₄H₉, C₅H₁₁, C₆H₁₃, C₇H₁₅, C₈H₁₇, C₉H₁₉, C₁₀H₂₁, etc., or cycloalkyl with a formula: C₃H₅, C₄H₇, C₅H₉, C₆H₁₁, C₇H₁₃, C₈H₁₅, C₉H₁₇, C₁₀H₁₉, etc. In some embodiments, R^{B} may be H or C₁₋₃ alkyl. In some embodiments, R^{B} may be H or CH₃. In some embodiments, R^{B} may be H.

With respect to any relevant structural representation, such as Formula A1 wherein X is CR³, formula A6, or Formula 2, R³ is H or any substituent, such as R^{A}, F, Cl, Br, I, CN, —OR^{A}, CF₃, —NO₂, —NR^{A}R^{B}, —COR^{A}, —CO₂R^{A}, —OCOR^{A}, —NR^{A}COR^{B}, —CONR^{A}R^{B}, or —NR^{A}C(O)OR^{A}, etc. In some embodiments, R³ may be H, F, Cl, CN, CF₃, OH, NH₂, C₁₋₆ alkyl, or C₁₋₆ alkoxy. In some embodiments, R³ may be H, F, Cl, Br, or CF₃. In some embodiments, R³ may be H. In some embodiments, R³ is F. In some embodiments, R³ is Cl. In some embodiments, R³ is Br. In some embodiments, R³ is CF₃. In some embodiments, R² and R³ may connect and together with Ring A to form a fused ring system.

With respect to any relevant structural representation, such as Formula A1 wherein Y is CR⁴, R⁴ is H or any substituent, such as R^{A}, F, Cl, Br, I, CN, —OR^{A}, CF₃, —NO₂, —NR^{A}R^{B}, —COR^{A}, —CO₂R^{A}, —OCOR^{A}, —NR^{A}COR^{B}, —CONR^{A}R^{B}, or —NR^{A}C(O)OR^{A}, etc. In some embodiments, R⁴ may be H, F, Cl, CN, CF₃, OH, NH₂, C₁₋₆ alkyl, or C₁₋₆ alkoxy. In some embodiments, R⁴ may be H, F, or Cl. In some embodiments, R⁴ may be H. In some embodiments, R⁴ is F.

With respect to any relevant structural representation, such as Formula A2, A3, A4, or A5, R is H or any substituent, such as R^{A}, F, Cl, Br, I, CN, —OR^{A}, CF₃, —NO₂, —NR^{A}R^{B}, —COR^{A}, —CO₂R^{A}, —OCOR^{A}, —NR^{A}COR^{B}, —CONR^{A}R^{B}, or —NR^{A}C(O)OR^{A}, etc. In some embodiments, R may be H, F, Cl, CN, CF₃, OH, NH₂, C₁₋₆ alkyl, or C₁₋₆ alkoxy. In some embodiments, R may be H, F, or Cl. In some embodiments, R may be H. In some embodiments, R is F.

With respect to any relevant structural representation, such as Formula 1, in some embodiments Z is NR^{A}, such as NH.

With respect to any relevant structural representation, such as Formula 1, Ring B is an optionally substituted 5- or 6-membered aromatic ring, or an optionally substituted 10- or 13-membered fused bicyclic ring containing one 5- or 6-membered aromatic ring and one 5, 6, or 7-membered saturated ring. In some embodiments, any or each of the substituents of Ring B may have a molecular weight of 15 g/mol to 50 g/mol, 100 g/mol, or 300 g/mol. Potential substituents of Ring B may include halo, such as F, Cl, Br, or I; hydrocarbyl, such as methyl, C₂ alkyl, C₂ alkenyl, C₂ alkynyl, C₃ alkyl, C₃ cycloalkyl, C₃ alkenyl, C₃ alkynyl, C₄ alkyl, C₄ cycloalkyl, C₄ alkenyl, C₄ alkynyl, C₅ alkyl, C₅ cycloalkyl, C₅ alkenyl, C₅ alkynyl, C₆ alkyl, C₆ cycloalkyl, C₆ alkenyl, C₆ alkynyl, or phenyl, etc.; CN₀₋₁O₀₋₂F₀₋₃H₀₋₄; C₂N₀₋₁O₀₋₃F₀₋₅H₀₋₆; C₃N₀₋₁O₀₋₃F₀₋₇H₀₋₈; C₄N₀₋₁O₀₋₃F₀₋₉H₀₋₁₀; C₅N₀₋₁O₀₋₃F₀₋₁₁H₀₋₁₂; or C₆N₀₋₁O₀₋₃F₀₋₁₃H₀₋₁₄; etc. In some embodiments, Ring B is optionally substituted 1,2,4,5-tetrazin-3,6-di-yl. In some embodiments, Ring B is optionally substituted 1,2,4-triazin-3,6-di-yl. In some embodiments, Ring B is optionally substituted pyridazin-3,6-di-yl. In some embodiments, Ring B is optionally substituted pyrimidin-2,5-di-yl. In some embodiments, Ring B is optionally substituted pyrazin-2,5-di-yl. In some embodiments, Ring B is optionally substituted pyridin-2,5-di-yl. In some embodiments, Ring B is optionally substituted benzene-di-yl, such as 1,3-benzen-di-yl having 0, 1, 2, 3, or 4 substituents, such as 1,3-benzen-di-yl substituted with F, Cl, Br, I, C₁₋₆ alkyl, —CO₂H, —CN, —CO—C₁₋₆-alkyl, —C(O)O—C₁₋₆-alkyl, —C₁₋₆ alkyl-OH, OH, NH₂, etc. In some embodiments, Ring B is 1,3-benzen-di-yl having 2 substituents. In some embodiments, Ring B is 1,3-benzen-di-yl having 1 substituent. In some embodiments, Ring B is 1,3-benzen-di-yl having an alkoxy (such as methoxy) substituent. In some embodiments, Ring B is unsubstituted 1,3-benzen-di-yl.

In some embodiments, Ring B is represented by formula B1, B2, B3, or B4:

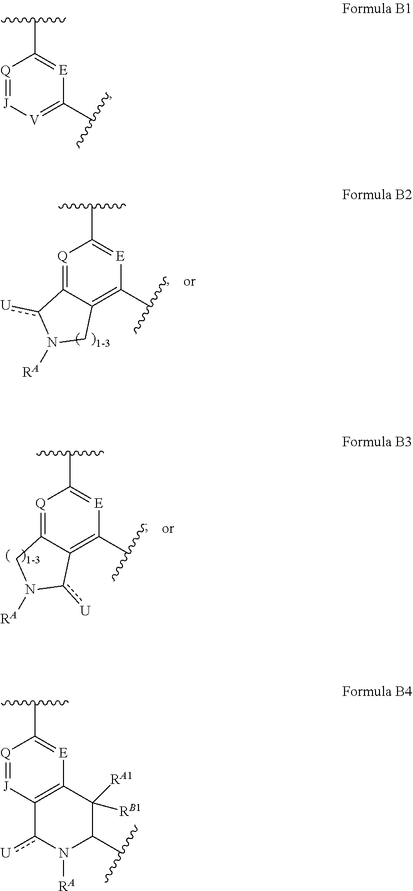

wherein L¹-L² of Formula 1 is an optionally substituted C₁₋₈ alkylene and L³ is O, wherein each top side of the above structures is linked to Ring A via NH in the Formula of claim 1; each of the above structures of Ring B is optionally substituted; each E is independently CR, NR^{A}, O, or S; each Q is independently CR⁵, NR^{A}, O, or S; each J is independently a bond, CR⁶, or N; U is O or H₂; V is CR⁷, NR^{A}, O, or S. In some embodiments, E is CR. In some embodiments, Q is CR5. In some embodiments, J is CR⁶. In some embodiments, V is CR⁷.

In some embodiments, Ring B is represented by Formula B1. In some embodiments, Ring B is represented by formula B5:

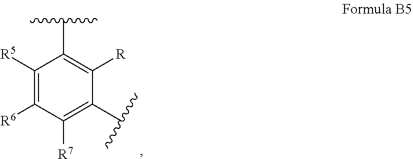

wherein the top side of the structure is linked to Ring A via NH in Formula 1.

With respect to any relevant structural representation, such as Formula B1, B2, B3, or B4 where E is CR, Formula B5, or Formula 2, R is H or any substituent, such as R^{A}, F, Cl, Br, I, CN, —OR^{A}, CF₃, —NO₂, —NR^{A}R^{B}, —COR^{A}, —CO₂R^{A}, —OCOR^{A}, —NR^{A}COR^{B}, —CONR^{A}R^{B}, or —NR^{A}C(O)OR^{A}, etc. In some embodiments, R may be H, F, Cl, CN, CF₃, OH, NH₂, C₁₋₆ alkyl, or C₁₋₆ alkoxy. In some embodiments, R may be H, F, Cl, or —OR^{A}. In some embodiments, R may be H. In some embodiments, R may be F.

With respect to any relevant structural representation, such as Formula B1, B2, B3, or B4 where Q is CR⁵, Formula B5, or Formula 2, R⁵ is H or any substituent, such as R^{A}, F, Cl, Br, I, CN, —OR^{A}, CF₃, —NO₂, —NR^{A}R^{B}, —COR^{A}, —CO₂R^{A}, —OCOR^{A}, —NR^{A}COR^{B}, —CONR^{A}R^{B}, or —NR^{A}C(O)OR^{A}, etc. In some embodiments, R⁵ may be H, F, Cl, CN, CF₃, OH, NH₂, C₁₋₆ alkyl, or C₁₋₆ alkoxy. In some embodiments, R⁵ may be H, F, Cl, or —OR^{A}. In some embodiments, R⁵ may be H. In some embodiments, R⁵ is F. In some embodiments, R⁵ may be —OCH₃, —OCH₂CH₃, or —OC(CH)(CH₃)₂. In some embodiments, R⁵ is —OCH₂CH₃. In some embodiments, R⁵ is —OC(CH)(CH₃)₂. In some embodiments, R⁵ is —OCH₃.

With respect to any relevant structural representation, such as Formula B1 or B4 where J is CR⁶, Formula B5, or Formula 2, R⁶ is H or any substituent, such as R^{A}, F, Cl, Br, I, CN, —OR^{A}, CF₃, —NO₂, —NR^{A}R^{B}, —COR^{A}, —CO₂R^{A}, —OCOR^{A}, —NR^{A}COR^{B}, —CONR^{A}R^{B}, or —NR^{A}C(O)OR^{A}, etc. In some embodiments, R⁶ may be H, F, Cl, CN, CF₃, OH, NH₂, C₁₋₆ alkyl, or C₁₋₆ alkoxy. In some embodiments, R⁶ may be H, F, or Cl. In some embodiments, R⁶ may be H. In some embodiments, R⁶ is F. In some embodiments, R⁵ and R⁶ may connect and together with Ring B to form a fused ring system;

With respect to any relevant structural representation, such as Formula B1 where V is CR², Formula B5, or Formula 2, R⁷ is H or any substituent, such as R^{A}, F, Cl, Br, I, CN, —OR^{A}, CF₃, —NO₂, —NR^{A}R^{B}, —COR^{A}, —CO₂R^{A}, —OCOR^{A}, —NR^{A}COR^{B}, —CONR^{A}R^{B}, or —NR^{A}C(O)OR^{A}, etc. In some embodiments, R⁷ may be H, F, Cl, Br, I, NR^{A}R^{B}, —NR^{A} (CR^{A1}R^{B1})₁₋₃ NR^{A}R^{B}, C₁₋₆ hydrocarbyl, —OH, —CN, —NO₂, —O—C₁₋₆ alkyl, or —C(O)O—C₁₋₆ alkyl, —NR^{A}S(O)₂R^{B}, —S(O)₂NR^{A}R^{B}, —C(O)NR^{A}R^{B}, —NR^{A}C(O)R^{A}R^{B}, —NR^{A}C(O)NR^{A}R^{B}, OC(O)NR^{A}R^{B}, CR^{A1}R^{B1}C(O)NR^{A}R^{B}, an optionally substituted 5- or 6-membered saturated mono-cyclic ring containing 1 or 2 ring N atoms and 0 to 1 ring 0 atom, or an optionally substituted 8 to 12 membered saturated bicyclic ring system containing 2 to 3 ring N atoms and 0 to 1 ring O atom; and wherein each R², each R³, each R⁴, each R⁵, each R⁶, and each R⁷ are independently optionally halogenated. In some embodiments, R⁷ may be —NR^{A}R^{B}. In In some embodiments, R⁷ may be:

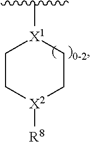

wherein the structure is optionally substituted with 0, 1, 2, 3, 4, 5, 6, 7, 8, 9, or 10 substituents, X¹ and X² are independently CH or N, and R⁸ is H or any substituent, such as R^{A}, F, Cl, Br, I, CN, —OR^{A} CF₃, —NO₂, —NR^{A}R^{B}, —COR^{A}, —CO₂R^{A}, —OCOR^{A}, —NR^{A}COR^{B}, —CONR^{A}R^{B}, or —NR^{A}C(O)OR^{A}, etc. In some embodiments, R⁸ may be H, F, Cl, CN, CF₃, OH, NH₂, C₁₋₆ alkyl, or C₁₋₆ alkoxy. In some embodiments, R⁸ may be H, F, or Cl. In some embodiments, R⁸ may be H.

In some embodiments, R⁷ is:

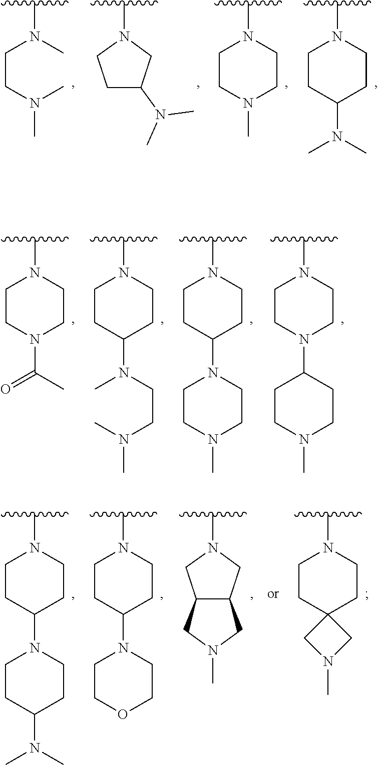

wherein each structure is optionally substituted.

With respect to any relevant structural representation, such as Formula B4, R^{A1} and R^{B1} are independently H or any substituent, such as R^{A}, F, Cl, Br, I, CN, —OR^{A}, CF₃, —NR^{A}R^{B}, —COR^{A}, —CO₂R^{A}, —OCOR^{A}, —NR^{A}COR^{B}, or —CONR^{A}R^{B}, or —NR^{A}C(O)OR^{A}, etc. In some embodiments, R^{A1} and R^{B1} may be independently H, F, Cl, CN, CF₃, OH, NH₂, C₁₋₆ alkyl, or C₁₋₆ alkoxy. In some embodiments, R^{A1} and R^{B1} may be independently H, F, or Cl. In some embodiments, R^{A1} and R^{B1} may be independently H. In some embodiments, R^{A1} and R^{B1} may be independently C₁₋₆ hydrocarbyl.

With respect to any relevant structural representation, such as Formula 1, 1a, 1b, 1c, 1d, 1e, 1f, 1g, or 1h, wherein

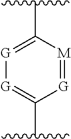

is 1,2,4,5-tetrazin-3,6-di-yl, an optionally substituted 1,2,4-triazin-3,6-di-yl, an optionally substituted pyridazin-3,6-di-yl, an optionally substituted pyrimidin-2,5-di-yl, an optionally substituted pyrazin-2,5-di-yl, an optionally substituted pyridin-2,5-di-yl, or an optionally substituted 1,4-benzen-di-yl; and wherein M is CR¹ or N; and each G is independently CR or N. In some embodiments, G may be CR. In some embodiments, each G may be independently CH. In some embodiments, each G is CH. In some embodiments, M is CR¹. In some embodiments, each G is CH, and M is CR¹.

With respect to any relevant structural representation, such as Formula 1, 1a, 1b, 1c, 1d, 1e, 1f, 1g, or 1h wherein G is CR, or formula 2, each R is independently H or any substituent, such as R^{A}, F, Cl, Br, I, CN, —OR^{A} CF₃, —NO₂, —NR^{A}R^{B}, —COR^{A}, —CO₂R^{A}, —OCOR^{A}, —NR^{A}COR^{B}, —CONR^{A}R^{B} or —NR^{A}C(O)OR^{A}, etc. In some embodiments, each R may be independently H, F, Cl, CN, CF₃, OH, NH₂, C₁₋₆ alkyl, or C₁₋₆ alkoxy. In some embodiments, each R may be independently H, F, Cl, or —OR^{A}. In some embodiments, each R may be independently H. In some embodiments, each R may be independently F.

With respect to any relevant structural representation, such as Formula 1, 1a, 1b, 1c, 1d, 1e, 1f, 1g, 1h, wherein M is CR¹, or Formula 2, R¹ is H or any substituent, such as R^{A}, F, Cl, Br, I, CN, OR^{A}, CF₃, NR^{A}R^{B}, COR^{A}, CO_{Z}R^{A}, OCOR^{A}, NR^{A}COR^{B}, or CONR^{A}R^{B}, NR^{A}C(O)OR^{A}, —S(O)₁₋₂R^{A}; P(O)R^{A}R^{B}, NR^{A}S(O)₂R^{B}, S(O)₂NR^{A}R^{B}, etc. In some embodiments, R¹ is H, F, Cl, Br, I, —NR^{A}R^{B}, C₁₋₆ hydrocarbyl, —OH, —CN, —NO₂, —O—C₁₋₆ alkyl, —C(O)O—C₁₋₆ alkyl, —S(O)₁₋₂R^{A}; —P(O)R^{A}R^{B}, —NR^{A}S(O)₂R^{B}, —S(O)₂NR^{A}R^{B}, —C(O)NR^{A}R^{B}, —NR^{A}C(O)R^{A}R^{B}. In some embodiments, R¹ is —C₀₋₃H₁₋₇N₀₋₁—S(O)₂—C₁₋₄H₃₋₁₀. In some embodiments, R¹ is —P(O)(C₁₋₅H₃₋₁₁)(C₁₋₄H₃₋₉). In some embodiments, R¹ is:

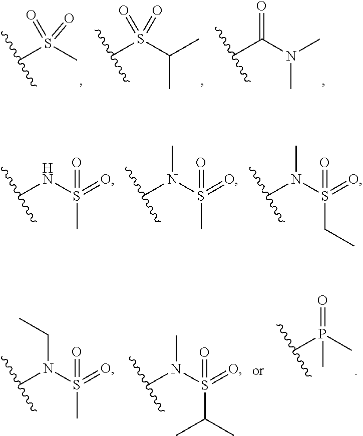

With respect to any relevant structural representation, such as Formula 1, 1a, 1c, 1d, 1e, 1f, 1g, 1h, or 2, wherein L¹ and L³ are independently a covalent bond, O, NR^{A}, S(O)₀₋₂, CR^{A1}R^{B1}, CR^{A1}═CR^{B1}, —C(O)NR^{A}—, —NR^{A}(CO)—, S(O)₁₋₂NR^{A}, or NR^{A}C(O)NR^{B}. In some embodiments, 12 is a covalent bond. In some embodiments, L¹ is —NR^{A}C(O)—. In some embodiments, L¹ is —NHC(O)—. In some embodiments, L³ is O. In some embodiments, L³ is a covalent bond.

With respect to any relevant structural representation, such as Formula 1, 1a, 1c, 1d, 1e, 1f, 1g, 1h, or 2, wherein L² is an optionally substituted C₁₋₁₂ alkylene, Cₘ alkylene-C(O)NR^{A}—Cₙ alkylene, Cₘ alkylene-NR^{A}(CO)—Cₙ alkylene, or Cₘ alkylene-O—Cₙ alkylene, wherein m is 1 to 12, n is 1 to 12, provided that the sum of m and n is no more than 12, wherein L² has, as chemically appropriate, 0, 1, 2, 3, 4, 5, 6, 7, 8, 9, 10, 11, or 12 substituents, and the substituents of L² are independently F, Cl, Br, I, OH, ═O, C₁₋₆ alkyl, or C₁₋₆ cycloalkyl. In some embodiments, L² is an optionally substituted C₁₋₆ alkylene, Cₘ alkylene-C(O)NR^{A}—Cₙ alkylene, or Cₘ alkylene-NR^{A}(CO)—Cₙ alkylene, wherein m is 1 to 6, n is 1 to 6, provided that the sum of m and n is no more than 6. In some embodiments, L² is an optionally substituted C₁₋₆ alkylene, Cₘ alkylene-C(O)NH—Cₙ alkylene, or Cₘ alkylene-NH(CO)—Cₙ alkylene, wherein m is 1 to 6, n is 1 to 6, provided that the sum of m and n is no more than 6. In some embodiments, L² is an optionally substituted C₃₋₆ alkylene. In some embodiments, L² is unsubstituted —(CH₂)₆—. In some embodiments, L² is unsubstituted —(CH₂)₅—.

In some embodiments, L¹-L²-L³ is represented by the empirical formula C₁₋₁₂N₀₋₁O₀₋₂H₂₋₂₆. In some embodiments, L¹-L²-L³ is represented by the empirical formula C₃₋₈N₀₋₁O₀₋₂H₆₋₁₈. In some embodiments, L¹-L²-L³ is represented by the empirical formula C₁₋₁₂O₀₋₂H₂₋₂₄ (e.g. where the number of H atoms is double the number of hydrogen atoms, such as OCH₂, OC₂H₄, etc.). In some embodiments, L¹-L²-L³ is represented by the empirical formula C₃₋₈O₀₋₂H₆₋₁₆.

Some embodiments include a compound that is optionally substituted 6-oxa-2,4-diaza-3(2,4)-pyrimidina-1(1,3),5(1,4)-dibenzenacyclononaphane, optionally substituted 6-oxa-2,4-diaza-3(2,4)-pyrimidina-1(1,3),5(1,4)-dibenzenacyclodecaphane, optionally substituted 6-oxa-2,4-diaza-3(2,4)-pyrimidina-1(1,3),5(1,4)-dibenzenacycloundecaphane, optionally substituted 6-oxa-2,4-diaza-3(2,4)-pyrimidina-1(1,3),5(1,4)-dibenzenacyclododecaphane, optionally substituted 6-oxa-2,4,11-triaza-3(2,4)-pyrimidina-1(1,3),5(1,4)-dibenzenacycloundecaphan-10-one, optionally substituted 6-oxa-2,4,12-triaza-3(2,4)-pyrimidina-1(1,3),5(1,4)-dibenzenacyclododecaphan-11-one, optionally substituted 2,4,9-triaza-3(2,4)-pyrimidina-1(1,3),5(1,4)-dibenzenacyclododecaphan-8-one, or optionally substituted 2,4,9-triaza-3(2,4)-pyrimidina-1(1,3),5(1,4)-dibenzenacyclododecaphan-10-one.

Some embodiments include one of the compounds listed in Table 1 below, wherein each structure can be optionally substituted:

TABLE 1
Compound structures, names, and their ID numbers

| Compound | | |
|---|---|---|
| ID | Structure | Name |
| A1 | 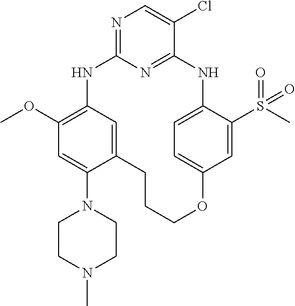 [SMILES: COc1cc(N2CCN(C)CC2)c2cc1Nc1ncc(Cl)c(n1)Nc1ccc(cc1S(C)(=O)=O)OCCC2] | 3⁵-chloro-1⁶-methoxy-1⁴-(4-methylpiperazin- 1-yl)-5²-(methylsulfonyl)-6-oxa-2,4-diaza- 3(2,4)-pyrimidina-1(1,3),5(1,4)- dibenzenacyclononaphane |
| A2 | 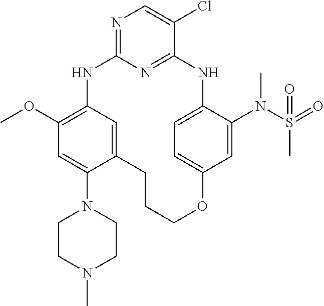 [SMILES: COc1cc(N2CCN(C)CC2)c2cc1Nc1ncc(Cl)c(n1)Nc1ccc(cc1N(C)S(C)(=O)=O)OCCC2] | N-(3⁵-chloro-1⁶-methoxy-1⁴-(4- methylpiperazin-1-yl)-6-oxa-2,4-diaza-3(2,4)- pyrimidina-1(1,3),5(1,4)- dibenzenacyclononaphane-5²-yl)-N- methylmethanesulfonamide |
| A3 | 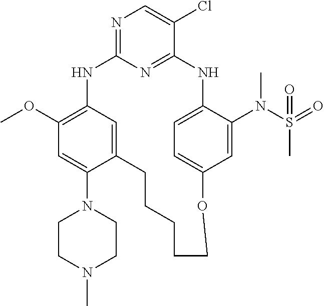 [SMILES: COc1cc(N2CCN(C)CC2)c2cc1Nc1ncc(Cl)c(n1)Nc1ccc(cc1N(C)S(C)(=O)=O)OCCCCC2] | N-(3⁵-chloro-1⁶-methoxy-1⁴-(4- methylpiperazin-1-yl)-6-oxa-2,4-diaza-3(2,4)- pyrimidina-1(1,3),5(1,4)- dibenzenacycloundecaphane-5²-yl)-N- methylmethanesulfonamide |
| A4 | 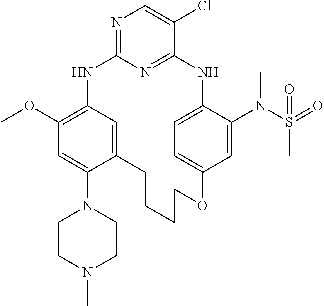 [SMILES: COc1cc(N2CCN(C)CC2)c2cc1Nc1ncc(Cl)c(n1)Nc1ccc(cc1N(C)S(C)(=O)=O)OCCCC2] | N-(3⁵-chloro-1⁶-methoxy-1⁴-(4- methylpiperazin-1-yl)-6-oxa-2,4-diaza-3(2,4)- pyrimidina-1(1,3),5(1,4)- dibenzenacyclodecaphane-5²-yl)-N- methylmethanesulfonamide |
| A5 | 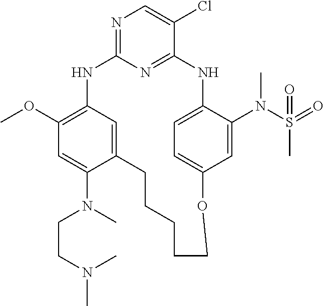 [SMILES: COc1cc(N(C)CCN(C)C)c2cc1Nc1ncc(Cl)c(n1)Nc1ccc(cc1N(C)S(C)(=O)=O)OCCCCC2] | N-(3⁵-chloro-1⁴-((2- (dimethylamino)ethyl)(methyl)amino)-1⁶- methoxy-6-oxa-2,4-diaza-3(2,4)-pyrimidina- 1(1,3),5(1,4)-dibenzenacycloundecaphane-5²- yl)-N-methylmethanesulfonamide |
| A6 | 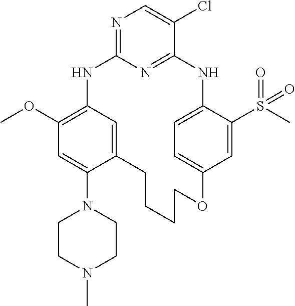 [SMILES: COc1cc(N2CCN(C)CC2)c2cc1Nc1ncc(Cl)c(n1)Nc1ccc(cc1S(C)(=O)=O)OCCCC2] | 3⁵-chloro-1⁶-methoxy-1⁴-(4-methylpiperazin- 1-yl)-5²-(methylsulfonyl)-6-oxa-2,4-diaza- 3(2,4)-pyrimidina-1(1,3),5(1,4)- dibenzenacyclodecaphane |
| A7 | 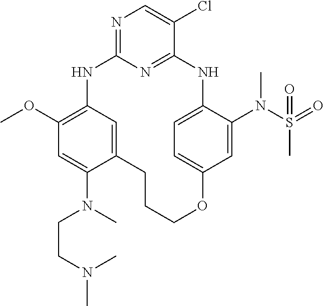 [SMILES: COc1cc(N(C)CCN(C)C)c2cc1Nc1ncc(Cl)c(n1)Nc1ccc(cc1N(C)S(C)(=O)=O)OCCC2] | N-(3⁵-chloro-1⁴-((2- (dimethylamino)ethyl)(methyl)amino)-1⁶- methoxy-6-oxa-2,4-diaza-3(2,4)-pyrimidina- 1(1,3),5(1,4)-dibenzenacyclononaphane-5²- yl)-N-methylmethanesulfonamide |
| A8 | 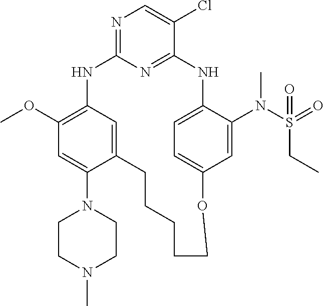 [SMILES: CCS(=O)(=O)N(C)c1cc2ccc1Nc1nc(ncc1Cl)Nc1cc(c(N3CCN(C)CC3)cc1OC)CCCCCO2] | N-(3⁵-chloro-1⁶-methoxy-1⁴-(4- methylpiperazin-1-yl)-6-oxa-2,4-diaza-3(2,4)- pyrimidina-1(1,3),5(1,4)- dibenzenacycloundecaphane-5²-yl)-N- methylethanesulfonamide |
| A9 | 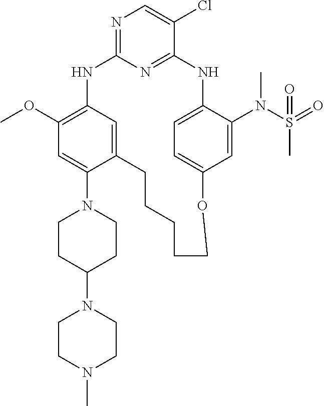 [SMILES: COc1cc(N2CCC(N3CCN(C)CC3)CC2)c2cc1Nc1ncc(Cl)c(n1)Nc1ccc(cc1N(C)S(C)(=O)=O)OCCCCC2] | N-(3⁵-chloro-1⁶-methoxy-1⁴-(4-(4- methylpiperazin-1-yl)piperidin-1-yl)-6-oxa- 2,4-diaza-3(2,4)-pyrimidina-1(1,3),5(1,4)- dibenzenacycloundecaphane-5²-yl)-N- methylmethanesulfonamide |
| A10 | 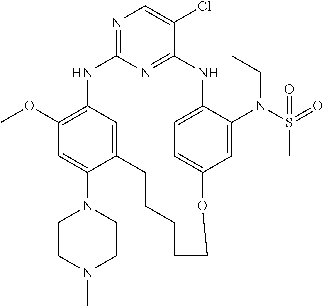 [SMILES: CCN(c1cc2ccc1Nc1nc(ncc1Cl)Nc1cc(c(N3CCN(C)CC3)cc1OC)CCCCCO2)S(C)(=O)=O] | N-(3⁵-chloro-1⁶-methoxy-1⁴-(4- methylpiperazin-1-yl)-6-oxa-2,4-diaza-3(2,4)- pyrimidina-1(1,3),5(1,4)- dibenzenacycloundecaphane-5²-yl)-N- ethylmethanesulfonamide |
| A11 | 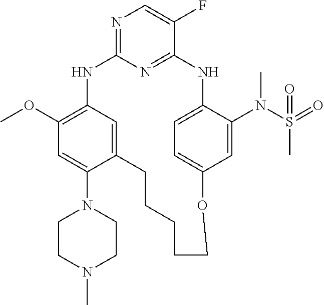 [SMILES: COc1cc(N2CCN(C)CC2)c2cc1Nc1ncc(F)c(n1)Nc1ccc(cc1N(C)S(C)(=O)=O)OCCCCC2] | N-(3⁵-fluoro-1⁶-methoxy-1⁴-(4- methylpiperazin-1-yl)-6-oxa-2,4-diaza-3(2,4)- pyrimidina-1(1,3),5(1,4)- dibenzenacycloundecaphane-5²-yl)-N- methylmethanesulfonamide |
| A12 | 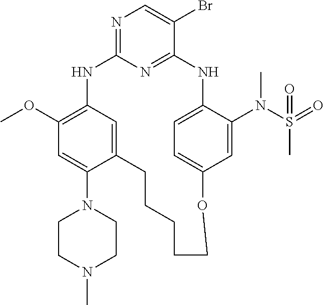 [SMILES: COc1cc(N2CCN(C)CC2)c2cc1Nc1ncc(Br)c(n1)Nc1ccc(cc1N(C)S(C)(=O)=O)OCCCCC2] | N-(3⁵-bromo-1⁶-methoxy-1⁴-(4- methylpiperazin-1-yl)-6-oxa-2,4-diaza-3(2,4)- pyrimidina-1(1,3),5(1,4)- dibenzenacycloundecaphane-5²-yl)-N- methylmethanesulfonamide |
| A13 | 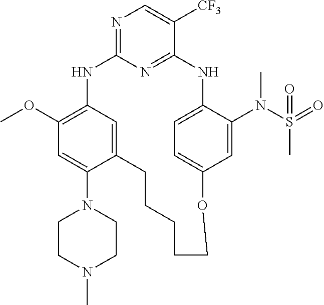 [SMILES: COc1cc(N2CCN(C)CC2)c2cc1Nc1ncc(C(F)(F)F)c(n1)Nc1ccc(cc1N(C)S(C)(=O)=O)OCCCCC2] | N-(1⁶-methoxy-1⁴-(4-methylpiperazin- 1-yl)-3⁵-(trifluoromethyl)-6-oxa-2,4-diaza- 3(2,4)-pyrimidina-1(1,3),5(1,4)- dibenzenacycloundecaphane-5²-yl)-N- methylmethanesulfonamide |
| A14 | 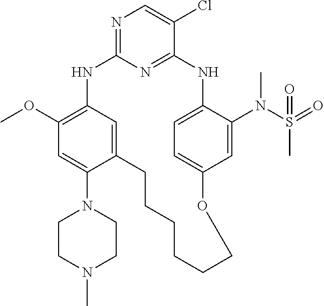 [SMILES: COc1cc(N2CCN(C)CC2)c2cc1Nc1ncc(Cl)c(n1)Nc1ccc(cc1N(C)S(C)(=O)=O)OCCCCCC2] | N-(3⁵-chloro-1⁶-methoxy-1⁴-(4- methylpiperazin-1-yl)-6-oxa-2,4-diaza-3(2,4)- pyrimidina-1(1,3),5(1,4)- dibenzenacyclododecaphane-5²-yl)-N- methylmethanesulfonamide |
| A15 | 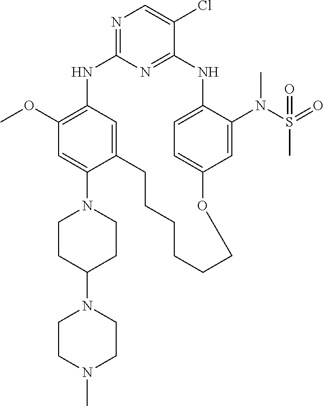 [SMILES: COc1cc(N2CCC(N3CCN(C)CC3)CC2)c2cc1Nc1ncc(Cl)c(n1)Nc1ccc(cc1N(C)S(C)(=O)=O)OCCCCCC2] | N-(3⁵-chloro-1⁶-methoxy-1⁴-(4-(4- methylpiperazin-1-yl)piperidin-1-yl)-6-oxa- 2,4-diaza-3(2,4)-pyrimidina-1(1,3),5(1,4)- dibenzenacyclododecaphane-5²-yl)-N- methylmethanesulfonamide |
| A16 | 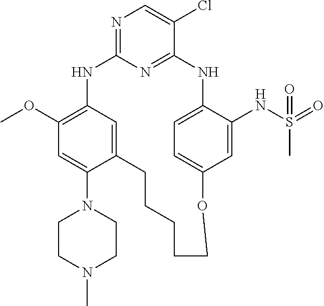 [SMILES: COc1cc(N2CCN(C)CC2)c2cc1Nc1ncc(Cl)c(n1)Nc1ccc(cc1NS(C)(=O)=O)OCCCCC2] | N-(3⁵-chloro-1⁶-methoxy-1⁴-(4- methylpiperazin-1-yl)-6-oxa-2,4-diaza-3(2,4)- pyrimidina-1(1,3),5(1,4)- dibenzenacycloundecaphane-5²- yl)methanesulfonamide |
| A17 | 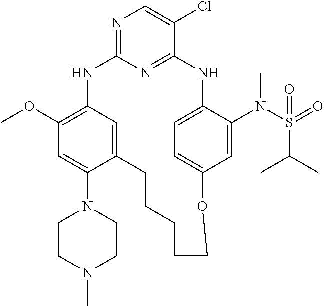 [SMILES: COc1cc(N2CCN(C)CC2)c2cc1Nc1ncc(Cl)c(n1)Nc1ccc(cc1N(C)S(=O)(=O)C(C)C)OCCCCC2] | N-(3⁵-chloro-1⁶-methoxy-1⁴-(4- methylpiperazin-1-yl)-6-oxa-2,4-diaza-3(2,4)- pyrimidina-1(1,3),5(1,4)- dibenzenacycloundecaphane-5²-yl)-N- methylpropane-2-sulfonamide |
| A18 | 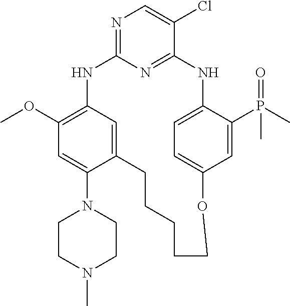 | (3⁵-chloro-1⁶-methoxy-1⁴-(4- methylpiperazin-1-yl)-6-oxa-2,4-diaza-3(2,4)- pyrimidina-1(1,3),5(1,4)- dibenzenacycloundecaphane-5²- yl)dimethylphosphine oxide |
| A19 | 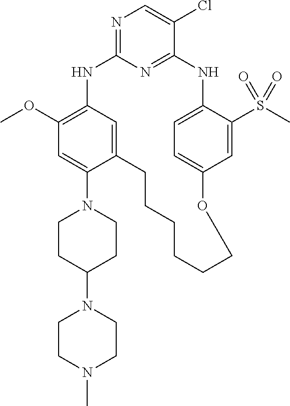 | 3⁵-chloro-1⁶-methoxy-1⁴-(4-(4- methylpiperazin-1-yl)piperidin-1-yl)-5²- (methylsulfonyl)-6-oxa-2,4-diaza-3(2,4)- pyrimidina-1(1,3),5(1,4)- dibenzenacyclododecaphane |
| A20 | 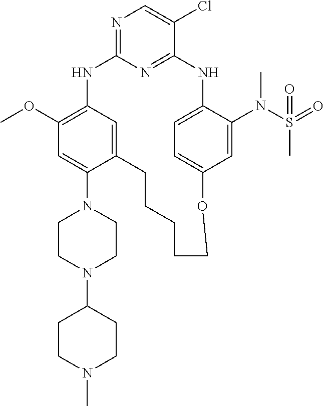 [SMILES: COc1cc(N2CCN(C3CCN(C)CC3)CC2)c2cc1Nc1ncc(Cl)c(n1)Nc1ccc(cc1N(C)S(C)(=O)=O)OCCCCC2] | N-(3⁵-chloro-1⁶-methoxy-1⁴-(4-(1- methylpiperidin-4-yl)piperazin-1-yl)-6-oxa- 2,4-diaza-3(2,4)-pyrimidina-1(1,3),5(1,4)- dibenzenacycloundecaphane-5²-yl)-N- methylmethanesulfonamide |
| A21 | 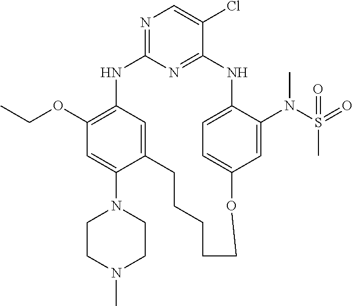 [SMILES: CCOc1cc(N2CCN(C)CC2)c2cc1Nc1ncc(Cl)c(n1)Nc1ccc(cc1N(C)S(C)(=O)=O)OCCCCC2] | N-(3⁵-chloro-1⁶-ethoxy-1⁴-(4- methylpiperazin-1-yl)-6-oxa-2,4-diaza-3(2,4)- pyrimidina-1(1,3),5(1,4)- dibenzenacycloundecaphane-5²-yl)-N- methylmethanesulfonamide |
| A22 | 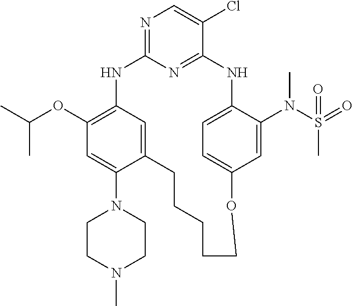 [SMILES: CC(C)Oc1cc(N2CCN(C)CC2)c2cc1Nc1ncc(Cl)c(n1)Nc1ccc(cc1N(C)S(C)(=O)=O)OCCCCC2] | N-(3⁵-chloro-1⁶-isopropoxy-1⁴-(4- methylpiperazin-1-yl)-6-oxa-2,4-diaza-3(2,4)- pyrimidina-1(1,3),5(1,4)- dibenzenacycloundecaphane-5²-yl)-N- methylmethanesulfonamide |
| A23 | 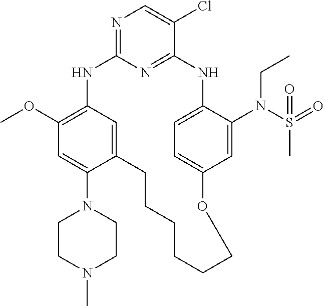 [SMILES: CCN(c1cc2ccc1Nc1nc(ncc1Cl)Nc1cc(c(N3CCN(C)CC3)cc1OC)CCCCCCO2)S(C)(=O)=O] | N-(3⁵-chloro-1⁶-methoxy-1⁴-(4- methylpiperazin-1-yl)-6-oxa-2,4-diaza-3(2,4)- pyrimidina-1(1,3),5(1,4)- dibenzenacyclododecaphane-5²-yl)-N- ethylmethanesulfonamide |
| A24 | 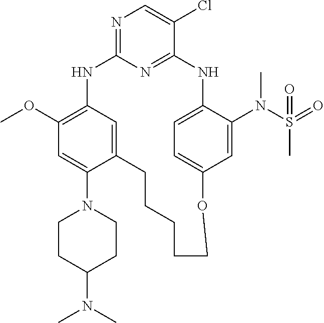 [SMILES: COc1cc(N2CCC(N(C)C)CC2)c2cc1Nc1ncc(Cl)c(n1)Nc1ccc(cc1N(C)S(C)(=O)=O)OCCCCC2] | N-(3⁵-chloro-1⁴-(4- (dimethylamino)piperidin-1-yl)-1⁶-methoxy-6- oxa-2,4-diaza-3(2,4)-pyrimidina-1(1,3),5(1,4)- dibenzenacycloundecaphane-5²-yl)-N- methylmethanesulfonamide |
| A25 | 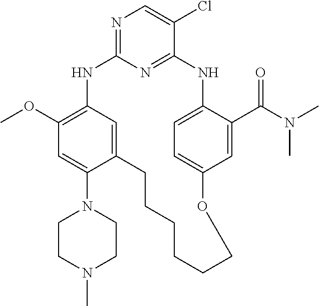 | 3⁵-chloro-1⁶-methoxy-N,N-dimethyl- 1⁴-(4-methylpiperazin-1-yl)-6-oxa-2,4-diaza- 3(2,4)-pyrimidina-1(1,3),5(1,4)- dibenzenacyclododecaphane-5²-carboxamide |
| A26 | 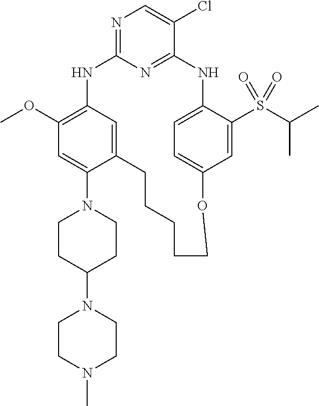 [SMILES: COc1cc(N2CCC(N3CCN(C)CC3)CC2)c2cc1Nc1ncc(Cl)c(n1)Nc1ccc(cc1S(=O)(=O)C(C)C)OCCCCC2] | 3⁵-chloro-5²-(isopropylsulfonyl)-16-methoxy- 1⁴-(4-(4-methylpiperazin-1-yl)piperidin-1-yl)- 6-oxa-2,4-diaza-3(2,4)-pyrimidina- 1(1,3),5(1,4)-dibenzenacycloundecaphane |
| A27 | 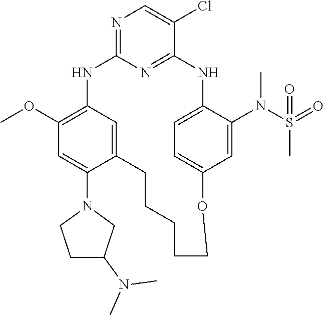 [SMILES: COc1cc(N2CCC(N(C)C)C2)c2cc1Nc1ncc(Cl)c(n1)Nc1ccc(cc1N(C)S(C)(=O)=O)OCCCCC2] | N-(3⁵-chloro-1⁴-(3- (dimethylamino)pyrrolidin-1-yl)-1⁶-methoxy- 6-oxa-2,4-diaza-3(2,4)-pyrimidina- 1(1,3),5(1,4)-dibenzenacycloundecaphane-5²- yl)-N-methylmethanesulfonamide |
| A28 | 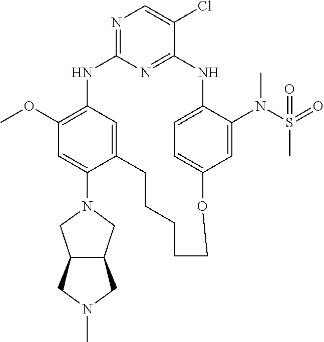 [SMILES: COc1cc(N2C[C@H]3CN(C)C[C@H]3C2)c2cc1Nc1ncc(Cl)c(n1)Nc1ccc(cc1N(C)S(C)(=O)=O)OCCCCC2] | N-(3⁵-chloro-1⁶-methoxy-1⁴-((cis-3,6)- 5-methylhexahydropyrrolo[3,4-c]pyrrol- 2(1H)-yl)-6-oxa-2,4-diaza-3(2,4)-pyrimidina- 1(1,3),5(1,4)-dibenzenacycloundecaphane-5²- yl)-N-methylmethanesulfonamide |
| A29 | 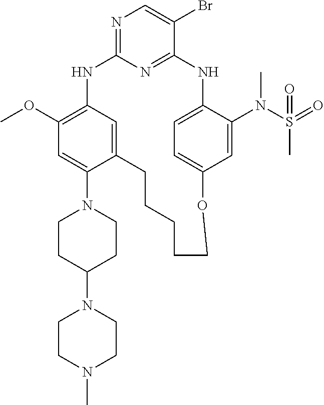 [SMILES: COc1cc(N2CCC(N3CCN(C)CC3)CC2)c2cc1Nc1ncc(Br)c(n1)Nc1ccc(cc1N(C)S(C)(=O)=O)OCCCCC2] | N-(3⁵-bromo-1⁶-methoxy-1⁴-(4-(4- methylpiperazin-1-yl)piperidin-1-yl)-6-oxa- 2,4-diaza-3(2,4)-pyrimidina-1(1,3),5(1,4)- dibenzenacycloundecaphane-5²-yl)-N- methylmethanesulfonamide |
| A30 | 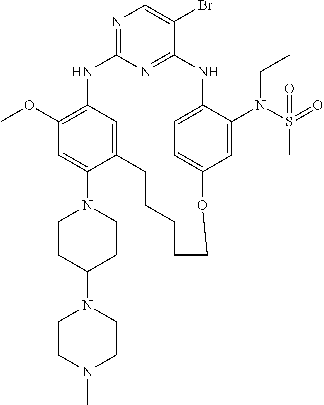 [SMILES: CCN(c1cc2ccc1Nc1nc(ncc1Br)Nc1cc(c(N3CCC(N4CCN(C)CC4)CC3)cc1OC)CCCCCO2)S(C)(=O)=O] | N-(3⁵-bromo-1⁶-methoxy-1⁴-(4-(4- methylpiperazin-1-yl)piperidin-1-yl)-6-oxa- 2,4-diaza-3(2,4)-pyrimidina-1(1,3),5(1,4)- dibenzenacycloundecaphane-5²-yl)-N- ethylmethanesulfonamide |
| A31 | 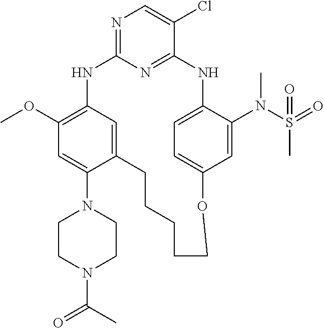 [SMILES: COc1cc(N2CCN(C(C)=O)CC2)c2cc1Nc1ncc(Cl)c(n1)Nc1ccc(cc1N(C)S(C)(=O)=O)OCCCCC2] | N-(1⁴-(4-acetylpiperazin-1-yl)-3⁵- chloro-1⁶-methoxy-6-oxa-2,4-diaza-3(2,4)- pyrimidina-1(1,3),5(1,4)- dibenzenacycloundecaphane-5²-yl)-N- methylmethanesulfonamide |
| A32 | 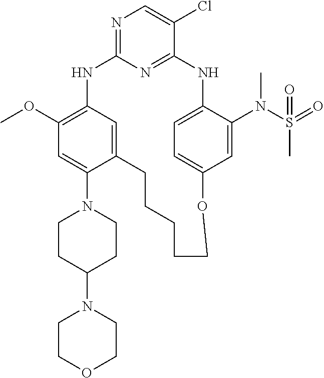 [SMILES: COc1cc(N2CCC(N3CCOCC3)CC2)c2cc1Nc1ncc(Cl)c(n1)Nc1ccc(cc1N(C)S(C)(=O)=O)OCCCCC2] | N-(3⁵-chloro-1⁶-methoxy-1⁴-(4- morpholinopiperidin-1-yl)-6-oxa-2,4-diaza- 3(2,4)-pyrimidina-1(1,3),5(1,4)- dibenzenacycloundecaphane-5²-yl)-N- methylmethanesulfonamide |
| A33 | 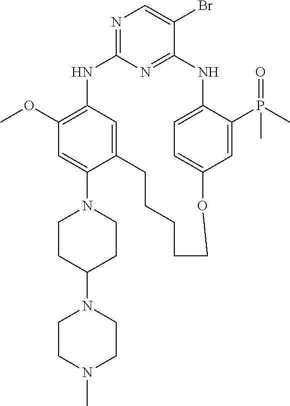 | (3⁵-bromo-1⁶-methoxy-1⁴-(4-(4- methylpiperazin-1-yl)piperidin-1-yl)-6-oxa- 2,4-diaza-3(2,4)-pyrimidina-1(1,3),5(1,4)- dibenzenacycloundecaphane-5²- yl)dimethylphosphine oxide |
| A34 | 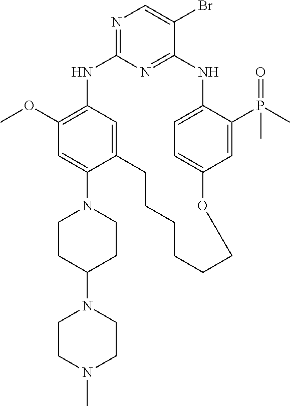 | (3⁵-bromo-1⁶-methoxy-1⁴-(4-(4- methylpiperazin-1-yl)piperidin-1-yl)-6-oxa- 2,4-diaza-3(2,4)-pyrimidina-1(1,3),5(1,4)- dibenzenacyclododecaphane-5²- yl)dimethylphosphine oxide |
| A35 | 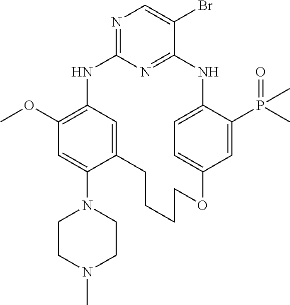 | (3⁵-bromo-1⁶-methoxy-1⁴-(4- methylpiperazin-1-yl)-6-oxa-2,4-diaza-3(2,4)- pyrimidina-1(1,3),5(1,4)- dibenzenacyclodecaphane-5²- yl)dimethylphosphine oxide |
| A36 | 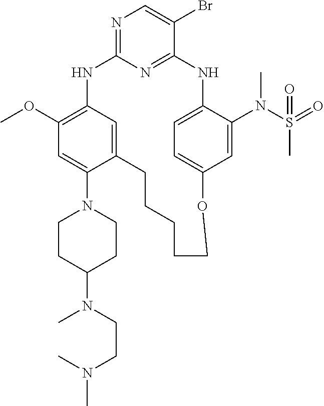 [SMILES: COc1cc(N2CCC(N(C)CCN(C)C)CC2)c2cc1Nc1ncc(Br)c(n1)Nc1ccc(cc1N(C)S(C)(=O)=O)OCCCCC2] | N-(3⁵-bromo-1⁴-(4-((2- (dimethylamino)ethyl)(methyl)amino) piperidin-1-yl)-1⁶-methoxy-6-oxa-2,4- diaza-3(2,4)-pyrimidina-1(1,3),5(1,4)- dibenzenacycloundecaphane-5²-yl)-N- methylmethanesulfonamide |
| A37 | 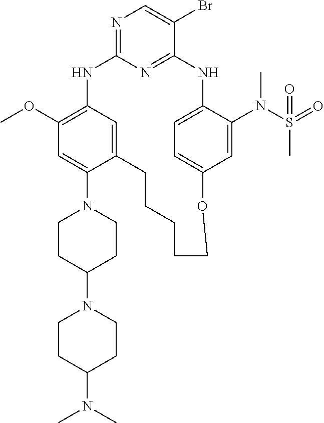 [SMILES: COc1cc(N2CCC(N3CCC(N(C)C)CC3)CC2)c2cc1Nc1ncc(Br)c(n1)Nc1ccc(cc1N(C)S(C)(=O)=O)OCCCCC2] | N-(3⁵-bromo-1⁴-(4-(dimethylamino)- [1,4′-bipiperidin]-1′-yl)-1⁶-methoxy-6-oxa-2,4- diaza-3(2,4)-pyrimidina-1(1,3),5(1,4)- dibenzenacycloundecaphane-5²-yl)-N- methylmethanesulfonamide |
| A38 | 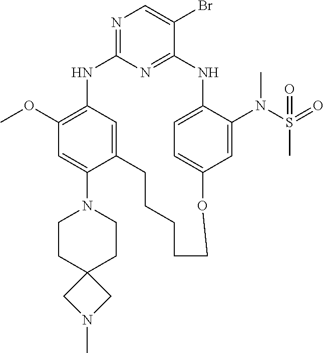 [SMILES: COc1cc(N2CCC3(CC2)CN(C)C3)c2cc1Nc1ncc(Br)c(n1)Nc1ccc(cc1N(C)S(C)(=O)=O)OCCCCC2] | N-(3⁵-bromo-1⁶-methoxy-1⁴-(2- methyl-2,7-diazaspiro[3.5]nonan-7-yl)-6-oxa- 2,4-diaza-3(2,4)-pyrimidina-1(1,3),5(1,4)- dibenzenacycloundecaphane-5²-yl)-N- methylmethanesulfonamide |
| A39 | 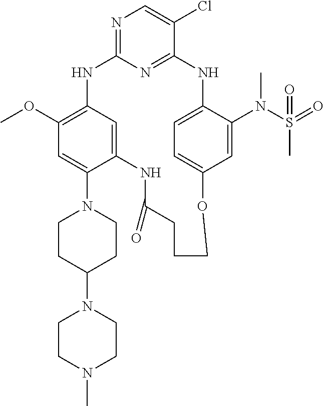 [SMILES: COc1cc(N2CCC(N3CCN(C)CC3)CC2)c2cc1Nc1ncc(Cl)c(n1)Nc1ccc(cc1N(C)S(C)(=O)=O)OCCCC(=O)N2] | N-(3⁵-chloro-1⁶-methoxy-1⁴-(4-(4- methylpiperazin-1-yl)piperidin-1-yl)-10-oxo- 6-oxa-2,4,11-triaza-3(2,4)-pyrimidina- 1(1,3),5(1,4)-dibenzenacycloundecaphane-5²- yl)-N-methylmethanesulfonamide |
| A40 | 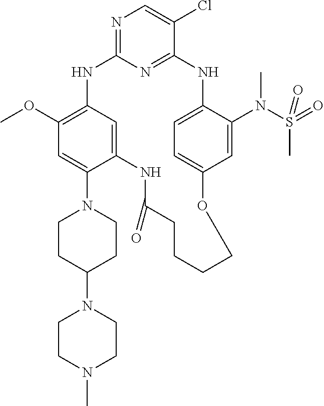 [SMILES: COc1cc(N2CCC(N3CCN(C)CC3)CC2)c2cc1Nc1ncc(Cl)c(n1)Nc1ccc(cc1N(C)S(C)(=O)=O)OCCCCC(=O)N2] | N-(3⁵-chloro-1⁶-methoxy-1⁴-(4-(4- methylpiperazin-1-yl)piperidin-1-yl)-11-oxo- 6-oxa-2,4,12-triaza-3(2,4)-pyrimidina- 1(1,3),5(1,4)-dibenzenacyclododecaphane-5²- yl)-N-methylmethanesulfonamide |
| A41 | 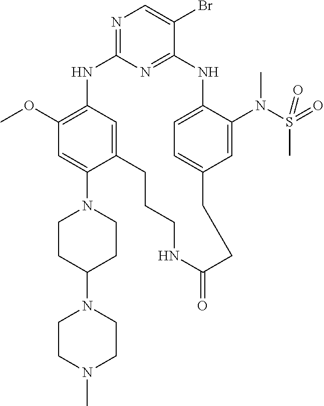 [SMILES: COc1cc(N2CCC(N3CCN(C)CC3)CC2)c2cc1Nc1ncc(Br)c(n1)Nc1ccc(cc1N(C)S(C)(=O)=O)CCC(=O)NCCC2] | N-(3⁵-chloro-1⁶-methoxy-1⁴-(4-(4- methylpiperazin-1-yl)piperidin-1-yl)-8-oxo- 2,4,9-triaza-3(2,4)-pyrimidina-1(1,3),5(1,4)- dibenzenacyclododecaphane-5²-yl)-N- methylmethanesulfonamide |
| A42 | 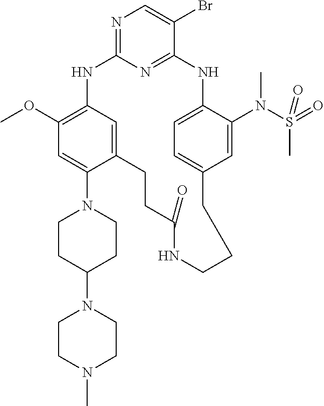 [SMILES: COc1cc(N2CCC(N3CCN(C)CC3)CC2)c2cc1Nc1ncc(Br)c(n1)Nc1ccc(cc1N(C)S(C)(=O)=O)CCCNC(=O)CC2] | N-(3⁵-chloro-1⁶-methoxy-1⁴-(4-(4- methylpiperazin-1-yl)piperidin-1-yl)-10-oxo- 2,4,9-triaza-3(2,4)-pyrimidina-1(1,3),5(1,4)- dibenzenacyclododecaphane-5²-yl)-N- methylmethanesulfonamide |

Some embodiments include an optionally substituted compound or core structure from Table 1. A core structure is a compound of Table 1 with the substituents, such as Me, —OCH₃, F, Cl, Br, and —N(Me)S(O)₂Me groups removed.

Some embodiments include a pharmaceutical composition comprising a subject compound described herein, such as a compound of Formula 1, 1a, 1b 1c, 1d, 1e, 1f, 1g, 1h or 2, for example optionally substituted 3⁵-chloro-1⁶-methoxy-1⁴-(4-methylpiperazin-1-yl)-5²-(methylsulfonyl)-6-oxa-2,4-diaza-3(2,4)-pyrimidina-1(1,3),5(1,4)-dibenzenacyclononaphane, optionally substituted N-(3⁵-chloro-1⁶-methoxy-1⁴-(4-methylpiperazin-1-yl)-6-oxa-2,4-diaza-3(2,4)-pyrimidina-1(1,3),5(1,4)-dibenzenacyclononaphane-5²-yl)-N-methylmethanesulfonamide, optionally substituted N-(3⁵-chloro-1⁶-methoxy-1⁴-(4-methylpiperazin-1-yl)-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-chloro-1⁶-methoxy-1⁴-(4-methylpiperazin-1-yl)-6-oxa-2,4-diaza-3(2,4)-pyrimidina-1(1,3),5(1,4)-dibenzenacyclodecaphane-5²-yl)-N-methylmethanesulfonamide, optionally substituted N-(3⁵-chloro-1⁴-((2-(dimethylamino)ethyl)(methyl)amino)-1⁶-methoxy-6-oxa-2,4-diaza-3(2,4)-pyrimidina-1(1,3),5(1,4)-dibenzenacycloundecaphane-5²-yl)-N-methylmethanesulfonamide, optionally substituted 3⁵-chloro-1⁶-methoxy-1⁴-(4-methylpiperazin-1-yl)-5²-(methylsulfonyl)-6-oxa-2,4-diaza-3(2,4)-pyrimidina-1(1,3),5(1,4)-dibenzenacyclodecaphane, optionally substituted N-(3⁵-chloro-1⁴-((2-(dimethylamino)ethyl)(methyl)amino)-1⁶-methoxy-6-oxa-2,4-diaza-3(2,4)-pyrimidina-1(1,3),5(1,4)-dibenzenacyclononaphane-5²-yl)-N-methylmethanesulfonamide, optionally substituted N-(3⁵-chloro-1⁶-methoxy-1⁴-(4-methylpiperazin-1-yl)-6-oxa-2,4-diaza-3(2,4)-pyrimidina-1(1,3),5(1,4)-dibenzenacycloundecaphane-5²-yl)-N-methylethanesulfonamide, optionally substituted N-(3⁵-chloro-1⁶-methoxy-1⁴-(4-(4-methylpiperazin-1-yl)piperidin-1-yl)-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-chloro-1⁶-methoxy-1⁴-(4-methylpiperazin-1-yl)-6-oxa-2,4-diaza-3(2,4)-pyrimidina-1(1,3),5(1,4)-dibenzenacycloundecaphane-5²-yl)-N-ethylmethanesulfonamide, optionally substituted N-(3⁵-fluoro-1⁶-methoxy-1⁴-(4-methyl piperazin-1-yl)-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-bromo-1⁶-methoxy-1⁴-(4-methylpiperazin-1-yl)-6-oxa-2,4-diaza-3(2,4)-pyrimidina-1(1,3),5(1,4)-dibenzenacycloundecaphane-5²-yl)-N-methylmethanesulfonamide, optionally substituted N-(1⁶-methoxy-1⁴-(4-methylpiperazin-1-yl)-3⁵-(trifluoromethyl)-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-chloro-1⁶-methoxy-1⁴-(4-methylpiperazin-1-yl)-6-oxa-2,4-diaza-3(2,4)-pyrimidina-1(1,3),5(1,4)-dibenzenacyclododecaphane-5²-yl)-N-methylmethanesulfonamide, optionally substituted N-(3⁵-chloro-1⁶-methoxy-14-(4-(4-methylpiperazin-1-yl)piperidin-1-yl)-6-oxa-2,4-diaza-3(2,4)-pyrimidina-1(1,3),5(1,4)-dibenzenacyclododecaphane-5²-yl)-N-methylmethanesulfonamide, optionally substituted N-(3⁵-chloro-1⁶-methoxy-1⁴-(4-methylpiperazin-1-yl)-6-oxa-2,4-diaza-3(2,4)-pyrimidina-1(1,3),5(1,4)-dibenzenacycloundecaphane-5²-yl)methanesulfonamide, optionally substituted N-(3⁵-chloro-1⁶-methoxy-1⁴-(4-methylpiperazin-1-yl)-6-oxa-2,4-diaza-3(2,4)-pyrimidina-1(1,3),5(1,4)-dibenzenacycloundecaphane-5²-yl)-N-methylpropane-2-sulfonamide, optionally substituted (3⁵-chloro-1⁶-methoxy-1⁴-(4-methylpiperazin-1-yl)-6-oxa-2,4-diaza-3(2,4)-pyrimidina-1(1,3),5(1,4)-dibenzenacycloundecaphane-5²-yl)dimethylphosphine oxide, optionally substituted 3⁵-chloro-1⁶-methoxy-1⁴-(4-(4-methylpiperazin-1-yl)piperidin-1-yl)-5²-(methylsulfonyl)-6-oxa-2,4-diaza-3(2,4)-pyrimidina-1(1,3),5(1,4)-dibenzenacyclododecaphane, optionally substituted N-(3⁵-chloro-1⁶-methoxy-1⁴-(4-(1-methylpiperidin-4-yl)piperazin-1-yl)-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-chloro-1⁶-ethoxy-1⁴-(4-methylpiperazin-1-yl)-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-chloro-1⁶-isopropoxy-1⁴-(4-methylpiperazin-1-yl)-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-chloro-1⁶-methoxy-1⁴-(4-methylpiperazin-1-yl)-6-oxa-2,4-diaza-3(2,4)-pyrimidina-1(1,3),5(1,4)-dibenzenacyclododecaphane-5²-yl)-N-ethylmethanesulfonamide, optionally substituted N-(3⁵-chloro-1⁴-(4-(dimethylamino)piperidin-1-yl)-1⁶-methoxy-6-oxa-2,4-diaza-3(2,4)-pyrimidina-1(1,3),5(1,4)-dibenzenacycloundecaphane-5²-yl)-N-methylmethanesulfonamide, optionally substituted 3⁵-chloro-1⁶-methoxy-N,N-dimethyl-1⁴-(4-methylpiperazin-1-yl)-6-oxa-2,4-diaza-3(2,4)-pyrimidina-1(1,3),5(1,4)-dibenzenacyclododecaphane-5²-carboxamide, optionally substituted 3⁵-chloro-5²-(isopropylsulfonyl)-16-methoxy-1⁴-(4-(4-methylpiperazin-1-yl)piperidin-1-yl)-6-oxa-2,4-diaza-3(2,4)-pyrimidina-1(1,3),5(1,4)-dibenzenacycloundecaphane, optionally substituted N-(3⁵-chloro-1⁴-(3-(dimethylamino)pyrrolidin-1-yl)-1⁶-methoxy-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-chloro-1⁶-methoxy-1⁴-((cis-3,6)-5-methylhexahydropyrrolo[3,4-c]pyrrol-2(1H)-yl)-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-bromo-1⁶-methoxy-1⁴-(4-(4-methylpiperazin-1-yl)piperidin-1-yl)-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-bromo-1⁶-methoxy-1⁴-(4-(4-methylpiperazin-1-yl)piperidin-1-yl)-6-oxa-2,4-diaza-3(2,4)-pyrimidina-1(1,3),5(1,4)-dibenzenacycloundecaphane-5²-yl)-N-ethylmethanesulfonamide, optionally substituted N-(1⁴-(4-acetylpiperazin-1-yl)-3⁵-chloro-1⁶-methoxy-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-chloro-1⁶-methoxy-1⁴-(4-morpholinopiperidin-1-yl)-6-oxa-2,4-diaza-3(2,4)-pyrimidina-1(1,3),5(1,4)-dibenzenacycloundecaphane-5²-yl)-N-methylmethanesulfonamide, optionally substituted (3⁵-bromo-1⁶-methoxy-1⁴-(4-(4-methylpiperazin-1-yl)piperidin-1-yl)-6-oxa-2,4-diaza-3(2,4)-pyrimidina-1(1,3),5(1,4)-dibenzenacycloundecaphane-5²-yl)dimethylphosphine oxide, optionally substituted (3⁵-bromo-1⁶-methoxy-1⁴-(4-(4-methylpiperazin-1-yl)piperidin-1-yl)-6-oxa-2,4-diaza-3(2,4)-pyrimidina-1(1,3),5(1,4)-dibenzenacyclododecaphane-5²-yl)dimethylphosphine oxide, optionally substituted (3⁵-bromo-1⁶-methoxy-1⁴-(4-methylpiperazin-1-yl)-6-oxa-2,4-diaza-3(2,4)-pyrimidina-1(1,3),5(1,4)-dibenzenacyclodecaphane-5²-yl)dimethylphosphine oxide, optionally substituted N-(3⁵-bromo-1⁴-(4-((2-(dimethylamino)ethyl)(methyl)amino)piperidin-1-yl)-1⁶-methoxy-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-bromo-1⁴-(4-(dimethylamino)-[1,4′-bipiperidin]-1′-yl)-1⁶-methoxy-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-bromo-1⁶-methoxy-1⁴-(2-methyl-2,7-diazaspiro[3.5]nonan-7-yl)-6-oxa-2,4-diaza-3(2,4)-pyrimidina-1(1,3),5(1,4)-dibenzenacycloundecaphane-5²-yl)-N-methylmethanesulfonamide, optionally substituted N-(3⁵-chloro-1⁶-methoxy-1⁴-(4-(4-methylpiperazin-1-yl)piperidin-1-yl)-10-oxo-6-oxa-2,4,11-triaza-3(2,4)-pyrimidina-1(1,3),5(1,4)-dibenzenacycloundecaphane-5²-yl)-N-methylmethanesulfonamide, optionally substituted N-(3⁵-chloro-1⁶-methoxy-1⁴-(4-(4-methylpiperazin-1-yl)piperidin-1-yl)-11-oxo-6-oxa-2,4,12-triaza-3(2,4)-pyrimidina-1(1,3),5(1,4)-dibenzenacyclododecaphane-5²-yl)-N-methylmethanesulfonamide, or optionally substituted N-(3⁵-chloro-1⁶-methoxy-1⁴-(4-(4-methylpiperazin-1-yl)piperidin-1-yl)-8-oxo-2,4,9-triaza-3(2,4)-pyrimidina-1(1,3),5(1,4)-dibenzenacyclododecaphane-5²-yl)-N-methylmethanesulfonamide, or optionally substituted N-(3⁵-chloro-1⁶-methoxy-1⁴-(4-(4-methylpiperazin-1-yl)piperidin-1-yl)-10-oxo-2,4,9-triaza-3(2,4)-pyrimidina-1(1,3),5(1,4)-dibenzenacyclododecaphane-5²-yl)-N-methylmethanesulfonamide, or a pharmaceutically acceptable salt thereof, and at least one pharmaceutically acceptable carrier.

An example, not as an attempt to limit the scope of the disclosure, of a useful composition for a dosage form containing about 10-1000 mg of compound A29 is shown in Table 2 below:

TABLE 2
Example of dosage form of compound A29

| Component | Amount (wt/wt) |
|---|---|
| Compound A29 | 30-70% |
| lubricant | 1-10% |
| diluent | 20-70% |
| disintegrant | 1-10% |

In some embodiments, a dosage form may comprise about 10-2000 mg of a subject compound described herein. In some embodiments, a dosage form may contain about 10-20 mg, about 20-30 mg, about 30-40 mg, about 40-50 mg, about 50-60 mg, about 60-70 mg, about 70-80 mg, about 80-90 mg, about 90-100 mg, about 100-150 mg, about 150-200 mg, about 200-250 mg, about 250-300 mg, about 300-350 mg, about 350-400 mg, about 400-450 mg, about 450-500 mg, about 500-600 mg, about 600-700 mg, about 700-800 mg, about 800-900 mg, about 900-1000 mg, about 1000-1500 mg, about 1500-2000 mg, about 10-50 mg, about 50-100 mg, about 100-200 mg, about 200-300 mg, about 300-400 mg, about 400-500 mg, about 10-2000 mg, about 10-1000 mg, about 10-500 mg, or any amount in a range bounded by any of the above values of a subject compound, such as a compound of Formula 1, 1a, 1b, 1c, 1d, 1e, 1f, 1g, 1h, or 2. The term “about 10-500 mg” described herein means about 10 mg to about 500 mg, and so on.

In some embodiments, a daily dose of a subject compound described herein may be in a range of about 1-100 mg/kg. In some embodiments, a daily dose may be about 1-5 mg/kg, about 5-10 mg/kg, about 10-15 mg/kg, about 15-20 mg/kg, about 20-25 mg/kg, about 25-30 mg/kg, about 30-35 mg/kg, about 35-40 mg/kg, about 40-45 mg/kg, about 45-50 mg/kg, about 50-55 mg/kg, about 55-60 mg/kg, about 60-65 mg/kg, about 65-70 mg/kg, about 70-75 mg/kg, about 75-80 mg/kg, about 80-85 mg/kg, about 85-90 mg/kg, about 60-95 mg/kg, about 95-100 mg/kg, about 1-60 mg/kg, about 1-50 mg/kg, about 1-40 mg/kg, about 1-30 mg/kg, about 1-10 mg/kg, about 10-20 mg/kg, about 20-30 mg/kg, about 30-40 mg/kg, about 40-50 mg/kg, about 50-60 mg/kg, about 60-70 mg/kg, about 70-80 mg/kg, about 80-90 mg/kg, about 90-100 mg/kg, about 1 mg/kg, about 5 mg/kg, about 10 mg/kg, about 20 mg/kg, about 30 mg/kg, about 40 mg/kg, about 50 mg/kg, about 60 mg/kg, or any amount in a range bounded by any of the above values of a subject compound, such as a compound of Formula 1, 1a, 1b, 1c, 1d, 1e, 1f, 1g, 1h, or 2. The term “about 1-60 mg/kg” described herein means about 1 mg/kg to about 60 mg/kg, and so on.

In some embodiments, the dosage form may comprise about 10-95% by weight of a subject compound described herein as compared to the total weight of the dosage form. In some embodiments, the dosage form may contain about 10-15%, about 15-20%, about 20-25%, about 25-30%, about 30-35%, about 35-40%, about 40-45%, about 45-50%, about 50-55%, about 55-60%, about 60-65%, about 65-70%, about 70-75%, about 75-80%, about 80-85%, about 85-90%, about 90-95%, about 10-20%, about 20-30%, about 30-40%, about 40-50%, about 50-60%, about 60-70%, about 70-80%, about 80-90%, about 10-30%, about 30-50%, about 50-70%, about 70-90%, or about 30-70%, about 30%, about 40%, about 50%, about 55%, about 60%, about 70% by weight of the total weight of the dosage form of a subject compound, such as a compound of Formula 1, 1a, 1b, 1c, 1d, 1e, 1f, or 2. The term “about 30-70%” described herein means about 30% to about 70%, and so on.

In some embodiments, a pharmaceutical composition comprising a subject compound, such as a compound of Formula 1, 1a, 1b, 1c, 1d, 1e, 1f, 1g, 1h, or 2 may be adapted for oral, or parental, such as intravenous, intramuscular, topical, intraperitoneal, nasal, buccal, sublingual, or subcutaneous administration, or for administration via respiratory tract in the form of, for example, an aerosol or an air-suspended fine powder. The dosage of a subject compound, such as a compound of Formula 1,1a, 1b, 1c, 1d, 1e, 1f, 1g, 1h, or 2 may vary depending on the route of administration, body weight, age, the type and condition of the disease being treated. A subject pharmaceutical composition provided herein may optionally comprise two or more compounds of the Formula 1, 1a, 1b, 1c, 1d, 1e, 1f, 1g, 1h, or 2 without an additional therapeutic agent, or may comprise an additional therapeutic agent (i.e., a therapeutic agent other than a compound provided herein). For example, the subject compounds of the disclosure can be administered simultaneously, sequentially, separately, or in a single dosage form in combination with at least one other therapeutic agent. Therapeutic agents suitable for combination include, but are not limited to antibiotics, antiemetic agents, antidepressants, and antifungal agents, antiinflammatory agents, antiviral agents, and anticancer agents that are known in the art. The pharmaceutical composition may be used for the treatment of a disease, a condition, or a disorder which responds to the inhibition of epidermal growth factor receptor (EGFR), anaplastic lymphoma kinase (ALK) activity, or a combination thereof, such as cancer in mammals. The term “mammal” herein means a human or an animal. In some embodiments, the mammal has cancer. Such combination may offer significant advantages, including synergistic therapeutic effects. The subject pharmaceutical composition may be used for the treatment of cancer and other diseases or disorders, which respond to the inhibition of epidermal growth factor receptor (EGFR), anaplastic lymphoma kinase (ALK) activity, or a combination thereof in mammal. The term “mammal” herein means a human or an animal. In some embodiments, the mammal has cancer.

The subject pharmaceutical composition described herein can be prepared by combining a subject compound, such as a compound of Formula 1, 1a, 1b, 1c, 1d, 1e, 1f, 1g, 1h, or 2 with at least one pharmaceutical acceptable inert ingredient, such as a carrier, excipient, filler, lubricant, flavoring agent, buffer, etc., selected on the basis of the chosen route of administration and standard pharmaceutical practice as described, for example, in Remington's Pharmaceutical Sciences, 2005, the disclosure of which is hereby incorporated herein by reference, in its entirety. The relative proportions of active ingredient and carrier may be determined, for example, by the solubility and chemical nature of the compounds, chosen route of administration and standard pharmaceutical practice.

Some embodiments include a method of treating a disease, a disorder, or a condition, which responds to the inhibition of epidermal growth factor receptor (EGFR), anaplastic lymphoma kinase (ALK) activity, or a combination thereof, comprising administering a therapeutically effective amount of a subject compound, such as a compound of Formula 1, 1a, 1b, 1c, 1d, 1e, 1f, 1g, 1h, or 2, or any compound described herein, or a pharmaceutically acceptable salt thereof (“subject compound”), or a pharmaceutical composition comprising a subject compound to a mammal in need thereof. The term a “therapeutically effective amount” herein refers to an amount of a subject compound, or a pharmaceutical composition containing a subject compound, sufficient to be effective in inhibiting epidermal growth factor receptor (EGFR), anaplastic lymphoma kinase (ALK) activity, or a combination thereof, and thus providing a benefit in the treatment of a disease, a disorder, or a condition, such as cancer, in mammals, such as to delay or minimize symptoms associated with cancer, or to ameliorate a disease, a disorder or a condition, or a cause thereof, or to prevent the further development of a disease, a disorder or a condition, or reducing the severity of symptoms that are otherwise expected to develop without treatment.

Many of the subject compounds described herein are very potent and selective, with enzymatic IC₅₀ less than 10 nM, or less than 1 nM. Some of the compounds described herein could display superior anti-tumor activities in in vivo animal models. In some embodiments, administration of a subject compound described herein, such as A29, with a dose amount falls within the range of 1 mg/kg per day to 100 mg/kg per day could achieve the tumor regression or at least about 10%, at least about 20%, at least about 30%, at least about 40%, at least about 50%, at least about 60%, at least about 70%, at least about 80%, at least about 90%, at least about 95%, about 10-20%, about 20-30%, about 30-40%, about 40-50%, about 50-60%, about 60-70%, about 70-80%, about 80-90%, about 90-100%, about 10-30%, about 30-50%, about 50-70%, about 70-90%, about 90-100%, about 50-55%, about 55-60%, about 60-65%, about 65-70%, about 70-75%, about 75-80%, about 80-85%, about 85-90%, about 90-95%, about 95-100%, about 100% tumor growth inhibition in in vivo animal models. In some embodiments, administration of a subject compound described herein with a dose amount falls within the range of 1 mg/kg per day to 100 mg/kg per day could achieve the tumor regression or at least about 60% tumor growth inhibition in in vivo animal models. Such in vivo animal models include, but not limit to, Cell Transplant Xenograft (CTX) Model.

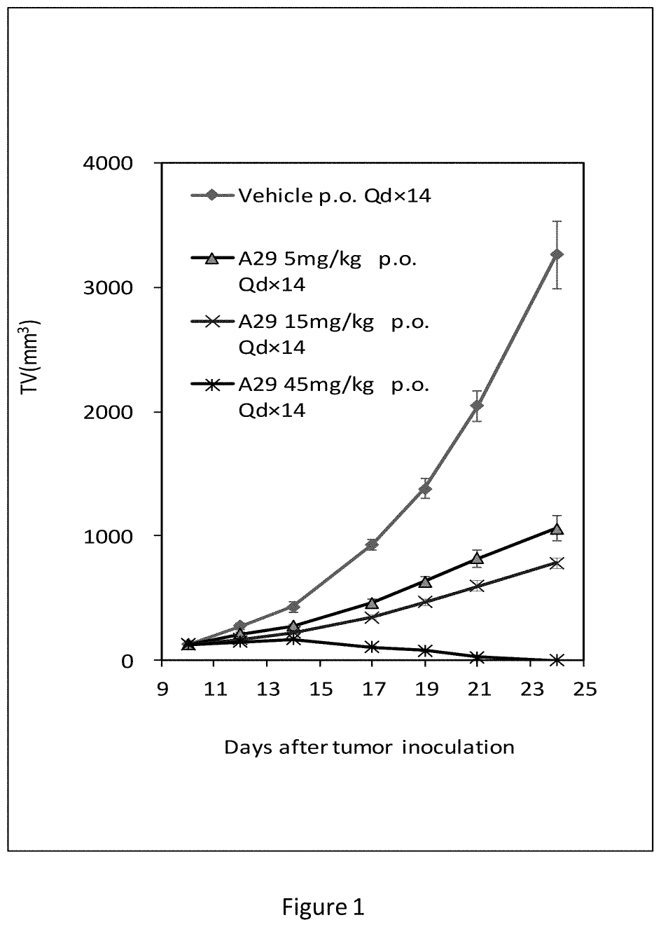

Therefore, the protein kinase inhibitors described herein, such as a compound of Formula 1, 1a, 1b, 1c, 1d, 1e, 1f, 1g, 1h, or 2, could be used in the treatment of cancer, as they can inhibit tumor growth significantly and with 100% inhibition at certain dose amount as shown in FIG. 1. For example, these subject compounds as protein kinase inhibitors described herein may be used to treat, ameliorate or prevent a disease, a disorder, or a condition which responds to inhibition of epidermal growth factor receptor (EGFR), anaplastic lymphoma kinase (ALK) activity, or a combination thereof. As a result of their inhibitory activity against the EGFR mutants (such as L858R, L858R/T790M, L858R/T790M/C797S, the ExonI9 deletion, the ExonI9 deletion/T790M, the ExonI9 deletion/T790M/C797S), and/or ALK mutant (such as EML4-ALK), the compounds of Formula 1, 1a, 1b, 1c, 1d, 1e, 1f, 1g, 1h, or 2, and pharmaceutically acceptable salts thereof, are expected to be useful in the treatment of diseases or medical conditions mediated alone or in part by EGFR mutant or ALK activity, for example cancer. The types of cancers which may be susceptible to treatment using these subject compounds or pharmaceutically acceptable salts thereof, include, but are not limited to, ovarian cancer, cervical cancer, colorectal cancer, breast cancer, pancreatic cancer, glioma, glioblastoma, melanoma, prostate cancer, leukaemia, lymphoma, non-Hodgkins lymphoma, gastric cancer, lung cancer, hepatocellular cancer, gastric cancer, gastrointestinal stromal tumour (GIST), thyroid cancer, bile duct cancer, endometrial cancer, renal cancer, anaplastic large cell lymphoma, acute myeloid leukaemia (AML), multiple myeloma, melanoma, and mesothelioma. The anti-cancer treatment described herein may be applied as a sole therapy or may involve a combination with conventional surgery or radiotherapy or chemotherapy or immunotherapy.

The pharmaceutical compositions comprising a subject compound described herein may be suitable for administration to mammals, such as humans, to inhibit kinase activity, and for the treatment of disease or disorders such as cancer, inflammatory disorders (e.g. rheumatoid arthritis, inflammatory bowel disease, asthma, chronic obstructive pulmonary disease (COPD)), osteoarthritis, dermatosis (e.g. Atopic dermatitis, psoriasis), vascular proliferative disorders (e.g. Atherosclerosis, restenosis), autoimmune disorders (e.g. multiple sclerosis, tissue and organ rejection)); and inflammation associated with infection (e.g. Immune responses), neurodegeneration disorders (e.g. Alzheimer's disease, Parkinson's disease, motor neuron disease, neuropathic pain, triplet repeat disorders, astrocytoma, and neurodegeneration as result of akcoholic liver disease), ischemic injury (e.g. Stroke), and cachexia (e.g. Accelerated muscle protein breakdown that accompanies various physiological and pathological states (e.g. Nerve injury, fasting, fever, acidosis, HIV infection, cancer affliction, and certain endocrinopathies)).

Some embodiments include a product kit comprising a subject pharmaceutical composition comprising a therapeutical amount of a subject compound described herein, optionally in the form of a dosage form, and a label or instruction describing how to administer the subject pharmaceutical composition to a mammal, such as a human being, for the treatment of a disease, a condition, or a disorder, such as cancer, which responds to the inhibition of epidermal growth factor receptor (EGFR), anaplastic lymphoma kinase (ALK) activity, or a combination thereof.

### Experimental Section

### Preparation of Compounds

The compounds of Formula 1, 1a, 1b, 1c, 1d, 1e, 1f, 1g, 1h, or 2 of the disclosure may be prepared using the methods as shown in the following reaction schemes and description thereof, as well as relevant published literature procedures that may be used.

### General Synthetic Methods:

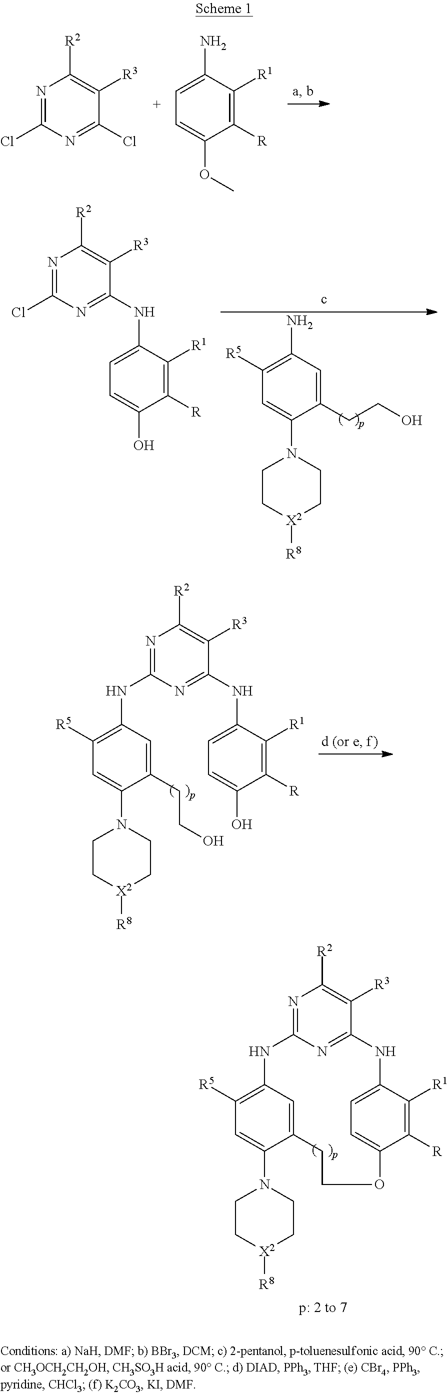

The intermediates shown in Scheme 1 may be prepared using the method shown in Scheme 2.

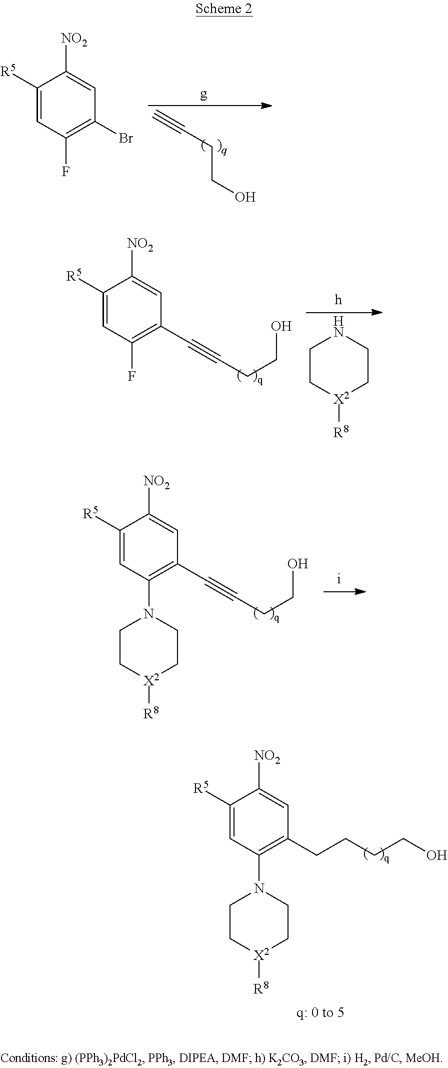

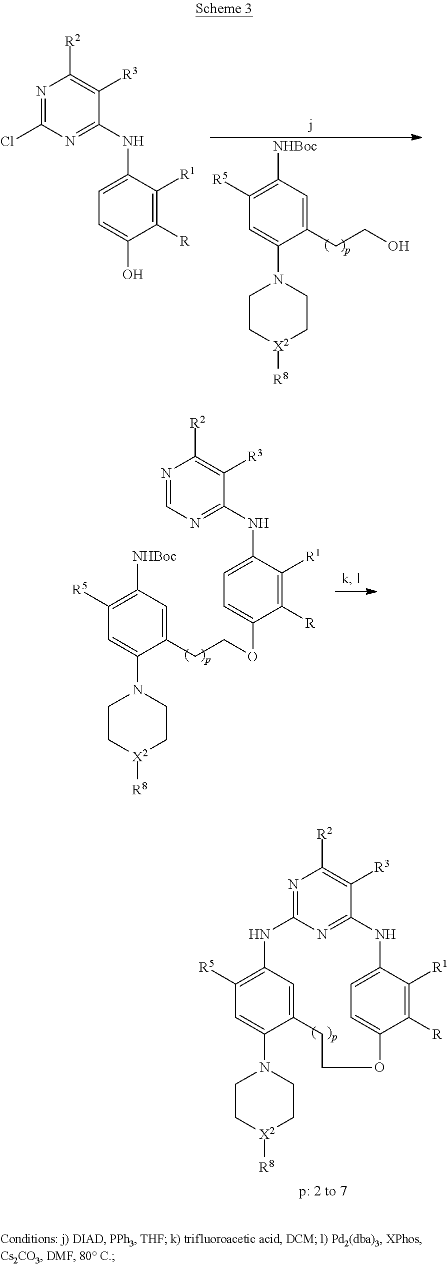

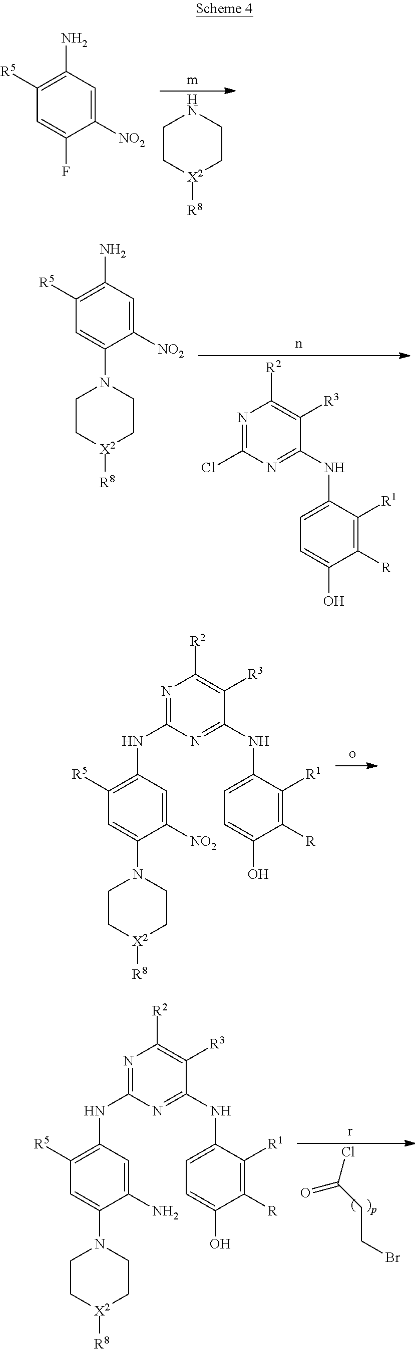

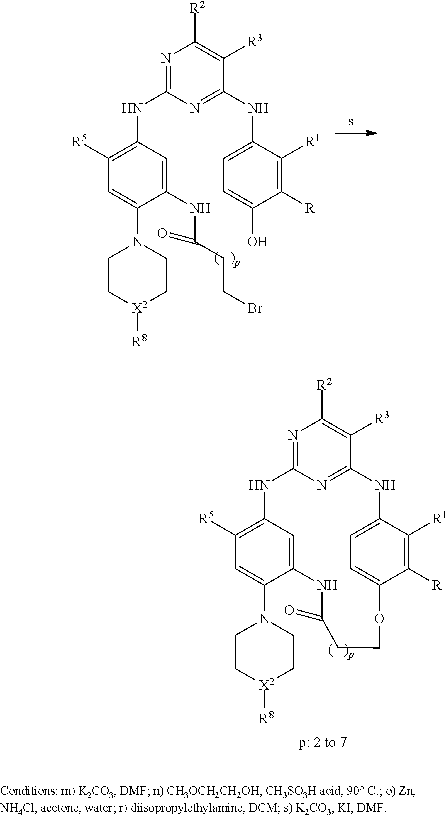

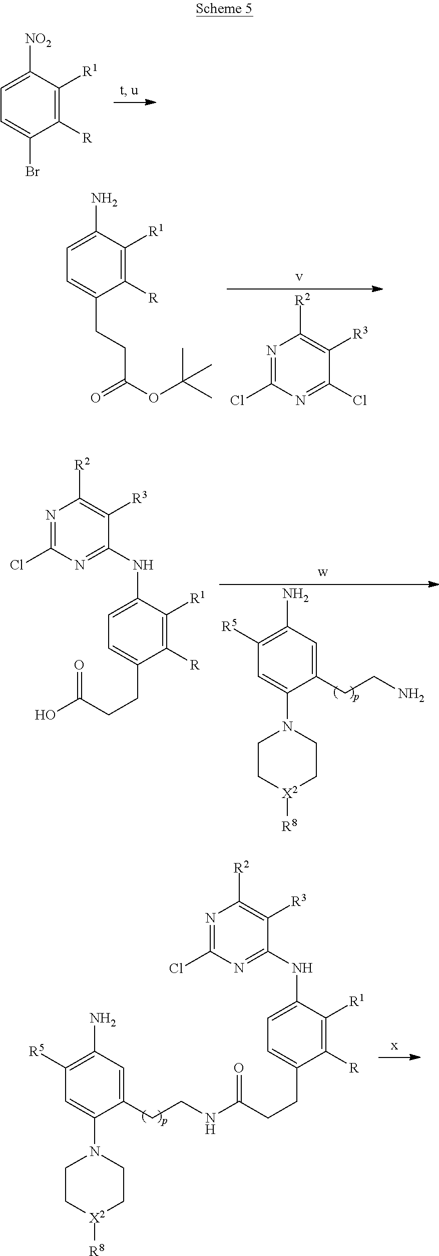

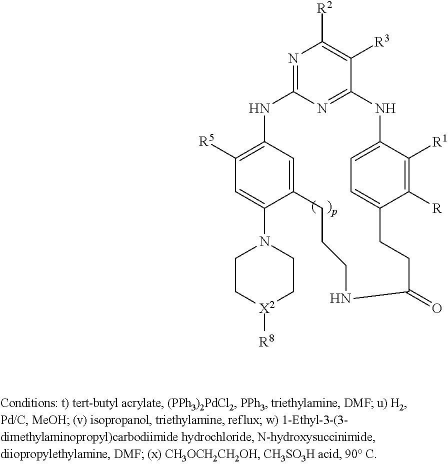

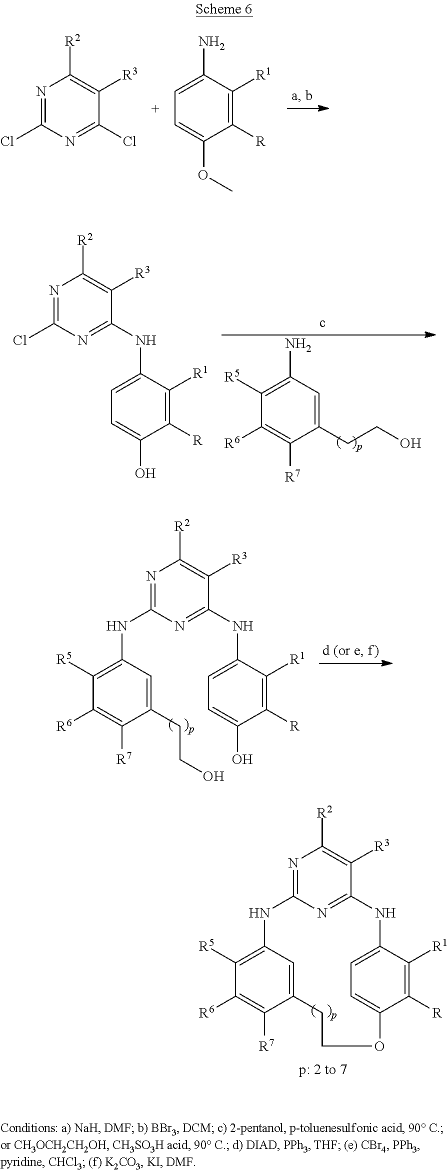

The intermediates shown in Scheme 6 may be prepared using the method shown in Scheme 7.

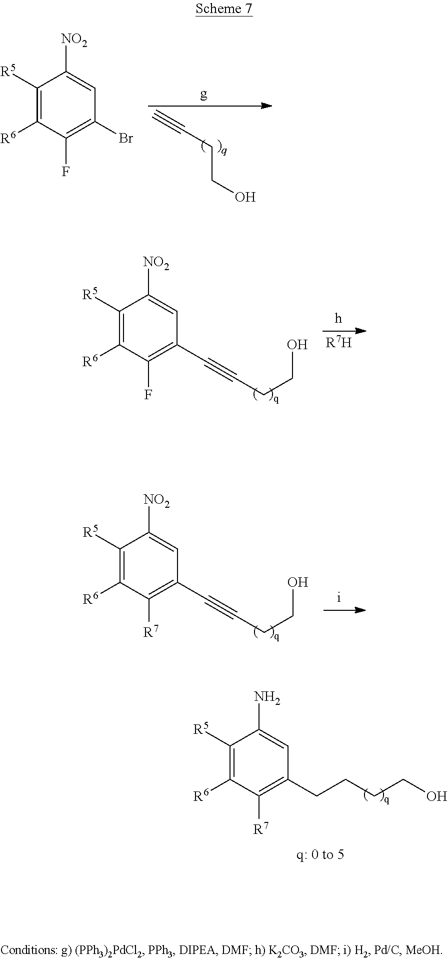

In another embodiment, synthesis of the compounds is shown in Scheme 3:

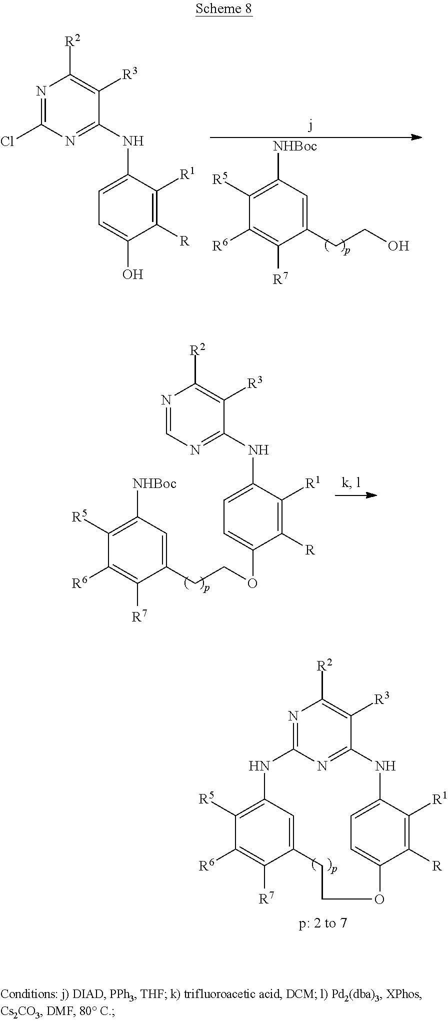

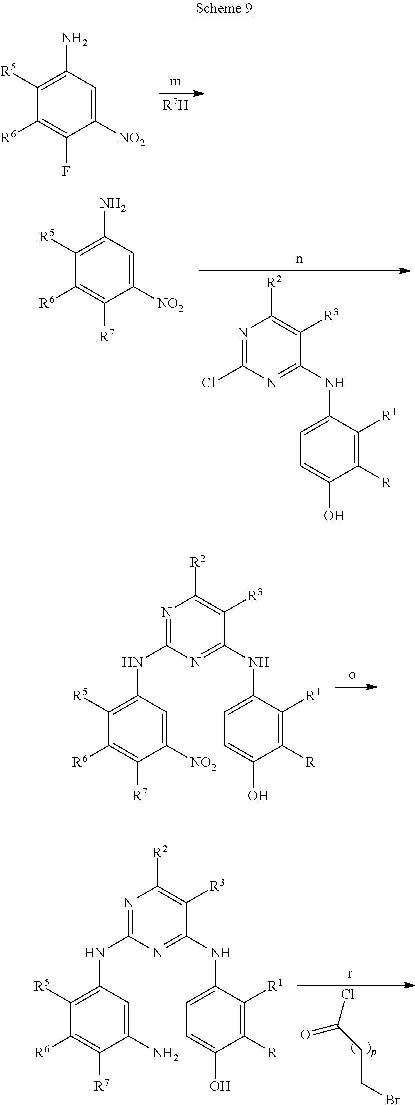

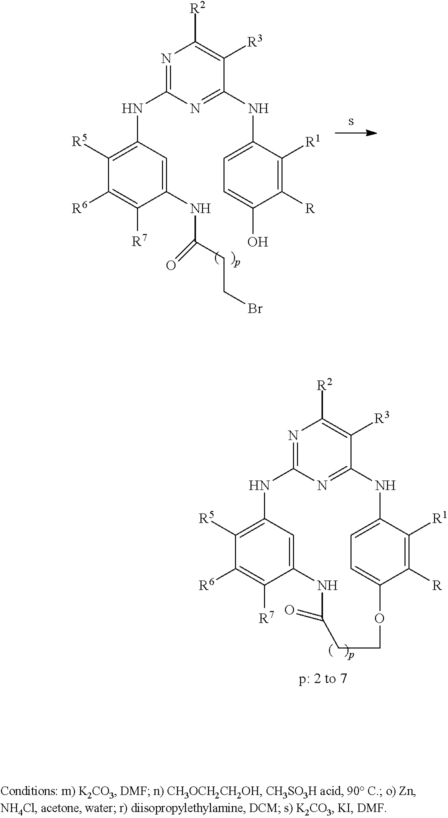

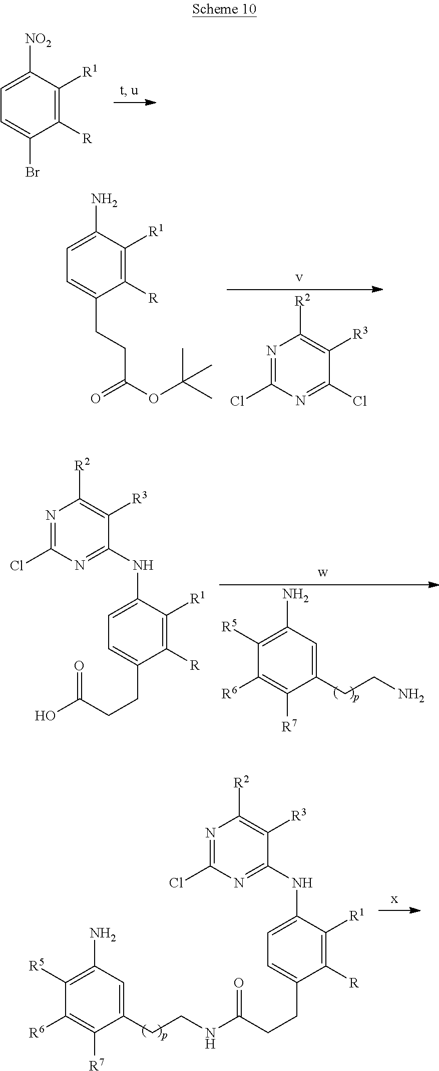

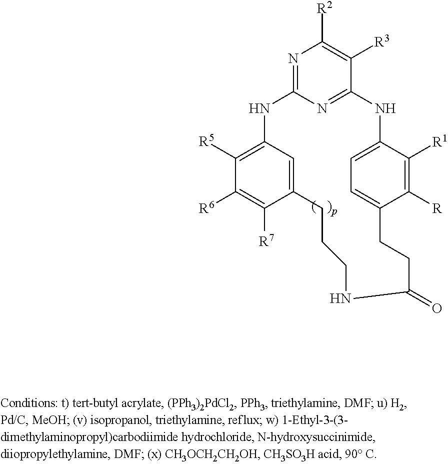

### EXAMPLES

The following examples show typical procedures for the synthesis of the compounds of the disclosure, but those skilled in the art will recognize that the following synthetic reactions and schemes may be modified by choice of suitable starting materials and reagents to prepare other compounds of Formula 1, 1a, 1b, 1c, 1d, 1e, 1f, 1g, or 2, and a variety of non-critical parameters can be changed or modified to prepare the same compounds.

### Example A1

### Synthesis of 3⁵-Chloro-1⁶-methoxy-1⁴-(4-methylpiperazin-1-yl)-5²-(methylsulfonyl)-6-oxa-2,4-diaza-3(2,4)-pyrimidina-1(1,3),5(1,4)-dibenzenacyclononaphane

### Step A: 4-methoxy-2-(methylsulfonyl)-1-nitrobenzene

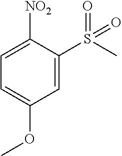

To a solution of sodium methoxide (540 mg, 10 mmol) in dry DMF (10 mL) at −40° C., a solution of 4-fluoro-2-(methylsulfonyl)-1-nitrobenzen (2.19g, 10 mmol) in dry DMF (10 mL) was added dropwise. The resulting mixture was stirred at −40° C. for about one hour, and monitored by LCMS. After the reaction finished, water (20 mL) was added to quench the reaction, and the product was extracted with chloroform/isopropanol (3:1) and purified by column chromatography on silica gel using hexane/ethyl acetate (70/30) (1.04 g, 45% yield). LCMS: m/z 232.2 [M+H]⁺.

### Step B: 4-methoxy-2-(methylsulfonyl)-1-nitrobenzene

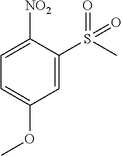

To a solution of 4-methoxy-2-(methylsulfonyl)-1-nitrobenzene (1.04g, 4.5 mmol) in methanol (50 mL) was added 10% Pd/C (50 mg). The reaction mixture was degassed, refilled with hydrogen gas, and stirred overnight at room temperature. After the reaction was completed, the mixture was filtered through Celite and washed with methanol. The combined filtrate was concentrated under vacuum to afford the crude product, which was used without further purification (0.9 g, 99% yield). LCMS m/z 202.2 (M+H)⁺.

### Step C: 2,5-dichloro-N-(4-methoxy-2-(methylsulfonyl)phenyl)pyrimidin-4-amine

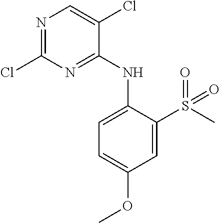 [SMILES: COc1ccc(Nc2nc(Cl)ncc2Cl)c(S(C)(=O)=O)c1]

To a solution of 4-methoxy-2-(methylsulfonyl)aniline (804 mg, 4 mmol) in dry DMF (10 ml) was added sodium hydride (60%, 320 mg, 8 mmol) at 0° C. and stirred for about 30 minutes. 2,4,5-trichloropyrimidine (1.1 g, 6 mmol) was added and the mixture was gradually warmed to room temperature and stirred overnight. After the reaction finished, water (20 mL) was added to quench the reaction, and the product was extracted with chloroform/isopropanol=3:1 and purified by column chromatography on silica gel using MeOH/dichloromethane (10/90) (850 mg, 61% yield). LCMS m/z 349.2 (M+H)⁺.

### Step D: 4-((2,5-dichloropyrimidin-4-yl)amino)-3-(methylsulfonyl)phenol

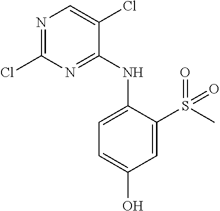 [SMILES: CS(=O)(=O)c1cc(O)ccc1Nc1nc(Cl)ncc1Cl]

To a solution of 2,5-dichloro-N-(4-methoxy-2-(methylsulfonyl)phenyl)pyrimidin-4-amine (696 mg, 2 mmol) in dichloromethane (5 ml) was added 2M BBr₃ in dichloromethane (5 ml, 5 mmol) at 0° C. The mixture was gradually warmed to room temperature and stirred overnight. Saturated aqueous NaHCO₃ solution was added to quench the reaction. The mixture was extracted with CHCl₃/isopropanol (3/1) mixture and the crude product was purified by column chromatography on silica gel using MeOH/dichloromethane (10/90) (410 mg, 61% yield). LCMS m/z 335.2 (M+H)⁺.

### Step E: 1-bromo-2 fluoro-4-methoxy-5-nitrobenzene

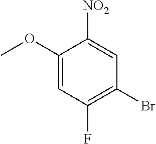

To a stirring solution of 1-bromo-2-fluoro-4-methoxybenzene (4.1 g, 20 mmol) in concentrated sulfonic acid (8 ml) was added concentrated nitric acid (60-70%, 2 ml, 30 mmol) at 0° C. The mixture was gradually warmed to room temperature and stirred for two hours. After the reaction finished, the mixture was poured into ice water, filtered to give crude product, which was purified by column chromatography on silica gel using hexane/ethyl acetate (80/20) (2 g, 40% yield). LCMS m/z 251.2 (M+H)⁺.

### Step F: 3-(2 fluoro-4-methoxy-5-nitrophenyl)prop-2-yn-1-ol

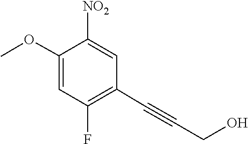 [SMILES: COc1cc(F)c(C#CCO)cc1[N+](=O)[O-]]

1-Bromo-2-fluoro-4-methoxy-5-nitrobenzene (1.0 g, 4 mmol), (PPh₃)₂PdCl₂ (280 mg, 0.4 mmol), CuI (167 mg, 0.8 mmol), triphenylphosphine (213 mg, 0.8 mmol) and 10 ml dry DMF were added into a 100 ml Schlenk tube. The mixture was degassed and refilled with argon. Then propargyl alcohol (448 mg, 8 mmol) and diisopropylethylamine (2.5 ml, 20 mmol) was added under argon. The mixture was stirred at 80° C. under argon overnight. The mixture was used for next step without purification. LCMS m/z 226.2 (M+H)⁺.

### Step G: 3-(2 fluoro-4-methoxy-5-nitrophenyl)prop-2-yn-1-ol

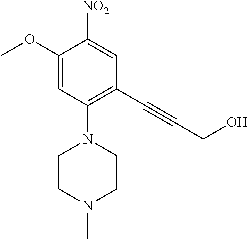 [SMILES: COc1cc(N2CCN(C)CC2)c(C#CCO)cc1[N+](=O)[O-]]

To the DMF solution of crude 3-(2-fluoro-4-methoxy-5-nitrophenyl)prop-2-yn-1-ol obtained from step F was added potassium carbonate (1.68 g, 12 mmol) and N-methylpiperazine (500 mg, 5 mmol). The mixture was heated to 80° C. for one hour, water (10 mL) was added to quench the reaction, and the product was extracted with chloroform/isopropanol=3:1 and purified by column chromatography on silica gel using MeOH/dichloromethane (10/90) (1 g, 82% yield for two steps). LCMS m/z 306.3 (M+H)⁺.

### Step H: 3-(5-amino-4-methoxy-2-(4-methylpiperazin-1-yl)phenyl)propan-1-ol

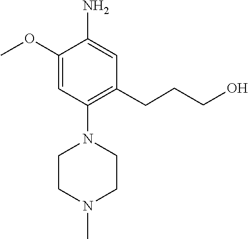 [SMILES: COc1cc(N2CCN(C)CC2)c(CCCO)cc1N]

To a solution of 3-(2-fluoro-4-methoxy-5-nitrophenyl)prop-2-yn-1-ol (1 g, 3.3 mmol) in methanol (50 ml) was added 10% Pd/C (50 mg). The reaction mixture was degassed, refilled with hydrogen gas, and stirred overnight at room temperature. The mixture was filtered through Celite and washed with methanol. The combined filtrate was collected and concentrated under vacuum to afford the crude product, which was purified by column chromatography on silica gel using MeOH/dichloromethane (10/90) (800 mg, 87% yield). LCMS m/z 280.4 (M+H)⁺.

### Step I: 4-((5-chloro-2-((5-(3-hydroxypropyl)-2-methoxy-4-(4-methylpiperazin-1-yl)phenyl)amino)pyrimidin-4-yl)amino)-3-(methylsulfonyl)phenol

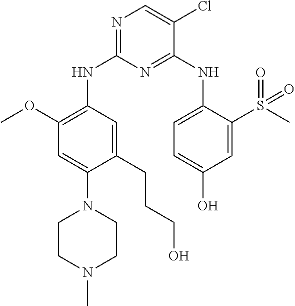

To a solution of 3-(2-fluoro-4-methoxy-5-nitrophenyl)prop-2-yn-1-ol (66.8 mg, 0.2 mmol) and 3-(5-amino-4-methoxy-2-(4-methylpiperazin-1-yl)phenyl)propan-1-ol (55.8 mg, 0.2 mmol) in 2-methoxyethanol (2 ml) was added methanesulfonic acid (58 mg, 0.6 mmol). The mixture was stirred at 90° C. overnight. Saturated aqueous NaHCO₃ solution (10 mL) was added to quench the reaction, and the product was extracted with chloroform/isopropanol=3:1 and purified by column chromatography on silica gel using MeOH/dichloromethane (10/90) (54 mg, 47% yield). LCMS m/z 578.1 (M+H)⁺.

### Step J: 3⁵-chloro-1⁶-methoxy-1⁴-(4-methylpiperazin-1-yl)-5²-(methylsulfonyl)-6-oxa-2,4-diaza-3(2,4)-pyrimidina-1(1,3),5(1,4)-dibenzenacyclononaphane

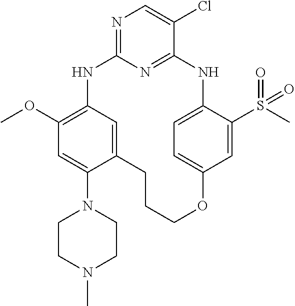

To a solution of 4-((5-chloro-2-((5-(3-hydroxypropyl)-2-methoxy-4-(4-methylpiperazin-1-yl)phenyl)amino)pyrimidin-4-yl)amino)-3-(methylsulfonyl)phenol (54 mg, 0.094 mmol) and triphenylphosphine (44 mg, 0.1 mmol) in anhydrous THE (5 ml) was added DIAD (20.2 mg, 0.1 mmol) at 0° C. The mixture was gradually warmed to room temperature and stirred overnight. The mixture was concentrated under vacuum and purified by column chromatography on silica gel using MeOH/dichloromethane (10/90) (3.1 mg, 6% yield). LCMS m/z 559.39 (M+H)⁺.

### Example A2

### N-(3⁵-chloro-1⁶-methoxy-1⁴-(4-methyl piperazin-1-yl)-6-oxa-2,4-diaza-3(2,4)-pyrimidina-1(1,3),5(1,4)-dibenzenacyclononaphane-5²-yl)-N-methylmethanesulfonamide

### Step A: N-(5-methoxy-2-nitrophenyl)-N-methylmethanesulfonamide

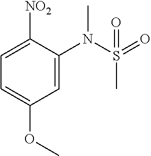 [SMILES: COc1ccc([N+](=O)[O-])c(N(C)S(C)(=O)=O)c1]

To a suspension of cesium carbonate (6.2 g, 20 mmol) in acetonitrile (100 ml) at room temperature was added N-methylmethanesulfonamide (1.64 g, 15 mmol) and 2-fluoro-4-methoxy-1-nitrobenzene (1.71 g, 10 mmol) dropwise over 15 minutes. The reaction was stirred overnight. Upon completion, the mixture was filtered and then concentrated. The residue was purified by column chromatography on silica gel using MeOH/dichloromethane (10/90) to afford the product (1.38 g, 53% yield). LCMS m/z 261.3 (M+H)⁺.

### Step B: N-(2-amino-5-methoxyphenyl)-N-methylmethanesulfonamide

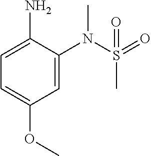

To a solution of N-(5-methoxy-2-nitrophenyl)-N-methylmethanesulfonamide (1.38 g, 5.3 mmol) in methanol (50 ml) was added 10% Pd/C (50 mg). The reaction mixture was degassed, refilled with hydrogen gas, and stirred to react overnight at room temperature. The mixture was filtered through Celite and washed with methanol. The combined filtrate was collected and concentrated under vacuum to afford the crude product, which was purified by column chromatography on silica gel using MeOH/dichloromethane (10/90) (1.15 g, 94% yield). LCMS m/z 231.3 (M+H)⁺.

### Step C: N-(2-((2,5-dichloropyrimidin-4-yl)amino)-5-methoxyphenyl)-N-methylmethanesulfonamide

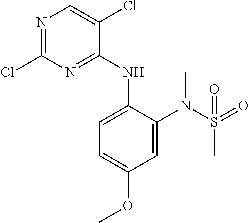 [SMILES: COc1ccc(Nc2nc(Cl)ncc2Cl)c(N(C)S(C)(=O)=O)c1]

To a solution of N-(2-amino-5-methoxyphenyl)-N-methylmethanesulfonamide (920 mg, 4 mmol) in dry DMF (20 ml) was added sodium hydride (60%, 320 mg, 8 mmol) at 0° C. and stir for about 30 minutes. 2,4,5-trichloropyrimidine (917 mg, 5 mmol) was added and the mixture was gradually warmed to room temperature and stirred overnight, Water (10 mL) was added to quench the reaction, the product was extracted with chloroform/isopropanol=3:1 and purified by column chromatography on silica gel using MeOH/dichloromethane (10/90) (816 mg, 54% yield). LCMS m/z 378.2 (M+H)⁺.

### Step D: N-(2-((2,5-dichloropyrimidin-4-yl)amino)-5-hydroxyphenyl)-N-methylmethanesulfonamide

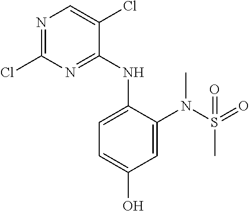 [SMILES: CN(c1cc(O)ccc1Nc1nc(Cl)ncc1Cl)S(C)(=O)=O]

To a solution of N-(2-((2,5-dichloropyrimidin-4-yl)amino)-5-methoxyphenyl)-N-methylmethanesulfonamide (754 mg, 2 mmol) in dichloromethane (5 ml) was added 1M BBr₃ in dichloromethane (5 ml, 5 mmol) at 0° C. The mixture was gradually warmed to room temperature and stirred overnight. Saturated aqueous NaHCO₃ solution was added to quench the reaction. The mixture was extracted with CHCl₃/isopropanol (3/1) mixture and the crude product was purified by column chromatography on silica gel using MeOH/dichloromethane (10/90) (500 mg, 69% yield). LCMS m/z 364.2 (M+H)⁺.

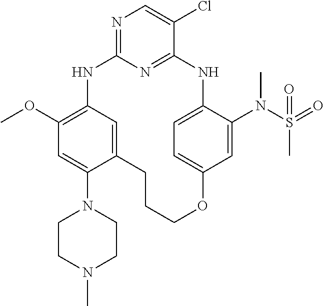 [SMILES: COc1cc(N2CCN(C)CC2)c2cc1Nc1ncc(Cl)c(n1)Nc1ccc(cc1N(C)S(C)(=O)=O)OCCC2]

Following other similar steps as in Example A1 to give Example A2.

### Example A5

### N-(3⁵-chloro-1⁴-((2-(dimethylamino)ethyl)(methyl)amino)-1⁶-methoxy-6-oxa-2,4-diaza-3(2,4)-pyrimidina-1(1,3),5(1,4)-dibenzenacycloundecaphane-5²-yl)-N-methylmethanesulfonamide

### Step A: 5-(2 fluoro-4-methoxy-5-nitrophenyl)pent-4-yn-1-ol

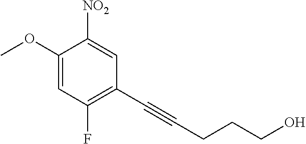 [SMILES: COc1cc(F)c(C#CCCCO)cc1[N+](=O)[O-]]

1-bromo-2-fluoro-4-methoxy-5-nitrobenzene (1.0 g, 4 mmol), (PPh₃)₂PdCl₂ (280 mg, 0.4 mmol), CuI (167 mg, 0.8 mmol), triphenylphosphine (213 mg, 0.8 mmol) was added into a 100 ml Schlenk tube and dissolved in 10 ml dry DMF. The mixture was degassed and refilled with argon. Then pent-4-yn-1-ol (504 mg, 6 mmol) and diisopropylethylamine (2.5 ml, 20 mmol) was added under argon. The mixture was stirred at 80° C. under argon overnight and used for next step without purification. LCMS m/z 254.2 (M+H)⁺.

### Step B: 5-(2-((2-(dimethylamino)ethyl)(methyl)amino)-4-methoxy-5-nitrophenyl)pent-4-yn-1-ol

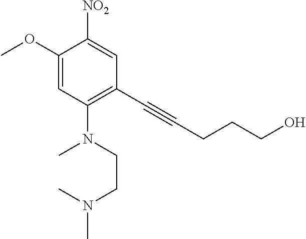 [SMILES: COc1cc(N(C)CCN(C)C)c(C#CCCCO)cc1[N+](=O)[O-]]

To the DMF solution of crude 5-(2 fluoro-4-methoxy-5-nitrophenyl)pent-4-yn-1-ol obtained from step A was added potassium carbonate (1.68 g, 12 mmol) and N,N,N′-Trimethylethylenediamine (510 mg, 5 mmol). The mixture was heated to 80° C. for one hour. Water (10 mL) was added to quench the reaction, the product was extracted with chloroform/isopropanol=3:1 and purified by column chromatography on silica gel using MeOH/dichloromethane (10/90) (800 mg, 60% yield for two steps). LCMS m/z 336.4 (M+H)⁺.

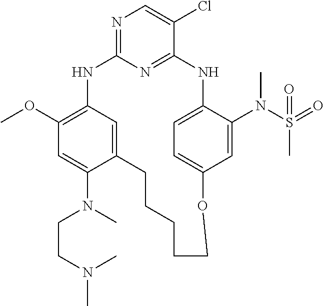 [SMILES: COc1cc(N(C)CCN(C)C)c2cc1Nc1ncc(Cl)c(n1)Nc1ccc(cc1N(C)S(C)(=O)=O)OCCCCC2]

Following similar methods as for Examples A2, Example A5 was prepared.

### Example A18

### (3⁵-chloro-1⁶-methoxy-1⁴-(4-methyl piperazin-1-yl)-6-oxa-2,4-diaza-3(2,4)-pyrimidina-1(1,3),5(1,4)-dibenzenacycloundecaphane-5²-yl)dimethylphosphine oxide

### Step A: (2-amino-5-methoxyphenyl)dimethylphosphine Oxide

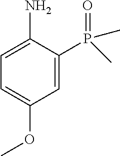

To a solution of 2-iodo-4-methoxyaniline (1.25 g, 5 mmol), dimethyl phosphite (760 mg, 7.5 mmol) and potassium phosphate (1.2 g, 7.5 mmol) in DMF (25 mL) under nitrogen atmosphere, Pd(OAc)₂ (60 mg, 5%) and Xantphos (150 mg) was added. The reaction mixture was degassed and purged with nitrogen and stirred at 120° C. overnight. Water (10 mL) was added to quench the reaction, and the mixture was extracted with chloroform/isopropanol=3:1. The organic layers were collected, dried over sodium sulfate and concentrated under vacuum to give crude product, further purified by column chromatography on silica gel using hexane/ethyl acetate (70/30) (520 mg, 52% yield). LCMS m/z 201.2 (M+H)⁺.

### Step B: (2-((2,5-dichloropyrimidin-4-yl)amino)-5-methoxyphenyl)dimethylphosphine oxide

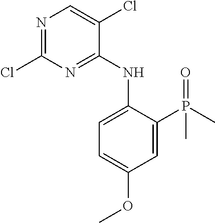 [SMILES: COc1ccc(Nc2nc(Cl)ncc2Cl)c(P(C)(C)=O)c1]

To a solution of (2-amino-5-methoxyphenyl)dimethylphosphine oxide (500 mg, 2.5 mmol) in dry DMF (10 ml) was added sodium hydride (60%, 200 mg, 5 mmol) at 0° C. and stirred for about 30 minutes. 2,4,5-trichloropyrimidine (550 mg, 3 mmol) was added and the mixture was gradually warmed to room temperature and stirred overnight. Water was added to quench the reaction, and the mixture was extracted with chloroform/isopropanol=3:1. The organic layers were collected, dried over sodium sulfate and concentrated under vacuum to give crude product, further purified by column chromatography on silica gel using dichloromethane/methanol (90/10) (350 mg, 41% yield). LCMS m/z 348.2 (M+H)⁺.

### Step C: (2-((2,5-dichloropyrimidin-4-yl)amino)-5-hydroxyphenyl)dimethylphosphine Oxide

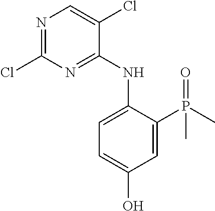 [SMILES: CP(C)(=O)c1cc(O)ccc1Nc1nc(Cl)ncc1Cl]

To a solution of (2-((2,5-dichloropyrimidin-4-yl)amino)-5-methoxyphenyl)dimethylphosphine oxide (347 mg, 1 mmol) in dichloromethane (5 ml) was added 2M BBr₃ in dichloromethane (5 ml, 10 mmol) at 0° C. The mixture was gradually warmed to room temperature and stirred overnight. Saturated aqueous NaHCO₃ solution was added to quench the reaction. The mixture was extracted with CHCl₃/isopropanol (3/1) mixture and the crude product was purified by column chromatography on silica gel using MeOH/dichloromethane (10/90) (300 mg, 90% yield). LCMS m/z 334.2 (M+H)⁺.

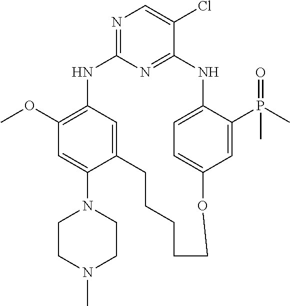

Using similar methods as for Examples A1, Example A18 was prepared.

### Example A21

### N-(3⁵-chloro-1⁶-ethoxy-1⁴-(4-methylpiperazin-1-yl)-6-oxa-2,4-diaza-3(2,4)-pyrimidina-1(1,3),5(1,4)-dibenzenacycloundecaphane-5²-yl)-N-methylmethanesulfonamide

### Step A: 4-bromo-5 fluoro-2-nitrophenol

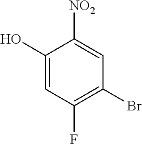 [SMILES: O=[N+]([O-])c1cc(Br)c(F)cc1O]

To a stirring solution of 4-bromo-3-fluorophenol (10.23 g, 53.56 mmol) in dichloromethane (108 mL) at 0° C., concentrated sulfuric acid (6 ml, 107 mmol) was added followed by nitric acid (65%, 3.8 ml, 53.6 mmol) at 0° C. After one hour stirring, the mixture was quenched with ice water, extracted three times with dichloromethane. The combined organic layers were collected and concentrated under vacuum. The resulting residue was purified by column chromatography on silica gel using hexane/ethyl acetate (90/10) (6.7 g, 53% yield). LCMS m/z 237.2 (M+H)⁺.

### Step B: 1-bromo-4-ethoxy-2 fluoro-5-nitrobenzene

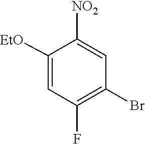 [SMILES: CCOc1cc(F)c(Br)cc1[N+](=O)[O-]]

4-bromo-5-fluoro-2-nitrophenol (1 g, 4.2 mmol) and potassium carbonate (1.17 g, 8.4 mmol) was dissolved in DMF (10 ml). Ethyl iodide (0.68 ml, 8.4 mmol) was added and the mixture was heated to 60° C. and stirred overnight. Water (10 mL) was added to quench the reaction, and the mixture was extracted with chloroform/isopropanol=3:1. The organic layers were collected, dried over sodium sulfate and concentrated under vacuum to give crude product, further purified by column chromatography on silica gel using hexane/ethyl acetate (80/20) (750 mg, 68% yield). LCMS m/z 265.4 (M+H)⁺.

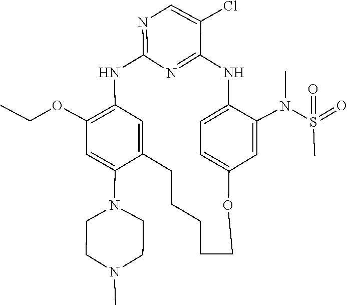 [SMILES: CCOc1cc(N2CCN(C)CC2)c2cc1Nc1ncc(Cl)c(n1)Nc1ccc(cc1N(C)S(C)(=O)=O)OCCCCC2]

Using similar methods as for Examples A2, Example A21 was prepared.

### Example A22

### N-(3⁵-chloro-1⁶-isopropoxy-1⁴-(4-methyl piperazin-1-yl)-6-oxa-2,4-diaza-3(2,4)-pyrimidina-1(1,3),5(1,4)-dibenzenacycloundecaphane-5²-yl)-N-methylmethanesulfonamide

### Step A: 1-bromo-2 fluoro-4-isopropoxy-5-nitrobenzene

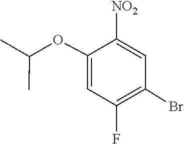

To a mixture of 4-bromo-5-fluoro-2-nitrophenol (1 g, 4.2 mmol) and potassium carbonate (1.17 g, 8.4 mmol) in DMF (10 ml), isopropyl bromide (0.8 ml, 8.4 mmol) was added. The mixture was heated to 60° C. and stirred overnight. water (20 mL) was added and the mixture was extracted with chloroform/isopropanol=3:1. The organic layers were collected, dried over sodium sulfate and concentrated under vacuum to give crude product, further purified by column chromatography on silica gel using hexane/ethyl acetate (80/20) (850 mg, 73% yield). (M+H)⁺: 279.2.

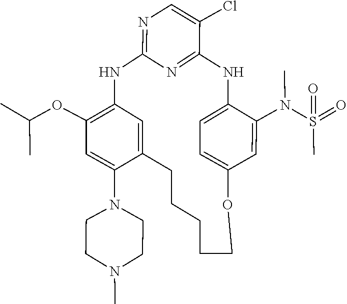 [SMILES: CC(C)Oc1cc(N2CCN(C)CC2)c2cc1Nc1ncc(Cl)c(n1)Nc1ccc(cc1N(C)S(C)(=O)=O)OCCCCC2]

Using similar methods as for Examples A2, Example A22 was prepared.

### Example A25

### 3⁵-chloro-1⁶-methoxy-N,N-dimethyl-1⁴-(4-methylpiperazin-1-yl)-6-oxa-2,4-diaza-3(2,4)-pyrimidina-1(1,3),5(1,4)-dibenzenacyclododecaphane-5²-carboxamide

### Step A: 5-methoxy-N,N-dimethyl-2-nitrobenzamide

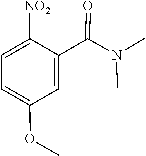 [SMILES: COc1ccc([N+](=O)[O-])c(C(=O)N(C)C)c1]

To a stirring solution of 5-methoxy-2-nitrobenzoic acid (1.97 g, 10 mmol) in dichloromethane (50 ml) at 0° C. was added oxalyl chloride (1.5 ml, 40 mmol) and catalytic amount of DMF. The reaction was gradually warmed to room temperature and stirred overnight. Upon completion, solvent and excess oxalyl chloride were evaporated under vacuum to give crude acid chloride which is used directly without purification. The crude acid chloride was dissolved in dry dichloromethane (50 ml), then dimethylamine hydrochloride (984 mg, 12 mmol) and triethylamine (2.1 ml, 20 mmol) were added to the mixture and stirred overnight. Water (15 ml) was added and the mixture was extracted with chloroform/isopropanol=3:1. The organic layers were collected, dried over sodium sulfate and concentrated under vacuum to give crude product, further purified by column chromatography on silica gel using hexane/ethyl acetate (1.2 g, 54% yield). LCMS m/z 225.3 (M+H)⁺.

### Step B: 2-amino-5-methoxy-N,N-dimethylbenzamide

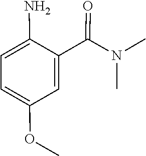

To a solution of 5-methoxy-N,N-dimethyl-2-nitrobenzamide (1.2 g, 5.4 mmol) in methanol (50 ml) was added 10% Pd/C (50 mg). The reaction mixture was degassed, refilled with hydrogen gas, and stirred to react overnight at room temperature. The mixture was filtered through Celite and washed with methanol. The combined filtrate was collected and concentrated under vacuum to afford the crude product, which was purified by column chromatography on silica gel using MeOH/dichloromethane (10/90) (1 g, 95% yield). LCMS m/z 195.3 (M+H)⁺.

### Step C: 2-((2,5-dichloropyrimidin-4-yl)amino)-5-methoxy-N,N-dimethylbenzamide

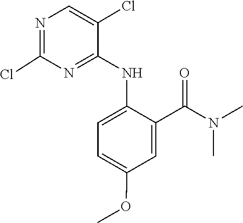 [SMILES: COc1ccc(Nc2nc(Cl)ncc2Cl)c(C(=O)N(C)C)c1]

To a solution of 2-amino-5-methoxy-N,N-dimethylbenzamide (970 mg, 5 mmol) in dry DMF (20 ml) was added sodium hydride (60%, 400 mg, 10 mmol) at 0° C. and stir for about 30 minutes. 2,4,5-trichloropyrimidine (1.1 g, 6 mmol) was added and the mixture was gradually warmed to room temperature and stirred overnight. Water was added to quench the reaction, and the mixture was extracted with chloroform/isopropanol=3:1. The organic layers were collected, dried over sodium sulfate and concentrated under vacuum to give crude product, further purified by column chromatography on silica gel using hexane/ethyl acetate (70/30) (720 mg, 42% yield). LCMS m/z 342.2 (M+H)⁺.

### Step D: 2-((2,5-dichloropyrimidin-4-yl)amino)-5-hydroxy-N,N-dimethylbenzamide

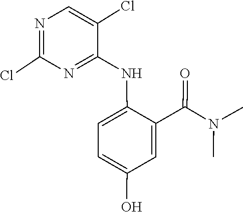 [SMILES: CN(C)C(=O)c1cc(O)ccc1Nc1nc(Cl)ncc1Cl]

To a solution of 2-((2,5-dichloropyrimidin-4-yl)amino)-5-methoxy-N,N-dimethylbenzamide (682 mg, 2 mmol) in dichloromethane (10 ml) was added 2M BBr₃ in dichloromethane (10 ml, 20 mmol) at 0° C. The mixture was gradually warmed to room temperature and stirred overnight. Saturated aqueous NaHCO₃ solution was added to quench the reaction. The mixture was extracted with CHCl₃/isopropanol (3/1) mixture and the crude product was purified by column chromatography on silica gel using MeOH/dichloromethane (380 mg, 58% yield). LCMS m/z 327.2 (M+H)⁺.

### Step E: Tert-Butyl (5-(5-hydroxypentyl)-2-methoxy-4-(4-methylpiperazin-1-yl)phenyl)carbamate

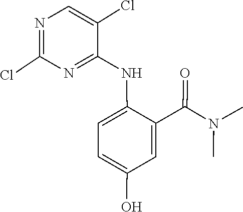 [SMILES: CN(C)C(=O)c1cc(O)ccc1Nc1nc(Cl)ncc1Cl]

To a solution of 5-(5-amino-4-methoxy-2-(4-methylpiperazin-1-yl)phenyl)pentan-1-ol (322 mg, 1 mmol) and triethylamine (202 mg, 2 mmol) in dioxane (10 ml) was added di-tert-butyl dicarbonate (436 mg, 2 mmol). The reaction mixture was heated to 90° C. and stirred overnight. The mixture was concentrated under vacuum to afford the crude product, which was purified by column chromatography on silica gel using MeOH/dichloromethane (340 mg, 80% yield). LCMS m/z 424.4 (M+H)⁺.

### Step F: Tert-Butyl (5-(5-(4-((2,5-dichloropyrimidin-4-yl)amino)-3-(dimethylcarbamoyl)phenoxy)pentyl)-2-methoxy-4-(4-methylpiperazin-1-yl)phenyl)carbamate

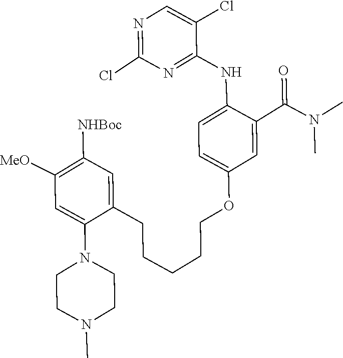 [SMILES: COc1cc(N2CCN(C)CC2)c(CCCCCOc2ccc(Nc3nc(Cl)ncc3Cl)c(C(=O)N(C)C)c2)cc1NC(=O)OC(C)(C)C]

To a solution of 2-((2,5-dichloropyrimidin-4-yl)amino)-5-hydroxy-N,N-dimethylbenzamide (163 mg, 0.5 mmol), tert-butyl (5-(5-hydroxypentyl)-2-methoxy-4-(4-methylpiperazin-1-yl)phenyl)carbamate (212 mg, 0.5 mmol) and triphenylphosphine (262 mg, 1 mmol) in anhydrous THE (5 ml) was added DIAD (202 mg, 1 mmol) at 0° C. The mixture was gradually warmed to room temperature and stirred overnight. The mixture was concentrated under vacuum and purified by column chromatography on silica gel using MeOH/dichloromethane (10/90) (150 mg, 41% yield). LCMS m/z 733.3 (M+H)⁺.

### Step G: 5-((5-(5-amino-4-methoxy-2-(4-methylpiperazin-1-yl)phenyl)pentyl)oxy)-2-((2,5-dichloropyrimidin-4-yl)amino)-N,N-dimethylbenzamide

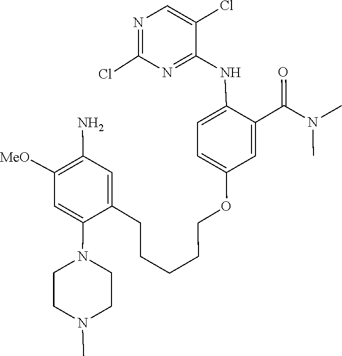 [SMILES: COc1cc(N2CCN(C)CC2)c(CCCCCOc2ccc(Nc3nc(Cl)ncc3Cl)c(C(=O)N(C)C)c2)cc1N]

To a solution of tert-butyl (5-(5-(4-((2,5-dichloropyrimidin-4-yl)amino)-3-(dimethylcarbamoyl)phenoxy)pentyl)-2-methoxy-4-(4-methylpiperazin-1-yl)phenyl)carbamate (150 mg, 0.21 mmol) in DCM (1 ml) was added TFA (3 ml) at 0° C. The mixture was gradually warmed to room temperature and stirred overnight. The mixture was concentrated under vacuum and the residue was used for next step without further purification. LCMS m/z 633.3 (M+H)⁺.

### Step G: 3⁵-chloro-1⁶-methoxy-N, N-dimethyl-1⁴-(4-methylpiperazin-1-yl)-6-oxa-2,4-diaza-3(2,4)-pyrimidina-1(1,3),5(1,4)-dibenzenacyclododecaphane-5²-carboxamide

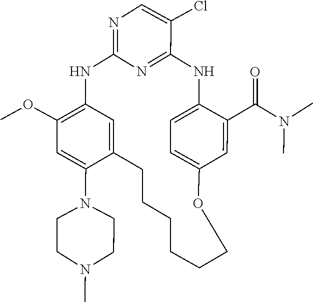

(5-(5-(4-((2,5-Dichloropyrimidin-4-yl)amino)-3-(dimethylcarbamoyl)phenoxy)pentyl)-2-methoxy-4-(4-methylpiperazin-1-yl)phenyl)carbamate (126 mg, 0.2 mmol), cesium carbonate (260 mg, 0.8 mmol), Pd₂(dba)₃ (9.2 mg, 5%) and XPhos (9.5 mg, 10%) were mixed in a Schlenk tube with anhydrous DMF (3 ml). The mixture was degassed and refilled with argon. Then the mixture was heated to 80° C. and stirred vigorously overnight. Water was added to quench the reaction, and the mixture was extracted with chloroform/isopropanol=3:1. The organic layers were collected, dried over sodium sulfate and concentrated under vacuum to give crude product which was further purified by column chromatography on silica gel using dichloromethane/methanol (90/10).

### Example A29

### N-(3⁵-bromo-1⁶-methoxy-1⁴-(4-(4-methylpiperazin-1-yl)piperidin-1-yl)-6-oxa-2,4-diaza-3(2,4)-pyrimidina-1(1,3),5(1,4)-dibenzenacycloundecaphane-5²-yl)-N-methylmethanesulfonamide

### Step A: N-(2-((5-bromo-2-chloropyrimidin-4-yl)amino)-5-methoxyphenyl)-N-methylmethanesulfonamide

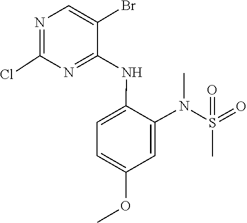 [SMILES: COc1ccc(Nc2nc(Cl)ncc2Br)c(N(C)S(C)(=O)=O)c1]

To a solution of N-(2-amino-5-methoxyphenyl)-N-methylmethanesulfonamide (2.3g, 10 mmol) in iPrOH (50 ml) was added 5-bromo-2,4-dichloropyrimidine (3.42g, 15 mmol) and diisopropylethylamine (10 ml). The mixture was heated to reflux overnight. Solvent was removed under reduced pressure, and the residue was purified by column chromatography on silica gel using hexane/ethyl acetate (50:50) (3.2g, 76% yield). LCMS m/z 423.40 (M+H)⁺.

### Step B: N-(2-((5-bromo-2-chloropyrimidin-4-yl)amino)-5-hydroxyphenyl)-N-methylmethanesulfonamide

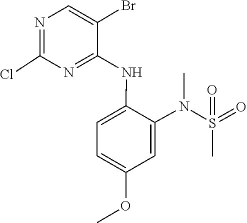 [SMILES: COc1ccc(Nc2nc(Cl)ncc2Br)c(N(C)S(C)(=O)=O)c1]

To a solution of N-(2-((5-bromo-2-chloropyrimidin-4-yl)amino)-5-methoxyphenyl)-N-methylmethanesulfonamide (3.2g, 7.6 mmol) in dichloromethane (20 ml) was added 2M BBr₃ in dichloromethane (20 ml, 40 mmol) at 0° C. The mixture was gradually warmed to room temperature and stirred overnight. Saturated aqueous NaHCO₃ solution was added to quench the reaction. The mixture was extracted with CHCl₃/isopropanol (3/1) mixture and the crude product was purified by column chromatography on silica gel using MeOH/dichloromethane (10/90) (2.5g, 81% yield). LCMS m/z 409.18 (M+H)⁺.

### Step C: N-(2-((5-bromo-2-((5-(5-hydroxypentyl)-2-methoxy-4-(4-(4-methylpiperazin-1-yl)piperidin-1-yl)phenyl)amino)pyrimidin-4-yl)amino)-5-hydroxyphenyl)-N-methylmethanesulfonamide

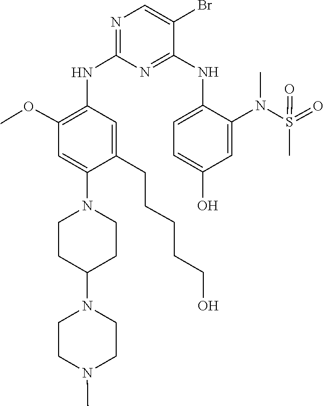 [SMILES: COc1cc(N2CCC(N3CCN(C)CC3)CC2)c(CCCCCO)cc1Nc1ncc(Br)c(Nc2ccc(O)cc2N(C)S(C)(=O)=O)n1]

To a solution of N-(2-((5-bromo-2-chloropyrimidin-4-yl)amino)-5-hydroxyphenyl)-N-methylmethanesulfonamide (1.22 g, 3 mmol) and 5-(5-amino-4-methoxy-2-(4-(4-methylpiperazin-1-yl)piperidin-1-yl)phenyl)pentan-1-ol (975 mg, 2.5 mmol) in 2-methoxyethanol (20 ml) was added methanesulfonic acid (720 mg, 7.5 mmol). The mixture was stirred at 90° C. overnight. Saturated NaHCO₃ aqueous solution (20 mL) was added to quench the reaction, and the product was extracted with chloroform/isopropanol=3:1 and purified by column chromatography on silica gel using MeOH/dichloromethane (10/90) (1.45 g, 76% yield). LCMS m/z 763.73 (M+H)⁺.

### Step D: N-(2-((5-bromo-2-((5-(5-bromopentyl)-2-methoxy-4-(4-(4-methylpiperazin-1-yl)piperidin-1-yl)phenyl)amino)pyrimidin-4-yl)amino)-5-hydroxyphenyl)-N-methylmethanesulfonamide

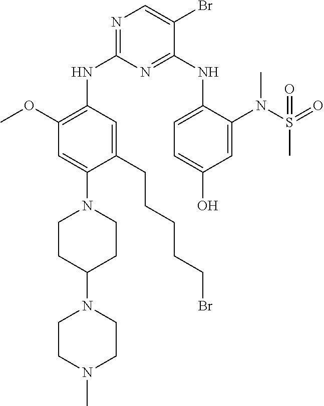 [SMILES: COc1cc(N2CCC(N3CCN(C)CC3)CC2)c(CCCCCBr)cc1Nc1ncc(Br)c(Nc2ccc(O)cc2N(C)S(C)(=O)=O)n1]

To a solution of N-(2-((5-bromo-2-((5-(5-hydroxypentyl)-2-methoxy-4-(4-(4-methylpiperazin-1-yl)piperidin-1-yl)phenyl)amino)pyrimidin-4-yl)amino)-5-hydroxyphenyl)-N-methylmethanesulfonamide (700 mg, 0.092 mmol) in a mixture of CHCl₃(20 ml) and pyridine (6 ml) was added triphenylphosphine (1.44 g, 5.5 mmol) and CBr₄ (1.82 g, 5.5 mmol). The mixture was stirred overnight at room temperature. Water (20 ml) was added and the product was extracted with CHCl₃/isopropanol (3/1). The crude product was purified by column chromatography on silica gel using Acetonitrile/water (80/20) (500 mg, 66% yield). LCMS m/z 825.65 (M+H)⁺.

### Step E: N-(3⁵-bromo-1⁶-methoxy-1⁴-(4-(4-methylpiperazin-1-yl)piperidin-1-yl)-6-oxa-2,4-diaza-3(2,4)-pyrimidina-1(1,3), 5(1,4)-dibenzenacycloundecaphane-5²-yl)-N-methylmethanesulfonamide

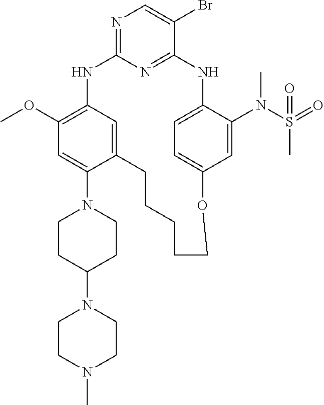 [SMILES: COc1cc(N2CCC(N3CCN(C)CC3)CC2)c2cc1Nc1ncc(Br)c(n1)Nc1ccc(cc1N(C)S(C)(=O)=O)OCCCCC2]

To a solution of N-(2-((5-bromo-2-((5-(5-bromopentyl)-2-methoxy-4-(4-(4-methylpiperazin-1-yl)piperidin-1-yl)phenyl)amino)pyrimidin-4-yl)amino)-5-hydroxyphenyl)-N-methylmethanesulfonamide (500 mg, 0.61 mmol) in anhydrous DMF (60 ml) was added potassium iodide (30 mg, 0.18 mmol) and potassium carbonate (336 mg, 2.4 mmol). The mixture was stirred overnight at room temperature. Most of the DMF was removed on rotavapor and water (20 ml) was added. The product was extracted with dichloromethane. The crude product was purified by column chromatography on silica gel using dichloromethane/2M ammonia in methanol (95/5) (250 mg, 55% yield).

### Example A39

### N-(3⁵-chloro-1⁶-methoxy-1⁴-(4-(4-methylpiperazin-1-yl)piperidin-1-yl)-10-oxo-6-oxa-2,4,11-triaza-3(2,4)-pyrimidina-1(1,3),5(1,4)-dibenzenacycloundecaphane-5²-yl)-N-methylmethanesulfonamide

### Step A: 2-methoxy-4-(4-(4-methylpiperazin-1-yl)piperidin-1-yl)-5-nitroaniline

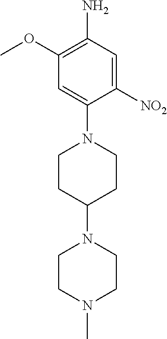 [SMILES: COc1cc(N2CCC(N3CCN(C)CC3)CC2)c([N+](=O)[O-])cc1N]

To a solution of 4-fluoro-2-methoxy-5-nitroaniline (744 mg, 4 mmol) in DMF (10 ml) was added 1-methyl-4-(piperidin-4-yl)piperazine trihydrochloride (1.17 g, 4 mmol) and potassium carbonate (2.8g, 20 mmol). The mixture was stirred at 80° C. overnight. Water (10 ml) was added and the product was extracted with dichloromethane. The crude product was purified by column chromatography on silica gel using dichloromethane/methanol (90/10) (1.05 g, 75% yield). LCMS m/z 350.32 (M+H)⁺.

### Step B: N-(2-((5-chloro-2-((2-methoxy-4-(4-(4-methylpiperazin-1-yl)piperidin-1-yl)-5-nitrophenyl)amino)pyrimidin-4-yl)amino)-5-hydroxyphenyl)-N-methylmethanesulfonamide

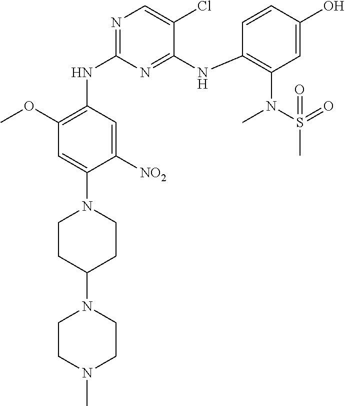 [SMILES: COc1cc(N2CCC(N3CCN(C)CC3)CC2)c([N+](=O)[O-])cc1Nc1ncc(Cl)c(Nc2ccc(O)cc2N(C)S(C)(=O)=O)n1]

To a solution of N-(2-((2,5-dichloropyrimidin-4-yl)amino)-5-hydroxyphenyl)-N-methylmethanesulfonamide (726 mg, 2 mmol) and 2-methoxy-4-(4-(4-methylpiperazin-1-yl)piperidin-1-yl)-5-nitroaniline (768 mg, 2.2 mmol) in 2-methoxyethanol (10 ml) was added methanesulfonic acid (576 mg, 6 mmol). The mixture was stirred at 90° C. overnight. Saturated NaHCO₃ aqueous solution (10 mL) was added, and the product was extracted with chloroform/isopropanol=3:1 and purified by column chromatography on silica gel using MeOH/dichloromethane (10/90) (500 mg, 37% yield). LCMS m/z 676.56 (M+H)⁺.

### Step C: N-(2-((2-((5-amino-2-methoxy-4-(4-(4-methylpiperazin-1-yl)piperidin-1-yl)phenyl)amino)-5-chloropyrimidin-4-yl)amino)-5-hydroxyphenyl)-N-methylmethanesulfonamide

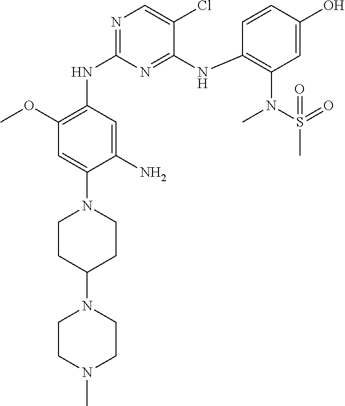 [SMILES: COc1cc(N2CCC(N3CCN(C)CC3)CC2)c(N)cc1Nc1ncc(Cl)c(Nc2ccc(O)cc2N(C)S(C)(=O)=O)n1]

To a solution of N-(2-((5-chloro-2-((2-methoxy-4-(4-(4-methylpiperazin-1-yl)piperidin-1-yl)-5-nitrophenyl)amino)pyrimidin-4-yl)amino)-5-hydroxyphenyl)-N-methylmethanesulfonamide (500 mg, 0.74 mmol) in acetone (5 ml) and water (2 ml) was added ammonium chloride (400 mg, 7.4 mmol) and zinc (264 mg, 4.4 mmol) at 0° C. The mixture was stirred at room temperature for about 10 minutes, filtered through Celite, washed with acetone. The filtrate was concentrated under vacuum and was extracted with CHCl₃/isopropanol (3/1). After washing with brine and dried over sodium sulfate, the solvent was removed to give a dark solid. Directly used in the next step without further purifications. (340 mg, 71% yield). LCMS m/z 546.47 (M+H)⁺.

### Step D: 4-bromo-N-(5-((5-chloro-4-((4-hydroxy-2-(N-methylmethylsulfonamido)phenyl)amino)pyrimidin-2-yl)amino)-4-methoxy-2-(4-(4-methylpiperazin-1-yl)piperidin-1-yl)phenyl)butanamide

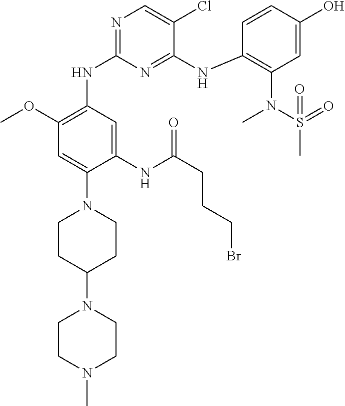 [SMILES: COc1cc(N2CCC(N3CCN(C)CC3)CC2)c(NC(=O)CCCBr)cc1Nc1ncc(Cl)c(Nc2ccc(O)cc2N(C)S(C)(=O)=O)n1]

To a solution of N-(2-((2-((5-amino-2-methoxy-4-(4-(4-methylpiperazin-1-yl)piperidin-1-yl)phenyl)amino)-5-chloropyrimidin-4-yl)amino)-5-hydroxyphenyl)-N-methylmethanesulfonamide (65 mg, 0.1 mmol) in dichloromethane (5 ml) was added diisopropylethylamine (26 mg, 0.2 mmol) and 4-bromobutanoyl chloride (20 mg, 0.11 mmol) at 0° C. The mixture was stirred at room temperature for about one hour. Water (10 ml) was added, and the crude was extracted with dichloromethane. After washing with brine and dried over sodium sulfate, the solvent was removed to give a gray solid. Directly used in the next step without further purifications. (81 mg, crude). LCMS m/z 796.4 (M+H)⁺.

### Step E: N-(3⁵-chloro-1⁶-methoxy-1⁴-(4-(4-methylpiperazin-1-yl)piperidin-1-yl)-10-oxo-6-oxa-2,4,11-triaza-3(2,4)-pyrimidina-1(1,3),5(1,4)-dibenzenacycloundecaphane-5²-yl)-N-methylmethanesulfonamide

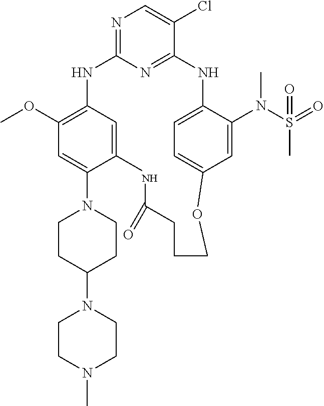 [SMILES: COc1cc(N2CCC(N3CCN(C)CC3)CC2)c2cc1Nc1ncc(Cl)c(n1)Nc1ccc(cc1N(C)S(C)(=O)=O)OCCCC(=O)N2]

To a solution of 4-bromo-N-(5-((5-chloro-4-((4-hydroxy-2-(N-methylmethylsulfonamido)phenyl)amino)pyrimidin-2-yl)amino)-4-methoxy-2-(4-(4-methylpiperazin-1-yl)piperidin-1-yl)phenyl)butanamide (81 mg, 0.1 mmol) in DMF (5 ml) was added potassium iodide (17 mg, 0.1 mmol) and potassium carbonate (28 mg, 0.2 mmol). The mixture was stirred at room temperature overnight. Water (10 ml) was added, and the crude was extracted with dichloromethane, purified by column chromatography on silica gel using dichloromethane/2M ammonia methanol solution (10/90) (3 mg).

### Example A41

### N-(3⁵-bromo-1⁶-methoxy-1⁴-(4-(4-methylpiperazin-1-yl)piperidin-1-yl)-8-oxo-2,4,9-triaza-3(2,4)-pyrimidina-1(1,3),5(1,4)-dibenzenacyclododecaphane-5²-yl)-N-methylmethanesulfonamide

### Step A: N-(5-bromo-2-nitrophenyl)-N-methylmethanesulfonamide

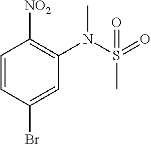 [SMILES: CN(c1cc(Br)ccc1[N+](=O)[O-])S(C)(=O)=O]

To a suspension of cesium carbonate (6.2 g, 20 mmol) in acetonitrile (100 ml) at room temperature was added N-methylmethanesulfonamide (3.28 g, 20 mmol) and 4-bromo-2-fluoro-1-nitrobenzene (4.4 g, 20 mmol) dropwise over 15 minutes. The reaction was stirred overnight. Upon completion, the mixture was filtered and then concentrated. The residue was purified by column chromatography on silica gel using MeOH/dichloromethane (10/90) to afford the product (5.6 g, 91% yield). LCMS m/z 311.09 (M+H)⁺.

### Step B: Tert-Butyl 3-(3-(N-methylmethylsulfonamido)-4-nitrophenyl)acrylate

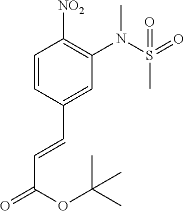 [SMILES: CN(c1cc(/C=C/C(=O)OC(C)(C)C)ccc1[N+](=O)[O-])S(C)(=O)=O]

N-(5-bromo-2-nitrophenyl)-N-methylmethanesulfonamide (620 mg, 2 mmol), ter-butyl acrylate (770 mg, 6 mmol), (PPh₃)₂PdCl₂ (45 mg, 0.2 mmol), triphenylphosphine (105 mg, 0.4 mmol), triethylamine (606 mg, 6 mmol) and dry DMF (10 ml) were added into a 100 ml Schlenk tube. The mixture was degassed and refilled with argon. The mixture was stirred at 80° C. under argon overnight. Water (10 ml) was added and the mixture was extracted with dichloromethane. The crude product was purified by column chromatography on silica gel using dichloromethane/acetonitrile (90/10) to afford the product (350 mg, 49% yield). LCMS m/z 357.21 (M+H)⁺.

### Step C: Tert-Butyl 3-(4-amino-3-(N-methylmethylsulfonamido)phenyl)propanoate

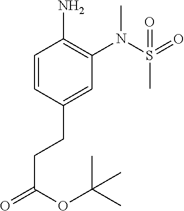 [SMILES: CN(c1cc(CCC(=O)OC(C)(C)C)ccc1N)S(C)(=O)=O]

To a solution of tert-butyl 3-(3-(N-methylmethylsulfonamido)-4-nitrophenyl)acrylate (300 mg, 1 mmol) in methanol (50 ml) was added 10% Pd/C (50 mg). The reaction mixture was degassed, refilled with hydrogen gas, and stirred overnight at room temperature. After the reaction was completed, the mixture was filtered through Celite and washed with methanol. The combined filtrate was concentrated under vacuum to afford the crude product, which was used without further purification (300 mg, 91% yield). LCMS m/z 329.27 (M+H)⁺.

### Step D: N-(2-((5-bromo-2-chloropyrimidin-4-yl)amino)-5-methoxyphenyl)-N-methylmethanesulfonamide

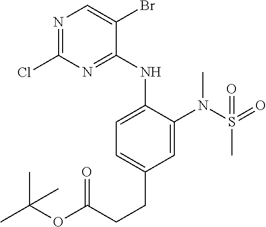 [SMILES: CN(c1cc(CCC(=O)OC(C)(C)C)ccc1Nc1nc(Cl)ncc1Br)S(C)(=O)=O]

To a solution of tert-butyl 3-(4-amino-3-(N-methylmethylsulfonamido)phenyl)propanoate (300 mg, 0.9 mmol) in iPrOH (15 ml) was added 5-bromo-2,4-dichloropyrimidine (251 mg, 1.1 mmol) and diisopropylethylamine (10 ml). The mixture was heated to reflux for 3 days. Solvent was removed under reduced pressure, and the residue was purified by column chromatography on silica gel using dichloromethane/acetonitrile (94/6) (370 mg, 78% yield). LCMS m/z 521.29 (M+H)⁺.

### Step E: 3-(4-((5-bromo-2-chloropyrimidin-4-yl)amino)-3-(N-methylmethylsulfonamido)phenyl)propanoic Acid

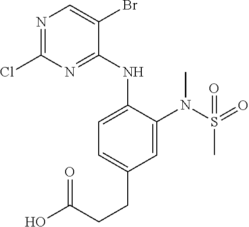 [SMILES: CN(c1cc(CCC(=O)O)ccc1Nc1nc(Cl)ncc1Br)S(C)(=O)=O]

To a solution of tert-butyl N-(2-((5-bromo-2-chloropyrimidin-4-yl)amino)-5-methoxyphenyl)-N-methylmethanesulfonamide (75 mg, 0.15 mmol) in dioxane (3 ml) was hydrochloride in dioxane (4 N, 3 ml, 12 mmol). The mixture was stirred at room temperature overnight. Solvent was removed under reduced pressure, and the residue was used directly in the next step without further purification. LCMS m/z 465.19 (M+H)⁺.

### Step F: tert-butyl (3-(2 fluoro-4-methoxy-5-nitrophenyl)prop-2-yn-1-yl)carbamate

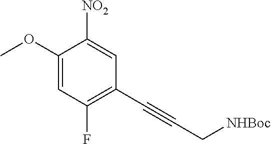 [SMILES: COc1cc(F)c(C#CCNC(=O)OC(C)(C)C)cc1[N+](=O)[O-]]

1-Bromo-2-fluoro-4-methoxy-5-nitrobenzene (1.0 g, 4 mmol), CuI (152 mg, 0.8 mmol), diisopropylethylamine (2.58 g, 20 mmol) and dry DMF (10 ml) were added into a 100 ml Schlenk tube. Nitrogen was bubbled through the mixture for about 10 minutes. (PPh₃)₂PdCl₂ (281 mg, 0.4 mmol) was added and the mixture was stirred at 80° C. under nitrogen overnight. Water (10 ml) was added and the mixture was extracted with dichloromethane. The crude product was purified by column chromatography on silica gel using dichloromethane/acetonitrile (97/3) to afford the product (580 mg, 45% yield). LCMS m/z 326.18 (M+H)⁺.

### Step G: Tert-Butyl (3-(4-methoxy-2-(4-(4-methylpiperazin-1-yl)piperidin-1-yl)-5-nitrophenyl)prop-2-yn-1-yl)carbamate

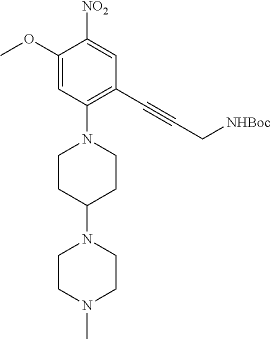 [SMILES: COc1cc(N2CCC(N3CCN(C)CC3)CC2)c(C#CCNC(=O)OC(C)(C)C)cc1[N+](=O)[O-]]

To a solution of tert-butyl (3-(2-fluoro-4-methoxy-5-nitrophenyl)prop-2-yn-1-yl)carbamate (580 mg, 1.8 mmol) in DMF (10 ml) was added 1-methyl-4-(piperidin-4-yl)piperazine trihydrochloride (484 mg, 2.2 mmol) and potassium carbonate (1.25 g, 9 mmol). The mixture was stirred at 80° C. overnight. Water (10 ml) was added and the product was extracted with dichloromethane. The crude product was used directly in the next step without further purification (800 mg, 91% yield). LCMS m/z 488.42 (M+H)⁺.

### Step H: Tert-Butyl (3-(5-amino-4-methoxy-2-(4-(4-methylpiperazin-1-yl)piperidin-1-yl)phenyl)propyl)carbamate

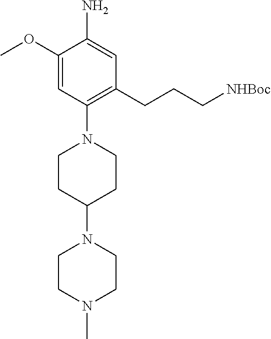 [SMILES: COc1cc(N2CCC(N3CCN(C)CC3)CC2)c(CCCNC(=O)OC(C)(C)C)cc1N]

To a solution of tert-butyl (3-(4-methoxy-2-(4-(4-methylpiperazin-1-yl)piperidin-1-yl)-5-nitrophenyl)prop-2-yn-1-yl)carbamate (400 mg, 0.82 mmol) in methanol (10 ml) was added 10% Pd/C (50 mg). The reaction mixture was degassed, refilled with hydrogen gas, and stirred overnight at room temperature. The mixture was filtered through Celite and washed with methanol. The combined filtrate was collected and concentrated under vacuum to afford the crude product, which was used directly in the next step without further purification (370 mg, 97% yield). LCMS m/z 462.58 (M+H)⁺.

### Step I: 5-(3-aminopropyl)-2-methoxy-4-(4-(4-methylpiperazin-1-yl)piperidin-1-yl)aniline

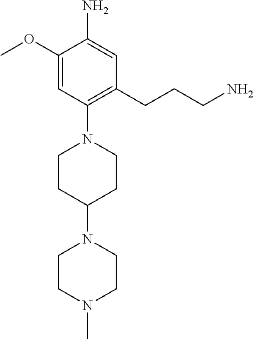 [SMILES: COc1cc(N2CCC(N3CCN(C)CC3)CC2)c(CCCN)cc1N]

To a solution of tert-butyl (3-(5-amino-4-methoxy-2-(4-(4-methylpiperazin-1-yl)piperidin-1-yl)phenyl)propyl)carbamate (80 mg, 0.17 mmol) in dioxane (3 ml) was hydrochloride in dioxane (4 N, 3 ml, 12 mmol). The mixture was stirred at room temperature overnight. Solvent was removed under reduced pressure, and the residue was used directly in the next step without further purification. LCMS m/z 362.44 (M+H)⁺.

### Step J: N-(3-(5-amino-4-methoxy-2-(4-(4-methylpiperazin-1-yl)piperidin-1-yl)phenyl)propyl)-3-(4-((5-bromo-2-chloropyrimidin-4-yl)amino)-3-(N-methylmethylsulfonamido)phenyl)propanamide

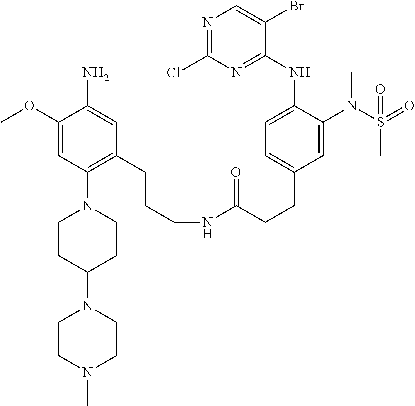 [SMILES: COc1cc(N2CCC(N3CCN(C)CC3)CC2)c(CCCNC(=O)CCc2ccc(Nc3nc(Cl)ncc3Br)c(N(C)S(C)(=O)=O)c2)cc1N]

To a solution of 3-(4-((5-bromo-2-chloropyrimidin-4-yl)amino)-3-(N-methylmethylsulfonamido)phenyl)propanoic acid (69 mg. 0.15 mmol) in dry DMF (2 ml) was added 1-Ethyl-3-(3-dimethylaminopropyl)carbodiimide hydrochloride (40 mg, 0.2 mmol), N-hydroxysuccinimide (23 mg, 0.2 mmol) and diisopropylethyalamine (100 mg, 0.75 mmol). The mixture was stirred at room temperature overnight. 5-(3-aminopropyl)-2-methoxy-4-(4-(4-methylpiperazin-1-yl)piperidin-1-yl)aniline was added and the mixture was stirred room temperature overnight. Water (10 ml) was added and the mixture was extracted with dichloromethane. The crude product was purified by column chromatography on silica gel using dichloromethane/methanol (90/10) to afford the product (55 mg, 45% yield). LCMS m/z 808.77 (M+H)⁺.

### Step K: N-(3⁵-bromo-1⁶-methoxy-1⁴-(4-(4-methylpiperazin-1-yl)piperidin-1-yl)-8-oxo-2,4,9-triaza-3(2,4)-pyrimidina-1(1,3),5(1,4)-dibenzenacyclododecaphane-5²-yl)-N-methylmethanesulfonamide

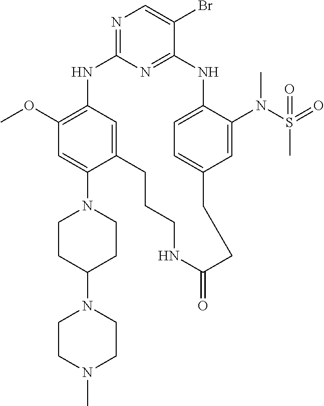 [SMILES: COc1cc(N2CCC(N3CCN(C)CC3)CC2)c2cc1Nc1ncc(Br)c(n1)Nc1ccc(cc1N(C)S(C)(=O)=O)CCC(=O)NCCC2]

To a solution of N-(3-(5-amino-4-methoxy-2-(4-(4-methylpiperazin-1-yl)piperidin-1-yl)phenyl)propyl)-3-(4-((5-bromo-2-chloropyrimidin-4-yl)amino)-3-(N-methylmethylsulfonamido)phenyl)propanamide (55 mg, 0.07 mmol) in 2-methoxyethanol (5 ml) was added methanesulfonic acid (20 mg, 0.2 mmol). The mixture was stirred at 90° C. overnight. Saturated NaHCO₃ aqueous solution (20 mL) was added to quench the reaction, and the product was extracted with chloroform/isopropanol=3:1 and purified by column chromatography on silica gel using MeOH/dichloromethane (10/90) (13 mg, 24% yield).

Other compounds have been prepared analogously. The analytical data for some of the synthesized compounds are shown in Table 3 below.

TABLE 3
Compounds and Analytical Data

| ID | MS [M + H]⁺ m/z | NMR |
|---|---|---|
| A1 | 559.39 | |
| A2 | 588.35 | 1H NMR (300 MHz, DMSO-d6) δ 8.49 (s, 1H), 8.05 (s, 1H), 7.41 |
| | | (s, 1H), 7.36 (d, J = 2.6 Hz, 1H), 7.28 (d, J = 8.6 Hz, 1H), 7.19 |
| | | (dd, J = 8.6, 2.6 Hz, 1H), 6.89 (s, 1H), 6.71 (s, 1H), 4.50-4.24 |
| | | (m, 2H), 3.81 (s, 3H), 2.99 (s, 3H), 2.96 (s, , 3H), 2.77-2.70 (m, |
| | | 4H), 2.50-2.39 (m, 4H), 2.36-2.23 (m, 2H), 2.24 (s, 3H), 1.83-1.66 (m, 2H). |
| A3 | 616.75 | 1H NMR (300 MHz, DMSO-d6) δ 8.29 (s, 1H), 8.09 (s, 1H), 7.45 |
| | | (s, 2H), 7.33 (d, J = 8.8 Hz, 1H), 7.27 (d, J = 2.7 Hz, 1H), 7.08 |
| | | (dd, J = 8.8, 2.7 Hz, 1H), 6.75 (s, 1H), 4.35 (t, J = 5.3 Hz, 2H), |
| | | 3.81 (s, 3H), 3.08 (s, 3H), 2.99 (s, 3H), 2.75 (t, J = 4.7 Hz, 4H), |
| | | 2.61-2.38 (m, 4H), 2.27 (s, 3H), 2.13-2.02 (m, 2H), 1.83- |
| | | 1.71 (m, 2H), 1.59-1.45 (m, 2H), 1.30-1.19 (m, 2H). |
| A4 | 602.53 | |
| A5 | 618.52 | |
| A6 | 573.38 | |
| A7 | 590.51 | |
| A8 | 630.58 | 1H NMR (300 MHz, DMSO-d₆) δ 8.38 (s, 1H), 8.09 (s, 1H), 7.44 |
| | | (d, J = 2.8 Hz, 2H), 7.32 (d, J = 8.8 Hz, 1H), 7.23 (d, J = 2.8 Hz, |
| | | 1H), 7.08 (dd, J = 8.9, 2.7 Hz, 1H), 6.75 (s, 1H), 4.38-4.31 (m, |
| | | 2H), 3.81 (s, 3H), 3.16 (q, J = 7.6 Hz, 2H), 3.10 (s, 3H), 2.77- |
| | | 2.69 (m, 4H), 2.48-2.34 (m, 4H), 2.22 (s, 3H), 2.14-2.03 (m, |
| | | 2H), 1.84-1.72 (m, 2H), 1.56-1.45 (m, 2H), 1.32-1.19 (m, |
| | | 2H), 1.16 (t, J = 7.3 Hz, 3H). |
| A9 | 699.65 | 1H NMR (500 MHz, DMSO-d6) δ 8.26 (s, 1H), 8.08 (s, 1H), 7.43 |
| | | (d, J = 1.9 Hz, 2H), 7.33 (d, J = 8.9 Hz, 1H), 7.25 (d, J = 2.8 Hz, |
| | | 1H), 7.08 (dd, J = 8.8, 2.8 Hz, 1H), 6.72 (s, 1H), 4.34 (t, J = 5.6 |
| | | Hz, 2H), 3.79 (s, 3H), 3.07 (s, 3H), 2.98 (s, 3H), 2.82 (d, J = 10.8 |
| | | Hz, 2H), 2.67-2.54 (m, 2H), 2.55-2.37 (m, 4H), 2.37-2.17 |
| | | (m, 5H), 2.13 (s, 3H), 2.10-2.03 (m, 2H), 1.88-1.74 (m, 4H), |
| | | 1.59-1.38 (m, 4H), 1.23 (q, J = 8.7 Hz, 2H). |
| A10 | 630.54 | 1H NMR (300 MHz, DMSO-d6) δ 8.13 (s, 1H), 7.95 (s, 1H), 7.48 |
| | | (d, J = 9.1 Hz, 2H), 7.40 (d, J = 8.9 Hz, 1H), 7.31 (d, J = 2.7 Hz, |
| | | 1H), 7.12 (d, J = 9.6 Hz, 1H), 6.77 (s, 1H), 4.40-4.33 (m, 2H), |
| | | 3.82 (s, 3H), 3.64 (q, J = 7.2 Hz, 2H), 3.06 (s, 3H), 2.78-2.68 |
| | | (m, 4H), 2.48-2.36 (m, 4H), 2.21 (s, 3H), 2.17-2.02 (m, 2H), |
| | | 1.81-1.69 (m, 2H), 1.56-1.42 (m, 2H), 1.32-1.19 (m, 2H), |
| | | 0.93-0.81 (m, 3H). |
| A11 | 600.57 | 1H NMR (300 MHz, DMSO-d6) δ 8.75 (s, 1H), 8.03 (d, J = 3.8 |
| | | Hz, 1H), 7.47 (s, 1H), 7.38-7.19 (m, 3H), 7.09 (dd, J = 8.7, 2.8 |
| | | Hz, 1H), 6.73 (s, 1H), 4.38-4.31 (m, 2H), 3.81 (s, 3H), 3.04 (s, |
| | | 3H), 2.96 (s, 3H), 2.76-2.69 (m, 4H), 2.47-2.36 (m, 4H), 2.22 |
| | | (s, 3H), 2.13-2.04 (m, 2H), 1.81-1.72 (m, 2H), 1.56-1.46 (m, |
| | | 2H), 1.29-1.21 (m, 2H). |
| A12 | 662.55 | |
| A13 | 650.65 | |
| A14 | 630.61 | 1H NMR (300 MHz, DMSO-d₆) δ 8.22 (s, 1H), 8.10 (s, 1H), 7.55- |
| | | 7.45 (m, 3H), 7.28 (d, J = 2.7 Hz, 1H), 7.08 (dd, J = 8.8, 2.8 Hz, |
| | | 1H), 6.78 (s, 1H), 4.38-4.31 (m, 2H), 3.83 (s, 3H), 3.11 (s, 3H), |
| | | 3.02 (s, 3H), 2.79-2.71 (m, 4H), 2.50-2.34 (m, 4H), 2.26- |
| | | 2.16 (m, 5H), 1.73-1.61 (m, 2H), 1.61-1.46 (m, 4H), 1.22- |
| | | 1.10 (m, 2H). |
| A15 | 713.76 | 1H NMR (300 MHz, DMSO-d₆) δ 8.22 (s, 1 H), 8.10 (s, 1 H), 7.55- |
| | | 7.46 (m, 3H), 7.28 (d, J = 2.7 Hz, 1H), 7.08 (dd, J = 8.8, 2.7 Hz, |
| | | 1H), 6.76 (s, 1H), 4.35 (s, 2H), 3.81 (s, 3H), 3.11 (s, 3H), 3.02 (s, |
| | | 3H), 2.90-2.80 (m, 2H), 2.75-2.54 (m, 4H), 2.48-2.30 (m, |
| | | 4H), 2.30-2.25 (m, 1H), 2.24-2.16 (m, 5H), 1.89-1.79 (m, |
| | | 2H), 1.70-1.60 (m, 2H), 1.58-1.39 (m, 6H), 1.21-1.10 (m, |
| | | 2H). |
| A16 | 602.43 | |
| A17 | 644.62 | |
| A18 | 585.48 | |
| A19 | 684.61 | 1H NMR (300 MHz, DMSO-d₆) δ 8.77 (s, 1H), 8.11 (s, 1H), 7.64 |
| | | (d, J = 8.7 Hz, 1H), 7.52 (d, J = 12.7 Hz, 1H), 7.37 (s, 1H), 6.76 |
| | | (s, 1H), 4.44-4.34 (m, 2H), 3.80 (s, 3H), 3.17 (s, 3H), 2.84 (d, J = |
| | | 10.9 Hz, 2H), 2.63 (t, J = 11.3 Hz, 2H), 2.52-2.44 (m, 4H), |
| | | 2.37-2.25 (m, 4H), 2.24-2.18 (m, 1H), 2.14 (s, 3H), 1.88- |
| | | 1.77 (m, 2H), 1.73-1.62 (m, 2H), 1.59-1.41 (m, 6H), 1.18- |
| | | 1.06 (m, 2H). |
| A20 | 699.67 | |
| A21 | 630.58 | 1H NMR (300 MHz, DMSO-d₆) δ 8.29 (s, 1H), 8.09 (s, 1H), 7.45 |
| | | (d, J = 8.0 Hz, 2H), 7.34 (d, J = 8.8 Hz, 1H), 7.26 (d, J = 2.7 Hz, |
| | | 1H), 7.10 (dd, J = 8.8, 2.7 Hz, 1H), 6.74 (s, 1H), 4.39-4.31 (m, |
| | | 2H), 4.06 (q, J = 6.8 Hz, 2H), 3.07 (s, 3H), 2.98 (s, 3H), 2.76- |
| | | 2.66 (m, 4H), 2.46-2.32 (m, 4H), 2.21 (s, 3H), 2.12-2.02 (m, |
| | | 2H), 1.83-1.71 (m, 2H), 1.58-1.45 (m, 2H), 1.35 (t, J = 6.9 Hz, |
| | | 3H), 1.30-1.19 (m, 2H). |
| A22 | 644.64 | 1H NMR (300 MHz, DMSO-d₆) δ 8.30 (s, 1H), 8.09 (s, 1H), 7.48 |
| | | (s, 1H), 7.42 (s, 1H), 7.35 (d, J = 8.8 Hz, 1H), 7.27 (d, J = 2.7 Hz, |
| | | 1H), 7.10 (dd, J = 8.8, 2.8 Hz, 1H), 6.76 (s, 1H), 4.59 (p, J = 6.0 |
| | | Hz, 1H), 4.40-4.33 (m, 2H), 3.08 (s, 3H), 2.99 (s, 3H), 2.76- |
| | | 2.66 (m, 4H), 2.46-2.35 (m, 4H), 2.22 (s, 3H), 2.15-1.97 (m, |
| | | 2H), 1.83-1.71 (m, 2H), 1.58-1.47 (m, 2H), 1.28 (d, J = 6.0 |
| | | Hz, 6H), 1.26-1.19 (m, 2H). |
| A23 | 644.6 | 1H NMR (300 MHz, DMSO-d₆) δ 8.12 (s, 1H), 7.98 (s, 1H), 7.57- |
| | | 7.48 (m, 3H), 7.31 (d, J = 2.8 Hz, 1H), 7.09 (dd, J = 8.8, 2.7 Hz, |
| | | 1H), 6.78 (s, 1H), 4.45-4.28 (m, 2H), 3.83 (s, 3H), 3.59 (q, J = |
| | | 7.1 Hz, 2H), 3.08 (s, 3H), 2.78-2.70 (m, 4H), 2.48-2.34 (m, |
| | | 4H), 2.26-2.14 (m, 5H), 1.70-1.45 (m, 4H), 1.20-1.05 (m, |
| | | 4H), 0.78 (t, J = 7.1 Hz, 3H). |
| A24 | 644.62 | 1H NMR (300 MHz, DMSO-d₆) δ 8.28 (s, 1H), 8.09 (s, 1H), 7.44 |
| | | (s, 2H), 7.33 (d, J = 8.8 Hz, 1H), 7.26 (d, J = 2.7 Hz, 1H), 7.09 |
| | | (dd, J = 8.8, 2.8 Hz, 1H), 6.73 (s, 1H), 4.39-4.31 (m, 2H), 3.80 |
| | | (s, 3H), 3.07 (s, 3H), 2.98 (s, 3H), 2.84 (d, J = 11.3 Hz, 2H), 2.61 |
| | | (t, J = 11.2 Hz, 2H), 2.19 (s, 6H), 2.14-2.03 (m, 3H), 1.85- |
| | | 1.72 (m, 4H), 1.56-1.39 (m, 4H), 1.31-1.25 (m, 2H). |
| A25 | 594.64 | |
| A26 | 698.75 | 1H NMR (300 MHz, DMSO-d₆) δ 8.55 (s, 1H), 8.18 (s, 1H), 7.64- |
| | | 7.52 (m, 2H), 7.48 (dd, J = 8.9, 2.9 Hz, 1H), 7.44-7.36 (m, |
| | | 2H), 6.76 (s, 1H), 4.45-4.34 (m, 2H), 3.81 (s, 3H), 2.84 (d, J = |
| | | 11.1 Hz, 2H), 2.63 (t, J = 11.3 Hz, 2H), 2.51-2.43 (m, 4H), 2.38- |
| | | 2.24 (m, 4H), 2.24-2.18 (m, 1 H), 2.14 (s, 3H), 2.10-2.01 |
| | | (m, 2H), 1.88-1.69 (m, 4H), 1.88-1.69 (m, 4H), 1.53-1.38 |
| | | (m, 4H), 1.34-1.19 (m, 3H), 1.14 (d, J = 6.8 Hz, 6H). |
| A27 | 630.54 | |
| A28 | 642.59 | |
| A29 | 743.6 | 1H NMR (500 MHz, DMSO-d₆) δ 8.15 (s, 1H), 8.05 (s, 1H), 7.43 |
| | | (d, J = 9.3 Hz, 2H), 7.34 (d, J = 8.8 Hz, 1H), 7.27 (d, J = 2.8 Hz, |
| | | 1H), 7.07 (dd, J = 8.8, 2.8 Hz, 1H), 6.73 (s, 1H), 4.35 (t, J = 5.6 |
| | | Hz, 2H), 3.79 (s, 3H), 3.08 (s, 3H), 2.99 (s, 3H), 2.82 (d, J = 11.2 |
| | | Hz, 2H), 2.67-2.56 (m, 2H), 2.40-2.25 (m, 4H), 2.25-2.18 |
| | | (m, 1H), 2.15 (s, 3H), 2.12-2.02 (m, 2H), 1.84-1.73 (m, 4H), |
| | | 1.56-1.38 (m, 4H), 1.31-1.20 (m, 2H). |
| A30 | 759.56 | |
| A31 | 644.65 | 1H NMR (300 MHz, DMSO-d₆) δ 8.30 (s, 1H), 8.09 (s, 1H), 7.46 |
| | | (d, J = 5.0 Hz, 2H), 7.34 (d, J = 8.8 Hz, 1H), 7.27 (d, J = 2.8 Hz, |
| | | 1H), 7.10 (dd, J = 8.8, 2.7 Hz, 1H), 6.78 (s, 1H), 4.41-4.32 (m, |
| | | 2H), 3.81 (s, 3H), 3.56-3.46 (m, 4H), 3.08 (s, 3H), 2.99 (s, 3H), |
| | | 2.77-2.64 (m, 4H), 2.16-2.06 (m, 2H), 2.03 (s, 3H), 1.83- |
| | | 1.72 (m, 2H), 1.60-1.48 (m, 2H), 1.35-1.19 (m, 2H). |
| A32 | 686.66 | 1H NMR (300 MHz, DMSO-d₆) δ 8.28 (s, 1H), 8.09 (s, 1H), 7.44 |
| | | (s, 2H), 7.34 (d, J = 8.8 Hz, 1H), 7.26 (d, J = 2.7 Hz, 1H), 7.09 |
| | | (dd, J = 8.8, 2.8 Hz, 1H), 6.74 (s, 1H), 4.40-4.31 (m, 2H), 3.80 |
| | | (s, 3H), 3.62-3.55 (m, 4H), 3.07 (s, 3H), 2.98 (s, 3H), 2.84 (d, J = |
| | | 11.1 Hz, 2H), 2.63 (t, J = 11.3 Hz, 2H), 2.51-2.43 (m, 4H), |
| | | 2.23-2.12 (m, 1H), 2.12-2.02 (m, 2H), 1.90-1.72 (m, 4H), |
| | | 1.56-1.41 (m, 4H), 1.32-1.19 (m, 2H). |
| A33 | 712.81 | 1H NMR (300 MHz, DMSO-d₆) δ 9.31 (s, 1H), 8.19 (s, 1H), 7.54- |
| | | 7.34 (m, 4H), 7.29-7.20 (m, 1H), 6.75 (s, 1H), 4.39-4.31 (m, |
| | | 2H), 3.81 (s, 3H), 2.84 (d, J = 11.3 Hz, 2H), 2.63 (t, J = 11.4 Hz, |
| | | 2H), 2.57-2.47 (m, 4H), 2.45-2.34 (m, 4H), 2.31-2.23 (m, |
| | | 1H), 2.20 (s, 3H), 2.11-2.03 (m, 2H), 1.87-1.70 (m, 4H), 1.66 |
| | | (d, J = 13.6 Hz, 6H), 1.54-1.40 (m, 4H), 1.28-1.22 (m, 2H). |
| A34 | 726.79 | |
| A35 | 615.41 | |
| A36 | 745.86 | |
| A37 | 771.86 | |
| A38 | 700.49 | |
| A39 | 714.63 | |
| A40 | 728.79 | |
| A41 | 771.80 | ¹H NMR (300 MHz, DMSO-d₆) δ 8.29 (s, 1H), 8.26 (s, 1H), 8.05 |
| | | (d, J = 8.3 Hz, 1H), 7.90 (t, J = 6.2 Hz, 1 H), 7.85 (s, 1H), 7.69 (s, |
| | | 1H), 7.57 (d, J = 1.9 Hz, 1H), 7.20 (dd, J = 8.4, 1.9 Hz, 1H), 6.83 |
| | | (s, 1H), 3.85 (s, 3H), 3.24 (s, 3H), 3.20-3.12 (m, 5H), 2.99- |
| | | 2.89 (m, 4H), 2.69 (t, J = 11.4 Hz, 2H), 2.62-2.22 (m, 13H), |
| | | 2.17 (s, 3H), 1.93-1.83 (m, 2H), 1.62-1.38 (m, 4H). |
| A42 | 772.53 | |

### Example B: In Vitro Biochemical Kinase Assay

The biochemical kinase assay was performed at Reaction Biology Corporation (www.reactionbiology.com, Malvern, Pa.) following procedures described in the reference (Anastassiadis, T, et al, Nature Biotechnology, 2011, 29, 1039). Base Reaction buffer includes 20 mM Hepes (pH 7.5), 10 mM MgCl₂, 1 mM EGTA, 0.02% Brij35, 0.02 mg/ml BSA, 0.1 mM Na₃VO₄, 2 mM DTT and 1% DMSO. Required cofactors are added individually to each kinase reaction. Testing compounds were dissolved in 100% DMSO to specific concentration. The serial dilution was conducted by Integra Viaflo Assist in DMSO. Compounds in 100% DMSO are into the kinase reaction mixture by Acoustic technology (Echo550; nanoliter range), incubate for 20 min at room temperature, followed by ³³P-ATP and incubation for 2 hours at room temperature. Radioactivity was then detected by filter-binding method. Kinase activity data were expressed as the percent remaining kinase activity in test samples compared to vehicle (dimethyl sulfoxide) reactions. IC₅₀ values and curve fits were obtained using Prism (GraphPad Software).

The testing data (IC₅₀ in nM) for some of the compounds are shown in Table 4.

### Example C: In Vitro Anti-Proliferation Assay

The BaF3 cell proliferation assay was performed at Pharmaron (www.pharmaron.com, Beijing, China). The Ba/F3_WT and Ba/F3_Del19/T790M/C797S cell lines were maintained in 1640 medium containing 10% FBS, 1*PS and 1*Glutamax. Only cells with viability greater than 90% are used for assays. Dispense 30 μl of cell suspension at 700/well into 384 well microplate, add 30 nl compound solution into 384 cell plate by Echo, cells were incubated for 72 hr in 37° C./5% CO₂ incubator, add 30 μl reagent CelltiterGlo assay kit (CTG, Promega) per well and shake plates, incubate the plate (avoiding light) at 37° C./5% CO₂ for 30 min. and read the luminescence by Envision. The luminescence values were converted to % Inhibition. Calculate IC₅₀ by fitting % inhibition values and log of compound concentrations to nonlinear regression (dose response−variable slope) with Graphpad 5.0.

Y=Bottom+(Top−Bottom)/(1+10\hat{ }((Log IC₅₀−X)*HillSlope))

X: log of inhibitor concentration; Y: % Inhibition.

Data of some tested compounds are shown in Table 4.

TABLE 4
Testing results

| | | | IC50 (nM, | |
|---|---|---|---|---|
| | IC50 (nM, | | Ba/F3_EGFR_del19/ | IC50 (nM, |
| | EGFR_L858R/ | IC50 (nM, | T790M/C797S cell | Ba/F3_WT cell |
| Compound | T790M/C797S) | EGFR_WT) | proliferation) | proliferation,) |
| A1 | 5582 | | | |
| A2 | 643.4 | | | |
| A3 | 9.855 | | 135 | 1177 |
| A4 | 46.5 | | 252 | 3321 |
| A5 | 27.9 | | | |
| A6 | 10000 | | | |
| A7 | 3755 | | | |
| A8 | 2.7 | | 538 | 3429 |
| A9 | 0.5 | 5.9 | 34 | 1387 |
| A10 | 0.5 | | 125 | 1836 |
| A11 | 17 | | | |
| A12 | 0.6 | | | |
| A13 | 2 | | 108 | 3766 |
| A14 | 16.7 | | 897 | 2958 |
| A15 | 0.5 | | 195 | 2663 |
| A16 | 20.3 | | | |
| A17 | 82.2 | | | |
| A18 | 145.2 | | 520 | >10000 |
| A19 | 179.5 | | | |
| A20 | 2.6 | | 89 | 1451 |
| A21 | 40.5 | | | |
| A22 | 503.5 | | | |
| A23 | 2.6 | | 157 | 2662 |
| A24 | 2.7 | 10.8 | 63 | 3332 |
| A25 | 2505 | | | |
| A26 | 249.5 | | 1111 | 1793 |
| A27 | 2.3 | | 329.5 | 3396 |
| A28 | 1.4 | | 194.2 | 2974 |
| A29 | 0.5 | 9.2 | 16.4 | 1322 |
| A30 | 0.6 | 14.9 | 22.4 | 1692 |
| A31 | 12.7 | | 153.7 | >10000 |
| A32 | 0.6 | | 105.7 | 4839 |
| A33 | 1.8 | 413.1 | 75 | 6486 |
| A34 | 2 | | 141.6 | 3816 |
| A35 | 289 | | 1045 | 2242 |
| A36 | 2.9 | | | |
| A37 | 0.6 | | | |
| A38 | 0.3 | | | |
| A39 | 7.1 | | | |
| A40 | 4.3 | | | |
| A41 | 0.4 | | | |
| A42 | 3.8 | | | |

### Example D: In Vivo Studies Using Cell Transplant Xenograft (CTX) Model

The pharmacological experiments in vivo were performed on BALB/c nude mice that subcutaneously implanted BaF3-EGFR-Del19/T790M/C797S cells. BALB/c nude mice, female, 6-8 weeks, weighted about 16-19 grams, the mice were kept in a special pathogen-free environment, and in a single ventilation cage (3 mice per cage). The bedding and water of all the cages were disinfected before use. All animals were free to obtain standard certified commercial laboratory diets. The BaF3-EGFR-Del19/T790M/C797S cell (5×10⁶ cells/mice) was implanted subcutaneously for tumor growth. After 10 days, the experiment was started the average tumor volume reached about 130 mm³. The mice were divided into 4 groups with 6 mice in each group. Compound A29 was orally administered once daily continuously for 15 days at 5 mg/kg, 15 mg/kg and 45 mg/kg. The tumor volume was measured twice a week with a two-dimensional caliper and the volume was measured in cubic millimeters and calculated by the following formula: V=0.5 a×b2, where a and b were the major and minor diameters of the tumor, respectively. The tumor volume at day n is expressed as Relative Tumor Volume (RTV) and calculated according to the following formula: RTV=TVₙ/TV₀, where TVₙ is the tumor volume at day n and TV₀ is the tumor volume at day 0. The T/C (the ratio of tumor volume in control versus treated mice) is determined by TIC (%)=(mean RTV of treated group)/(mean RTV of control group)×100%. The tumor growth inhibition rate TGI_{TV} (%) is calculated using the formula TGI_{TV} (%)=(1−T/C)×100%. The data is shown in Table 5 and FIG. 1.

TABLE 5
Tumor Volume (mm³) over the time after administration of compounds

| | | Tumor Volume (mm³) | | | | | | |
|---|---|---|---|---|---|---|---|---|
| Compound | Dose | Day 0 | Day 2 | Day 4 | Day 7 | Day 9 | Day 11 | Day 14 |
| blank | N/A | 130 | 276 | 430 | 933 | 1381 | 2044 | 3265 |
| A29 | 5 mg/kg | 130 | 212 | 279 | 462 | 633 | 819 | 1061 |
| | 15 mg/kg | 130 | 171 | 226 | 346 | 469 | 597 | 781 |
| | 45 mg/kg | 130 | 147 | 172 | 108 | 78 | 25 | 0 |

Compound A29 showed significant tumor growth inhibition at all doses. At the end of the experiment, for 5 mg/kg, 15 mg·kg and 45 mg/kg dose, the TGI_{TV} (%) value is 68%, 76%, and 100% respectively.

Unless otherwise indicated, all numbers expressing quantities of ingredients, properties such as molecular weight, reaction conditions, and etc. used in herein are to be understood as being modified in all instances by the term “about.” Each numerical parameter should at least be construed in light of the number of reported significant digits and by applying ordinary rounding techniques. Accordingly, unless indicated to the contrary, the numerical parameters may be modified according to the desired properties sought to be achieved, and should, therefore, be considered as part of the disclosure. At the very least, the examples shown herein are for illustration only, not as an attempt to limit the scope of the disclosure.

The terms “a,” “an,” “the” and similar referents used in the context of describing embodiments of the present disclosure (especially in the context of the following claims) are to be construed to cover both the singular and the plural, unless otherwise indicated herein or clearly contradicted by context. All methods described herein may be performed in any suitable order unless otherwise indicated herein or otherwise clearly contradicted by context. The use of any and all examples, or exemplary language (e.g., “such as”) provided herein is intended merely to better illustrate embodiments of the present disclosure and does not pose a limitation on the scope of any claim. No language in the specification should be construed as indicating any non-claimed element essential to the practice of the embodiments of the present disclosure.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability.

Certain embodiments are described herein, including the best mode known to the inventors for carrying out the embodiments. Of course, variations on these described embodiments will become apparent to those of ordinary skill in the art upon reading the foregoing description. The inventor expects skilled artisans to employ such variations as appropriate, and the inventors intend for the embodiments of the present disclosure to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

1. A compound represented by a Formula: 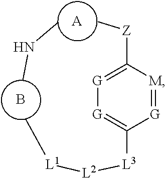
or a pharmaceutically acceptable salt thereof;
wherein 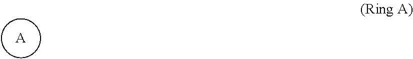
is an optionally substituted 6-membered aromatic heterocyclic ring or an optionally substituted 9-membered fused aromatic bicyclic heterocylic ring; 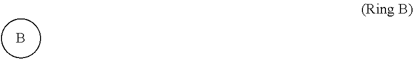
is an optionally substituted 5- or 6-membered aromatic ring, or an optionally substituted 10- or 13-membered fused bicyclic ring containing one 5- or 6-membered aromatic ring and one 5, 6, or 7-membered saturated ring;
Z is O, S(O)₀₋₂, CR^{A1} R^{B1}, or NR^{A};
M is CR¹ or N;
each G is independently CR or N;
L¹ and L³ are independently a covalent bond, O, NR^{A}, S(O)₀₋₂, CR^{A1}R^{B1}, CR^{A1}═CR^{B1}, —C(O)NR^{A}—, —NR^{A}(CO)—, S(O)₁₋₂NR^{A}, or NR^{A}C(O)NR^{B};
L² is an optionally substituted C₁₋₁₂ alkylene, Cₘ alkylene-C(O)NR^{A}—Cₙ alkylene, Cₘ alkylene-NR^{A}(CO)—Cₙ alkylene, Cₘ alkylene-CR^{A1}═CR^{B1}—Cₙ alkylene, or Cₘ alkylene-O—Cₙ alkylene, wherein m is 1 to 12, n is 1 to 12, provided that the sum of m and n is no more than 12, wherein L² has, as chemically appropriate, 0, 1, 2, 3, 4, 5, 6, 7, 8, 9, 10, 11, or 12 substituents, and the substituents of L² are independently F, Cl, Br, I, OH, ═O, C₁₋₆ alkyl, or C₁₋₆ cycloalkyl;
each R^{A1} and each R^{B1} are independently H, F, Cl, Br, I, or C₁₋₆ hydrocarbyl;
each R is independently H, F, Cl, Br, I, —NR^{A}R^{B}, C₁₋₆ hydrocarbyl, —OH, —CN, —NO₂, —O—C₁₋₆ alkyl, or —C(O)O—C₁₋₆ alkyl;
R¹ is H, F, Cl, Br, I, —NR^{A}R^{B}, C₁₋₆ hydrocarbyl, —OH, —CN, —NO₂, —O—C₁₋₆ alkyl, —C(O)O—C₁₋₆ alkyl, —S(O)₁₋₂R^{A}; —P(O)R^{A}R^{B}, —NR^{A}S(O)₂R^{B}, —S(O)₂NR^{A}R^{B}, —C(O)NR^{A}R^{B}, —NR^{A}C(O)R^{A}R^{B};
each R^{A} and each R^{B} are independently H or C₁₋₆ hydrocarbyl; and
wherein each R^{A}, each R^{B}, each R^{A1}, each R^{B1}, each R, and each R¹ are independently optionally halogenated.

2. The compound of claim 1, wherein Ring A is: 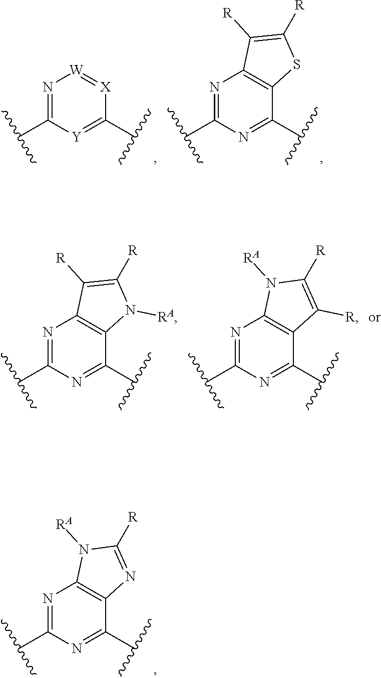
wherein each right side of the above structures is directly attached to Z in the Formula of claim 1;
W is N or CR²;
X is N or CR³;
Y is N or CR⁴; and
Ring B is: 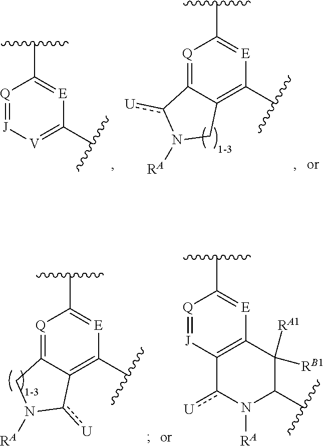
wherein L¹-L² kin the Formula of claim 1 is an optionally substituted C₁₋₈ alkylene and L³ is O;
wherein each top side of the above structures is linked to Ring A via NH in the Formula of claim 1;
each of the above structures of Ring A and Ring B is optionally substituted;
each E is independently CR, NR^{A}, O, or S;
each Q is independently CR⁵, NR^{A}, O, or S;
each J is independently a bond, CR⁶, or N;
U is O or H₂;
V is CR², NR^{A}, O, or S;
R² and R³ are independently H, F, Cl, Br, I, —NR^{A}R^{B}, C₁₋₆ hydrocarbyl, —OH, —CN, —NO₂, —O—C₁₋₆ alkyl, —C(O)O—C₁₋₆ alkyl, or —NR^{A}C(O)O—C₁₋₆ alkyl;
R⁴, R⁵, and R⁶ are independently H, F, Cl, Br, I, —NR^{A}R^{B}, C₁₋₆ hydrocarbyl, —OH, —CN, —NO₂, —O—C₁₋₆ alkyl, or —C(O)O—C₁₋₆ alkyl;
R² and R³ may connect and together with Ring A to form a fused ring system;
R⁵ and R⁶ may connect and together with Ring B to form a fused ring system;
R⁷ is H, F, Cl, Br, I, NR^{A}R^{B}, NR^{A} (CR^{A1}R^{B1})₁₋₃—NR^{A}R^{B}, C₁₋₆ hydrocarbyl, —OH, —CN, —NO₂, —O—C₁₋₆ alkyl, or —C(O)O—C₁₋₆ alkyl, —NR^{A}S(O)₂R^{B}, —S(O)₂NR^{A}R^{B}, —C(O)NR^{A}R^{B}, —NR^{A}C(O)R^{A}R^{B}, —NR^{A}C(O)NR^{A}R^{B}, OC(O)NR^{A}R^{B}, CR^{A1}R^{B1}C(O)NR^{A}R^{B}, an optionally substituted 5- or 6-membered saturated mono-cyclic ring containing 1 or 2 ring N atoms and 0 to 1 ring O atom, or an optionally substituted 8 to 12 membered saturated bicyclic ring system containing 2 to 3 ring N atoms and 0 to 1 ring O atom; and
wherein each R², each R³, each R⁴, each R⁵, each R⁶, and each R⁷ are independently optionally halogenated.

3. The compound of claim 1, wherein Ring A is: 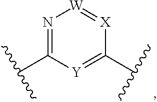
and
wherein the Ring A-Z is:

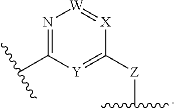

4. The compound of claim 1, wherein Ring A is an optionally substituted pyrimidin-2,4-di-yl, an optionally substituted pyrimidin-4,6-di-yl, an optionally substituted pyridazin-4,6-di-yl, an optionally substituted pyridin-2,4-di-yl, an optionally substituted 1,3,5-triazin-2,4-di-yl, an optionally substituted 1,2,4-triazin-3,5-di-yl, an optionally substituted 1,2,3-triazin-4,6-di-yl, 1,2,3,5-tetrazin-4,6-di-yl, or an optionally substituted pyridazin-4,6-di-yl.

5. (canceled)

6. (canceled)

7. (canceled)

8. The compound of claim 1, wherein Ring A is:

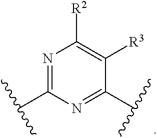

9. The compound of claim 1, wherein Ring B is:

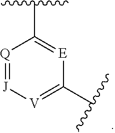

10. (canceled)

11. (canceled)

12. (canceled)

13. (canceled)

14. The compound of claim 1, wherein Ring B is 1,3-benzen-di-yl.

15. The compound of claim 1, wherein Z is NR^{A}.

16. The compound of claim 15, wherein R^{A} is H.

17. The compound of claim 1, wherein 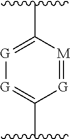
is 1,2,4,5-tetrazin-3,6-di-yl, an optionally substituted 1,2,4-triazin-3,6-di-yl, an optionally substituted pyridazin-3,6-di-yl, an optionally substituted pyrimidin-2,5-di-yl, an optionally substituted pyrazin-2,5-di-yl, an optionally substituted pyridin-2,5-di-yl, or an optionally substituted 1,4-benzen-di-yl.

18. (canceled)

19. (canceled)

20. The compound of claim 1, which is further represented by a Formula: 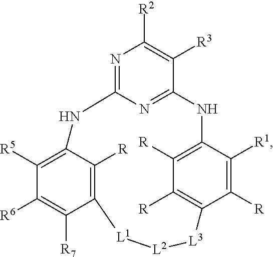
or a pharmaceutically acceptable salt thereof.

21. The compound of claim 1, wherein L¹ is a covalent bond or —NR^{A}C(O)—.

22. (canceled)

23. (canceled)

24. The compound of claim 1, wherein L³ is O or a covalent bond.

25. (canceled)

26. The compound of claim 1, wherein L² is an optionally substituted C₁₋₆ alkylene, Cₘ alkylene-C(O)NR^{A}—Cₙ alkylene, or Cₘ alkylene-NR^{A}(CO)—Cₙ alkylene, wherein m is 1 to 6, n is 1 to 6, provided that the sum of m and n is no more than 6.

27. (canceled)

28. The compound of claim 1, wherein L² is an optionally substituted C₃₋₆ alkylene.

29. (canceled)

30. (canceled)

31. (canceled)

32. The compound of claim 20, wherein R¹ is:

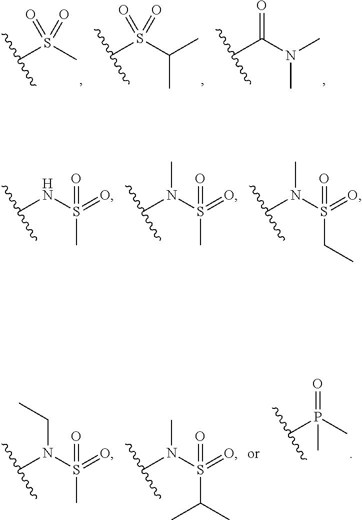

33. (canceled)

34. The compound of claim 20, wherein R³ is F, Cl, Br, or CF₃.

35. The compound of claim 20, wherein R⁵ is —OCH₃, —OCH₂CH₃, or —OC(CH)(CH₃)₂.

36. (canceled)

37. (canceled)

38. The compound of claim 20, wherein R⁷ is an optionally substituted 5- or 6-membered saturated mono-cyclic ring containing 1 or 2 ring N atoms and 0 to 1 ring O atom, or an optionally substituted 8 to 12 membered saturated bicyclic ring system containing 2 to 3 ring N atoms and 0 to 1 ring O atom.

39. The compound of claim 2, wherein R⁷ is: 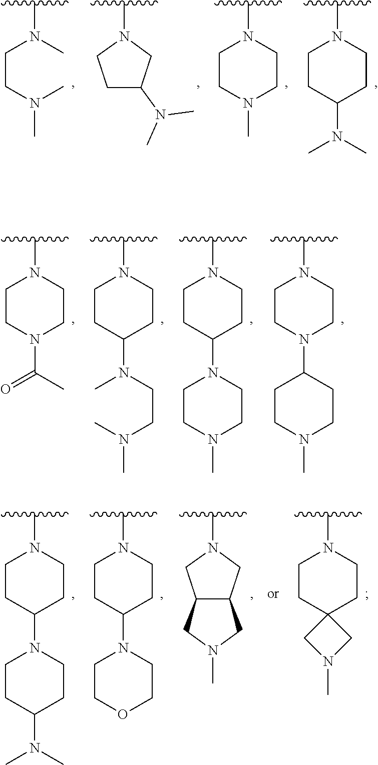
wherein each structure is optionally substituted.

40. The compound of claim 1, which is an optionally substituted 6-oxa-2,4-diaza-3(2,4)-pyrimidina-1(1,3),5(1,4)-dibenzenacyclononaphane, an optionally substituted 6-oxa-2,4-diaza-3(2,4)-pyrimidina-1(1,3),5(1,4)-dibenzenacyclodecaphane, an optionally substituted 6-oxa-2,4-diaza-3(2,4)-pyrimidina-1(1,3),5(1,4)-dibenzenacycloundecaphane, an optionally substituted 6-oxa-2,4-diaza-3(2,4)-pyrimidina-1(1,3),5(1,4)-dibenzenacyclododecaphane, an optionally substituted 6-oxa-2,4,11-triaza-3(2,4)-pyrimidina-1(1,3),5(1,4)-dibenzenacycloundecaphan-10-one, an optionally substituted 6-oxa-2,4,12-triaza-3(2,4)-pyrimidina-1(1,3),5(1,4)-dibenzenacyclododecaphan-11-one, an optionally substituted 2,4,9-triaza-3(2,4)-pyrimidina-1(1,3),5(1,4)-dibenzenacyclododecaphan-8-one, or an optionally substituted 2,4,9-triaza-3(2,4)-pyrimidina-1(1,3),5(1,4)-dibenzenacyclododecaphan-10-one.

41. A compound or a pharmaceutically acceptable salt thereof, wherein the compound is: 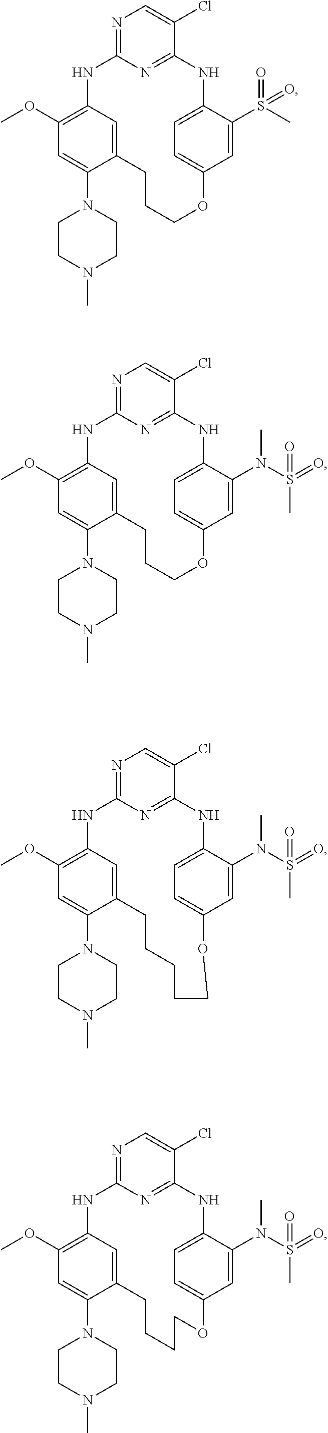 [SMILES: COc1cc(N2CCN(C)CC2)c2cc1Nc1ncc(Cl)c(n1)Nc1ccc(cc1S(C)(=O)=O)OCCC2 ; COc1cc(N2CCN(C)CC2)c2cc1Nc1ncc(Cl)c(n1)Nc1ccc(cc1N(C)S(C)(=O)=O)OCCC2 ; COc1cc(N2CCN(C)CC2)c2cc1Nc1ncc(Cl)c(n1)Nc1ccc(cc1N(C)S(C)(=O)=O)OCCCCC2 ; COc1cc(N2CCN(C)CC2)c2cc1Nc1ncc(Cl)c(n1)Nc1ccc(cc1N(C)S(C)(=O)=O)OCCCC2] 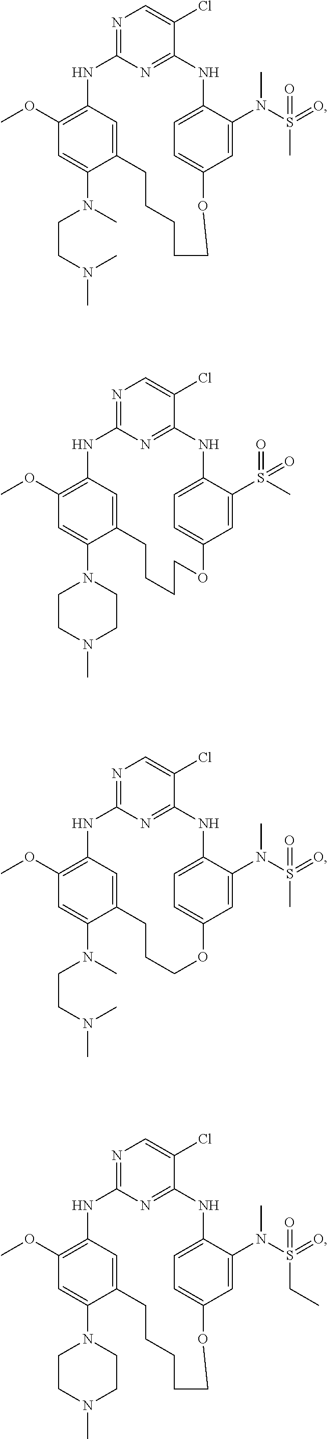 [SMILES: COc1cc(N(C)CCN(C)C)c2cc1Nc1ncc(Cl)c(n1)Nc1ccc(cc1N(C)S(C)(=O)=O)OCCCCC2 ; COc1cc(N2CCN(C)CC2)c2cc1Nc1ncc(Cl)c(n1)Nc1ccc(cc1S(C)(=O)=O)OCCCC2 ; COc1cc(N(C)CCN(C)C)c2cc1Nc1ncc(Cl)c(n1)Nc1ccc(cc1N(C)S(C)(=O)=O)OCCC2 ; CCS(=O)(=O)N(C)c1cc2ccc1Nc1nc(ncc1Cl)Nc1cc(c(N3CCN(C)CC3)cc1OC)CCCCCO2] 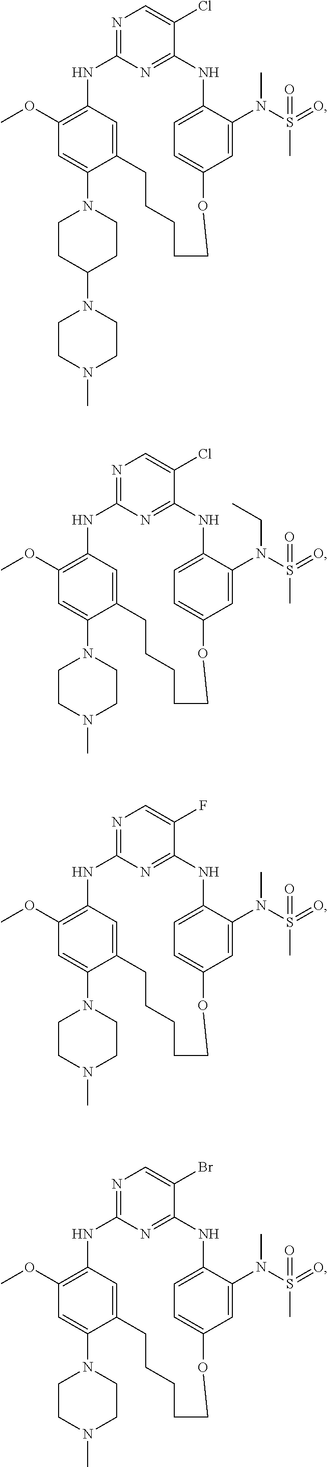 [SMILES: COc1cc(N2CCC(N3CCN(C)CC3)CC2)c2cc1Nc1ncc(Cl)c(n1)Nc1ccc(cc1N(C)S(C)(=O)=O)OCCCCC2 ; CCN(c1cc2ccc1Nc1nc(ncc1Cl)Nc1cc(c(N3CCN(C)CC3)cc1OC)CCCCCO2)S(C)(=O)=O ; COc1cc(N2CCN(C)CC2)c2cc1Nc1ncc(F)c(n1)Nc1ccc(cc1N(C)S(C)(=O)=O)OCCCCC2 ; COc1cc(N2CCN(C)CC2)c2cc1Nc1ncc(Br)c(n1)Nc1ccc(cc1N(C)S(C)(=O)=O)OCCCCC2] 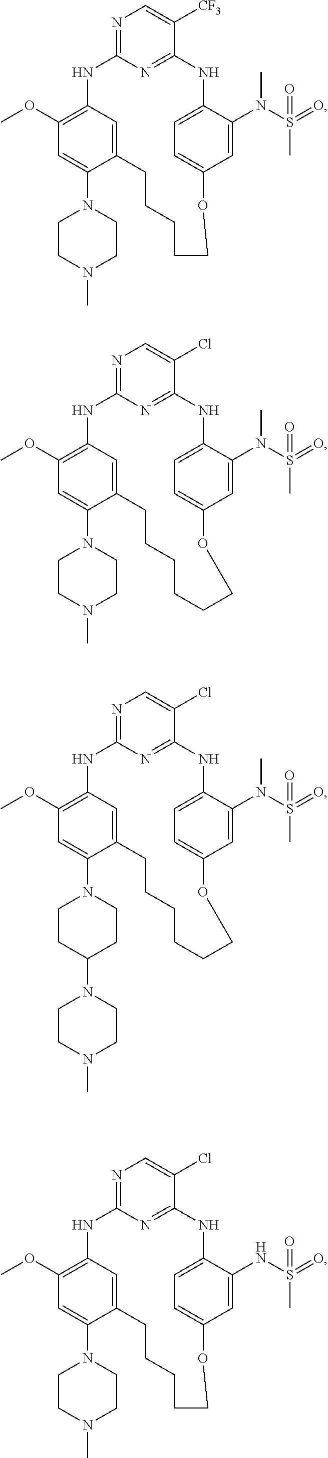 [SMILES: COc1cc(N2CCN(C)CC2)c2cc1Nc1ncc(C(F)(F)F)c(n1)Nc1ccc(cc1N(C)S(C)(=O)=O)OCCCCC2 ; COc1cc(N2CCN(C)CC2)c2cc1Nc1ncc(Cl)c(n1)Nc1ccc(cc1N(C)S(C)(=O)=O)OCCCCCC2 ; COc1cc(N2CCC(N3CCN(C)CC3)CC2)c2cc1Nc1ncc(Cl)c(n1)Nc1ccc(cc1N(C)S(C)(=O)=O)OCCCCCC2 ; COc1cc(N2CCN(C)CC2)c2cc1Nc1ncc(Cl)c(n1)Nc1ccc(cc1NS(C)(=O)=O)OCCCCC2] 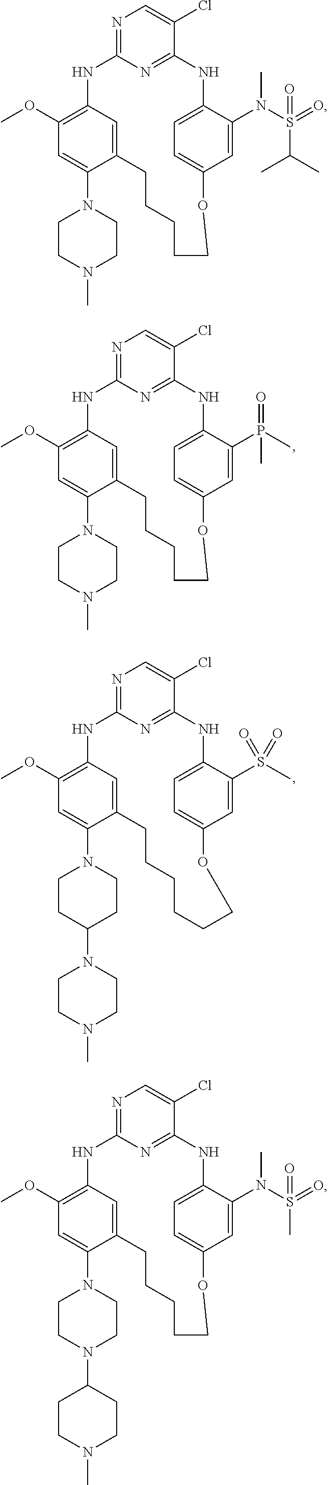 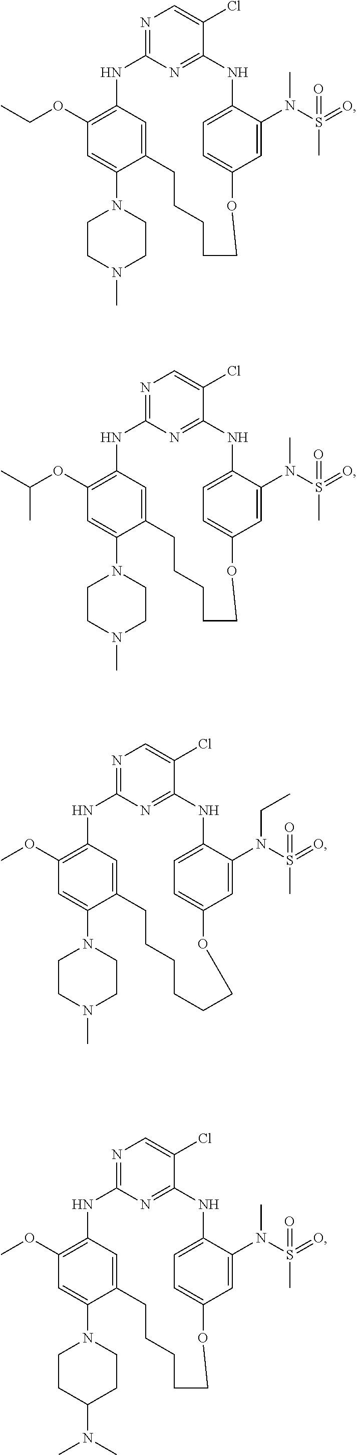 [SMILES: CCOc1cc(N2CCN(C)CC2)c2cc1Nc1ncc(Cl)c(n1)Nc1ccc(cc1N(C)S(C)(=O)=O)OCCCCC2 ; CC(C)Oc1cc(N2CCN(C)CC2)c2cc1Nc1ncc(Cl)c(n1)Nc1ccc(cc1N(C)S(C)(=O)=O)OCCCCC2 ; CCN(c1cc2ccc1Nc1nc(ncc1Cl)Nc1cc(c(N3CCN(C)CC3)cc1OC)CCCCCCO2)S(C)(=O)=O ; COc1cc(N2CCC(N(C)C)CC2)c2cc1Nc1ncc(Cl)c(n1)Nc1ccc(cc1N(C)S(C)(=O)=O)OCCCCC2] 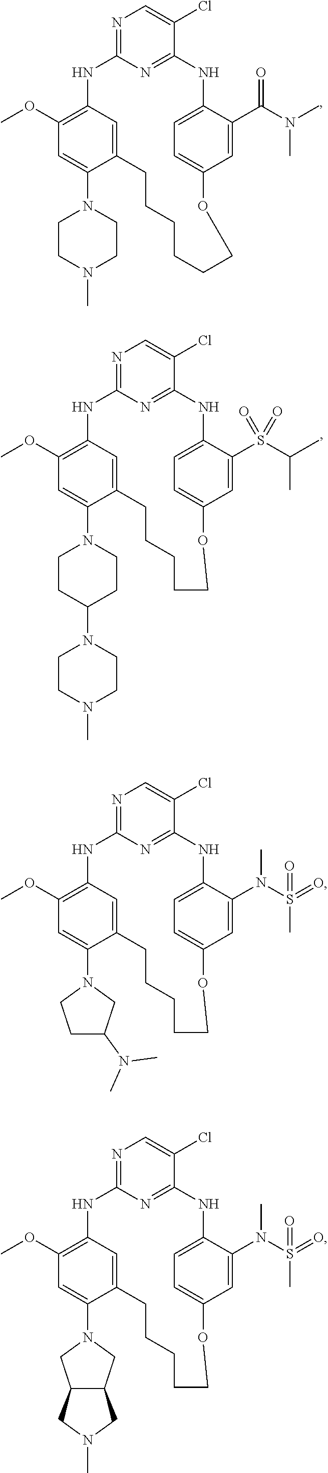 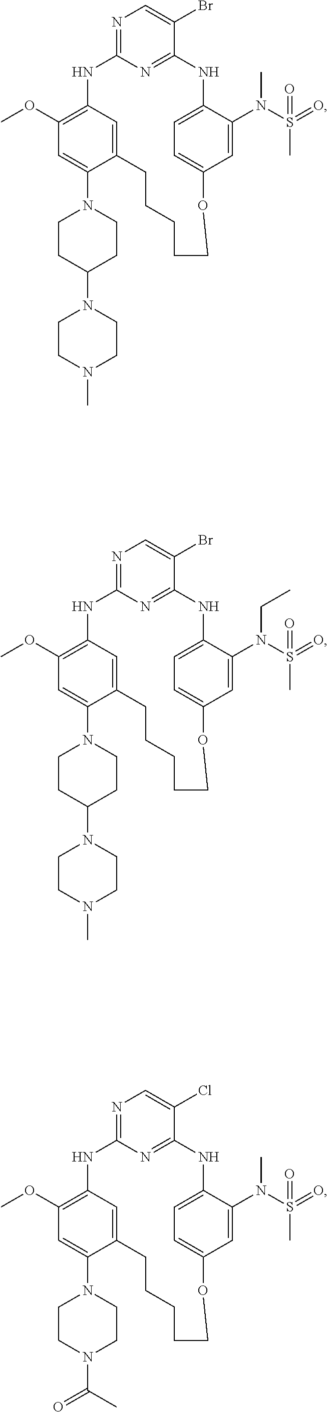 [SMILES: COc1cc(N2CCC(N3CCN(C)CC3)CC2)c2cc1Nc1ncc(Br)c(n1)Nc1ccc(cc1N(C)S(C)(=O)=O)OCCCCC2 ; CCN(c1cc2ccc1Nc1nc(ncc1Br)Nc1cc(c(N3CCC(N4CCN(C)CC4)CC3)cc1OC)CCCCCO2)S(C)(=O)=O ; COc1cc(N2CCN(C(C)=O)CC2)c2cc1Nc1ncc(Cl)c(n1)Nc1ccc(cc1N(C)S(C)(=O)=O)OCCCCC2] 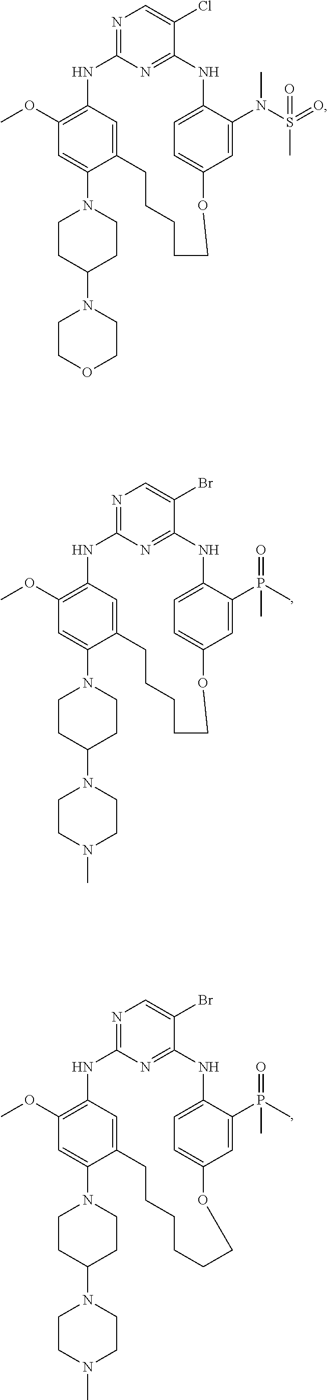 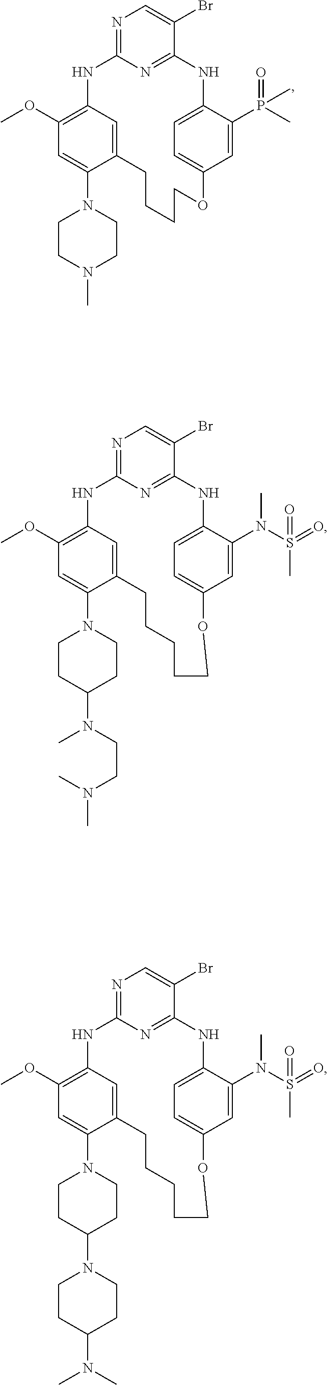 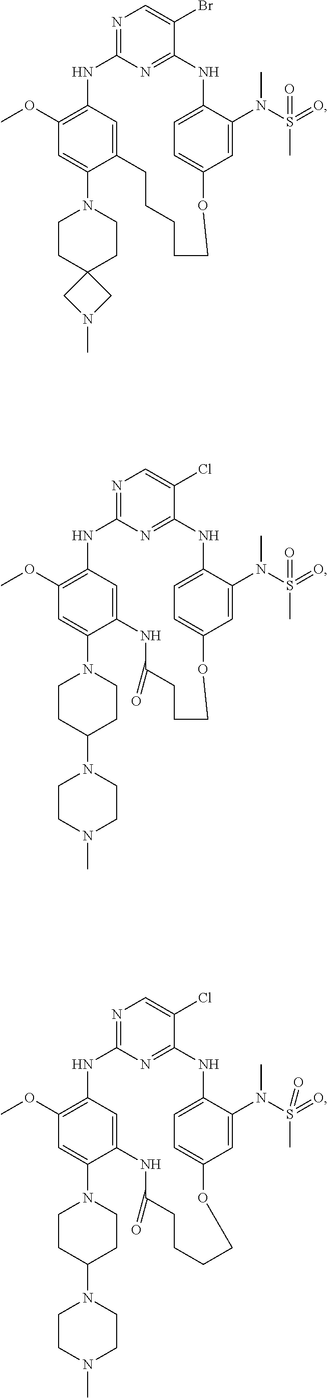 [SMILES: COc1cc(N2CCC3(CC2)CN(C)C3)c2cc1Nc1ncc(Br)c(n1)Nc1ccc(cc1N(C)S(C)(=O)=O)OCCCCC2 ; COc1cc(N2CCC(N3CCN(C)CC3)CC2)c2cc1Nc1ncc(Cl)c(n1)Nc1ccc(cc1N(C)S(C)(=O)=O)OCCCC(=O)N2 ; COc1cc(N2CCC(N3CCN(C)CC3)CC2)c2cc1Nc1ncc(Cl)c(n1)Nc1ccc(cc1N(C)S(C)(=O)=O)OCCCCC(=O)N2] 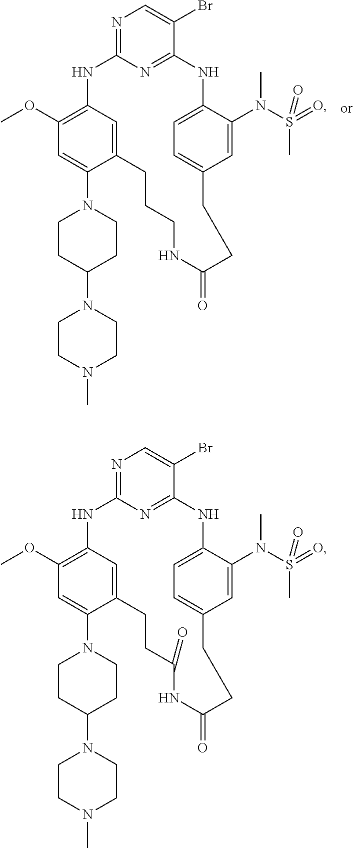 [SMILES: COc1cc(N2CCC(N3CCN(C)CC3)CC2)c2cc1Nc1ncc(Br)c(n1)Nc1ccc(cc1N(C)S(C)(=O)=O)CCC(=O)NCCC2 ; COc1cc(N2CCC(N3CCN(C)CC3)CC2)c2cc1Nc1ncc(Br)c(n1)Nc1ccc(cc1N(C)S(C)(=O)=O)CCC(=O)NC(=O)CC2]
wherein each structure is optionally substituted.

42. (canceled)

43. A pharmaceutical composition comprising a therapeutically effective amount of a compound of claim 1 to a mammal in need thereof and at least one pharmaceutically acceptable carrier.

44. A method of treating a disease, a condition, or a disorder which responds to the inhibition of epidermal growth factor receptor (EGFR), anaplastic lymphoma kinase (ALK) activity, or a combination thereof, comprising administering a compound of claim 1, or a pharmaceutically acceptable salt thereof to a mammal in need thereof.

45. (canceled)

46. (canceled)

47. The method of claim 44, wherein the disease is a cancer.
